(19) United States
(12) Patent Application Publication
(10) Pub. No.: US 2006/0269975 A1
(43) Pub. Date: Nov. 30, 2006

(54) Corynebacterium glutamicum genes encoding proteins involved in DNA replication, protein synthesis, and pathogenesis

(75) Inventors: Markus Pompejus, Freinsheim (DE); Burkhard Kroger, Limburgerhof (DE); Hartwig Schroder, Nussloch (DE); Oskar Zelder, Speyer (DE); Gregor Haberhauer, Limburgerhof (DE)

Correspondence Address: LAHIVE & COCKFIELD, 28 STATE STREET, BOSTON, MA, 02109, US

(73) Assignee: BASF Aktiengesellschaft, Ludwigshafen (DE)

(21) Appl. No.: 11/370,121

(22) Filed: Mar. 7, 2006

Related U.S. Application Data
(62) Division of application No. 09/604,693, filed on Jun. 27, 2000.
(60) Provisional application No. 60/144,448, filed on Jul. 16, 1999.
(60) Provisional application No. 60/149,402, filed on Aug. 17, 1999.

(51) Int. Cl. G01N 33/554; C07H 21/04; C12P 21/06; C12P 13/04; C12N 9/10; C12N 15/74; C12N 1/21

(52) U.S. Cl. 435/7.32; 536/23.2; 435/106; 435/69.1; 435/193; 435/252.3; 435/471

(57) ABSTRACT

Isolated nucleic acid molecules, designated RRP nucleic acid molecules, which encode novel RRP proteins from Corynebacterium glutamicum are described. The invention also provides antisense nucleic acid molecules, recombinant expression vectors containing RRP nucleic acid molecules, and host cells into which the expression vectors have been introduced. The invention still further provides isolated RRP proteins, mutated RRP proteins, fusion proteins, antigenic peptides and methods for the improvement of production of a desired compound from C. glutamicum based on genetic engineering of RRP genes in this organism.

This application is a divisional of U.S. application Ser. No. 09/604,693, filed Jun. 27, 2000 which claims priority to prior filed U.S. Provisional Patent Application Ser. No. 60/144448, filed Jul. 16, 1999, and U.S. Provisional Patent Application Ser. No. 60/149402, filed Aug. 17, 1999. The entire contents of all of the aforementioned applications are hereby expressly incorporated herein by this reference.

### INCORPORATION OF MATERIAL SUBMITTED ON COMPACT DISKS

This application incorporates herein by reference the material contained on the compact disks submitted herewith as part of this application. Specifically, the file “seqlistcorrtext” (1.60 MB) contained on each of Copy 1, Copy 2 and the CRF copy of the Sequence Listing is hereby incorporated herein by reference. This file was created on Mar. 7, 2006. In addition the files “Appendix A” (556 KB) and “Appendix B” (200 KB) contained on each of the compact disks entitled “Appendices Copy 1” and “Appendices Copy 2” are hereby incorporated herein by reference. Each of these files were created on Mar. 7, 2006.

### BACKGROUND OF THE INVENTION

Certain products and by-products of naturally-occurring metabolic processes in cells have utility in a wide array of industries, including the food, feed, cosmetics, and pharmaceutical industries. These molecules, collectively termed ‘fine chemicals’, include organic acids, both proteinogenic and non-proteinogenic amino acids, nucleotides and nucleosides, lipids and fatty acids, diols, carbohydrates, aromatic compounds, vitamins and cofactors, and enzymes. Their production is most conveniently performed through the large-scale culture of bacteria developed to produce and secrete large quantities of one or more desired molecules. One particularly useful organism for this purpose is Corynebacterium glutamicum, a gram positive bacterium lacking human pathogenicity. Through strain selection, a number of mutant strains have been developed which produce an array of desirable compounds. However, selection of strains improved for the production of a particular molecule is a time-consuming and difficult process.

### SUMMARY OF THE INVENTION

The invention provides novel bacterial nucleic acid molecules which have a variety of uses. These uses include the identification of microorganisms which can be used to produce fine chemicals, the modulation of fine chemical production in C. glutamicum or related bacteria, the typing or identification of C. glutamicum or related bacteria, as reference points for mapping the C. glutamicum genome, and as markers for transformation. These novel nucleic acid molecules encode proteins, referred to herein as DNA replication, ribosome and pathogenesis (RRP) proteins.

C. glutamicum is a gram positive, aerobic bacterium which is commonly used in industry for the large-scale production of a variety of fine chemicals, and also for the degradation of hydrocarbons (such as in petroleum spills) and for the oxidation of terpenolds. The RRP nucleic acid molecules of the invention, therefore, can be used to identify microorganisms which can be used to produce fine chemicals, e.g., by fermentation processes. Modulation of the expression of the RRP nucleic acids of the invention, or modification of the sequence of the RRP nucleic acid molecules of the invention, can be used to modulate the production of one or more fine chemicals from a microorganism (e.g., to improve the yield or production of one or more fine chemicals from a Corynebacterium or Brevibacterium species).

The RRP nucleic acids of the invention may also be used to identify an organism as being Corynebacterium glutamicum or a close relative thereof, or to identify the presence of C. glutamicum or a relative thereof in a mixed population of microorganisms. The invention provides the nucleic acid sequences of a number of C. glutamicum genes; by probing the extracted genomic DNA of a culture of a unique or mixed population of microorganisms under stringent conditions with a probe spanning a region of a C. glutamicum gene which is unique to this organism, one can ascertain whether this organism is present. Although Corynebacterium glutamicum itself is nonpathogenic, it is related to species pathogenic in humans, such as Corynebacterium diphtheriae (the causative agent of diphtheria); the detection of such organisms is of significant clinical relevance.

The RRP nucleic acid molecules of the invention may also serve as reference points for mapping of the C. glutamicum genome, or of genomes of related organisms. Similarly, these molecules, or variants or portions thereof, may serve as markers for genetically engineered Corynebacterium or Brevibacterium species.

The RRP proteins encoded by the novel nucleic acid molecules of the invention are capable of, for example, performing a function in C. glutamicum involved in the replication of DNA, in protein synthesis, or of contributing to the pathogenicity of the microorganism. Given the availability of cloning vectors for use in Corynebacterium glutamicum, such as those disclosed in Sinskey et al., U.S. Pat. No. 4,649,119, and techniques for genetic manipulation of C. glutamicum and the related Brevibacterium species (e.g., lactofermentum) (Yoshihama et al, J. Bacteriol. 162: 591-597 (1985); Katsumata et al., J. Bacteriol. 159: 306-311 (1984); and Santamaria et al., J. Gen. Microbiol. 130: 2237-2246 (1984)), the nucleic acid molecules of the invention may be utilized in the genetic engineering of this organism to make it a better or more efficient producer of one or more fine chemicals. This improved production or efficiency of production of a fine chemical may be due to a direct effect of manipulation of a gene of the invention, or it may be due to an indirect effect of such manipulation.

There are a number of mechanisms by which the alteration of an RRP protein of the invention may affect the yield, production, and/or efficiency of production of a fine chemical from a C. glutamicum strain incorporating such an altered protein. For example, by improving the rate at which DNA replication occurs (e.g. by optimizing the activity of one or more DNA polymerase, or by improving the rate at which the topoisomerases or helicases of the inventibn unwind DNA) it may be possible to increase the rate of cell division, which in turn increases the number of viable fine-chemical-producing C. glutamicum cells present in large-scale culture settings. Similarly, by improving the rate at which mRNA is translated to protein (e.g., by optimizing the activity of one or more of the ribosomal proteins) it may be possible to increase the number of proteins in the cell which participate in the synthesis of one or more desired fine chemicals, or in an overall increase in the rate of cell division (due to increased growth and metabolism), both of which should lead to increased production of one or more fine chemicals from large-scale fermentor cultures of these microorganisms. Alterations in the DNA replication proteins of the invention may also permit increased fidelity in the replicative process, thereby increasing the genetic stability and viability of the microorganism and lessening the chance that another engineered mutation improving fine chemical production from the microorganism will not be inadvertently mutagenized by error-prone replication. The RRP proteins of the invention involved in pathogenesis are themselves fine chemicals; by increasing the number or by engineering the corresponding genes such that the expression of these proteins is removed from cellular repression pathways, or by mutagenizing the proteins such that feedback regulatory regions are removed, it may be possible to increase the yield, production, and/or efficiency of production of these proteins from large-scale-fermentor culture of organisms containing such mutations.

The invention provides novel nucleic acid molecules which encode proteins, referred to herein as RRP proteins, which are capable of, for example, performing a function in C. glutamicum involved in the replication of DNA, in protein synthesis, or of contributing to the pathogenicity of the microorganism. Nucleic acid molecules encoding an RRP protein are referred to herein as RRP nucleic acid molecules. In a preferred embodiment, an RRP protein participates in C. glutamicum DNA replication, ribosome function, or in the pathogenesis of the organism, or possesses a C. glutamicum enzymatic or proteolytic activity. Examples of such proteins include those encoded by the genes set forth in Table 1.

Accordingly, one aspect of the invention pertains to isolated nucleic acid molecules (e.g., cDNAs, DNAs, or RNAs) comprising a nucleotide sequence encoding an RRP protein or biologically active portions thereof, as well as nucleic acid fragments suitable as primers or hybridization probes for the detection or amplification of RRP-encoding nucleic acids (e.g., DNA or mRNA). In particularly preferred embodiments, the isolated nucleic acid molecule comprises one of the nucleotide sequences set forth in Appendix A or the coding region or a complement thereof of one of these nucleotide sequences. In other particularly preferred embodiments, the isolated nucleic acid molecule of the invention comprises a nucleotide sequence which hybridizes to or is at least about 50%, preferably at least about 60%, more preferably at least about 70%, 80% or 90%, and even more preferably at least about 95%, 96%, 97%, 98%, 99% or more homologous to a nucleotide sequence set forth in Appendix A, or a portion thereof. In other preferred embodiments; the isolated nucleic acid molecule encodes one of the amino acid sequences set forth in Appendix B. The preferred RRP proteins of the present invention also preferably possess at least one of the RRP activities described herein.

In another embodiment, the isolated nucleic acid molecule encodes a protein or portion thereof wherein the protein or portion thereof includes an amino acid sequence which is sufficiently homologous to an amino acid sequence of Appendix B, e.g., sufficiently homologous to an amino acid sequence of Appendix B such that the protein or portion thereof maintains an RRP activity. Preferably, the protein or portion thereof encoded by the nucleic acid molecule maintains the ability to participate in the replication of DNA, in protein synthesis, or in the pathogenicity of the microorganism. In one embodiment, the protein encoded by the nucleic acid molecule is at least about 50%, preferably at least about 60%, and more preferably at least about 70%, 80%, or 90% and most preferably at least about 95%, 96%, 97%, 98%, or 99% or more homologous to an amino acid sequence of Appendix B (e.g., an entire amino acid sequence selected from those sequences set forth in Appendix B). In another preferred embodiment, the protein is a full length C. glutamicum protein which is substantially homologous to an entire amino acid sequence of Appendix B (encoded by an open reading frame shown in Appendix A).

In another preferred embodiment, the isolated nucleic acid molecule is derived from C. glutamicum and encodes a protein (e.g., an RRP fusion protein) which includes a biologically active domain which is at least about 50% or more homologous to one of the amino acid sequences of Appendix B and is able to participate in the replication of C. glutamicum DNA, in C. glutamicum protein synthesis, or may contribute to the pathogenicity of the microorganism, or has one or more of the activities set forth in Table 1, and which also includes heterologous nucleic acid sequences encoding a heterologous polypeptide or regulatory regions.

In another embodiment, the isolated nucleic acid molecule is at least 15 nucleotides in length and hybridizes under stringent conditions to a nucleic acid molecule comprising a nucleotide sequence of Appendix A. Preferably, the isolated nucleic acid molecule corresponds to a naturally-occurring nucleic acid molecule. More preferably, the isolated nucleic acid encodes a naturally-occurring C. glutamicum RRP protein, or a biologically active portion thereof.

Another aspect of the invention pertains to vectors, e.g., recombinant expression vectors, containing the nucleic acid molecules of the invention, and host cells into which such vectors have been introduced. In one embodiment, such a host cell is used to produce an RRP protein by culturing the host cell in a suitable medium. The RRP protein can be then isolated from the medium or the host cell.

Yet another aspect of the invention pertains to a genetically altered microorganism in which an RRP gene has been introduced or altered. In one embodiment, the genome of the microorganism has been altered by introduction of a nucleic acid molecule of the invention encoding wild-type or mutated RRP sequence as a transgene. In another embodiment, an endogenous RRP gene within the genome of the microorganism has been altered, e.g., functionally disrupted, by homologous recombination with an altered RRP gene. In another embodiment, an endogenous or introduced RRP gene in a microorganism has been altered by one or more point mutations, deletions, or inversions, but still encodes a functional RRP protein. In still another embodiment, one or more of the regulatory regions (e.g., a promoter, repressor, or inducer) of an RRP gene in a microorganism has been altered (e.g., by deletion, truncation, inversion, or point mutation) such that the expression of the RRP gene is modulated. In a preferred embodiment, the microorganism belongs to the genus Corynebacterium or Brevibacterium, with Corynebacterium glutamicum being particularly preferred. In a preferred embodiment, the microorganism is also utilized for the production of a desired compound, such as an amino acid, with lysine being particularly preferred.

In another aspect, the invention provides a method of identifying the presence or activity of Cornyebacterium diphtheriae in a subject. This method includes detection of one or more of the nucleic acid or amino acid sequences of the invention (e.g., the sequences set forth in Appendix A or Appendix B) in a subject, thereby detecting the presence or activity of Corynebacterium diphtheriae in the subject.

Still another aspect of the invention pertains to an isolated RRP protein or a portion, e.g., a biologically active portion, thereof. In a preferred embodiment, the isolated RRP protein or portion thereof can participate in the replication of C. glutamicum DNA, in C. glutamicum protein synthesis, or may contribute to the pathogenicity of the microorganism. In another preferred embodiment, the isolated RRP protein or portion thereof is sufficiently homologous to an amino acid sequence of Appendix B such that the protein or portion thereof maintains the ability to participate in the replication of C. glutamicum DNA, to participate in C. glutamicum protein synthesis, or may contribute to the pathogenicity of the microorganism.

The invention also provides an isolated preparation of an RRP protein. In preferred embodiments, the RRP protein comprises an amino acid sequence of Appendix B. In another preferred embodiment, the invention pertains to an isolated full length protein which is substantially homologous to an entire amino acid sequence of Appendix B (encoded by an open reading frame set forth in Appendix A). In yet another embodiment, the protein is at least about 50%, preferably at least about 60%, and more preferably at least about 70%, 80%, or 90%, and most preferably at least about 95%, 96%, 97%, 98%, or 99% or more homologous to an entire amino acid sequence of Appendix B. In other embodiments, the isolated RRP protein comprises an amino acid sequence which is at least about 50% or more homologous to one of the amino acid sequences of Appendix B and is able to participate in the replication of C. glutamicum DNA, to participate in C. glutamicum protein synthesis, or may contribute to the pathogenicity of the microorganism, or has one or more of the activities set forth in Table 1.

Alternatively, the isolated RRP protein can comprise an amino acid sequence which is encoded by a nucleotide sequence which hybridizes, e.g., hybridizes under stringent conditions, or is at least about 50%, preferably at least about 60%, more preferably at least about 70%, 80%, or 90%, and even more preferably at least about 95%, 96%, 97%, 98,%, or 99% or more homologous, to a nucleotide sequence of Appendix B. It is also preferred that the preferred forms of RRP proteins also have one or more of the RRP bioactivities described herein.

The RRP polypeptide, or a biologically active portion thereof, can be operatively linked to a non-RRP polypeptide to form a fusion protein. In preferred embodiments, this fusion protein has an activity which differs from that of the RRP protein alone. In other preferred embodiments, this fusion protein participates in the replication of C. glutamicum DNA, participates in C. glutamicum protein synthesis, or contributes to the pathogenicity of the microorganism. In particularly preferred embodiments, integration of this fusion protein into a host cell modulates production of a desired compound from the cell.

In another aspect, the invention provides methods for screening molecules which modulate the activity of an RRP protein, either by interacting with the protein itself or a substrate or binding partner of the RRP protein, or by modulating the transcription or translation of an RRP nucleic acid molecule of the invention.

Another aspect of the invention pertains to a method for producing a fine chemical. This method involves the culturing of a cell containing a vector directing the expression of an RRP nucleic acid molecule of the invention, such that a fine chemical is produced. In a preferred embodiment, this method further includes the step of obtaining a cell containing such a vector, in which a cell is transfected with a vector directing the expression of an RRP nucleic acid. In another preferred embodiment, this method further includes the step of recovering the fine chemical from the culture. In a particularly preferred embodiment, the cell is from the genus Corynebacterium or Brevibacterium, or is selected from those strains set forth in Table 3.

Another aspect of the invention pertains to methods for modulating production of a molecule from a microorganism. Such methods include contacting the cell with an agent which modulates RRP protein activity or RRP nucleic acid expression such that a cell associated activity is altered relative to this same activity in the absence of the agent. In a preferred embodiment, the cell is modulated for one or more C. glutamicum processes involved in the replication of C. glutamicum DNA, in C. glutamicum protein synthesis, or in the pathogenicity of the microorganism. The agent which modulates RRP protein activity can be an agent which stimulates RRP protein activity or RRP nucleic acid expression. Examples of agents which stimulate RRP protein activity or RRP nucleic acid expression include small molecules, active RRP proteins, and nucleic acids encoding RRP proteins that have been introduced into the cell. Examples of agents which inhibit RRP activity or expression include small molecules and antisense RRP nucleic acid molecules.

Another aspect of the invention pertains to methods for modulating yields of a desired compound from a cell, involving the introduction of a wild-type or mutant RRP gene into a cell, either maintained on a separate plasmid or integrated into the genome of the host cell. If integrated into the genome, such integration can be random, or it can take place by homologous recombination such that the native gene is replaced by the introduced copy, causing the production of the desired compound from the cell to be modulated. In a preferred embodiment, said yields are increased. In another preferred embodiment, said chemical is a fine chemical. In a particularly preferred embodiment, said fine chemical is an amino acid. In especially preferred embodiments, said amino acid is L-lysine.

### DETAILED DESCRIPTION OF THE INVENTION

The present invention provides RRP nucleic acid and protein molecules which are involved in C. glutamicum DNA replication, protein synthesis, or pathogenesis. The molecules of the invention may be utilized in the modulation of production of fine chemicals from microorganisms, such as C. glutamicum, either directly (e.g., where increased activity of a ribosome permits increased production of fine chemical biosynthetic proteins, which may result in increased yields, production, or efficiency of production of one or more fine chemicals from the modified C. glutamicum), or may have an indirect impact which nonetheless results in an increase of yield, production, and/or efficiency of production of the desired compound (e.g., where modulation of the activity or number of copies of a C. glutamicum DNA synthesis protein results in an increase in the rate of C. glutamicum cell division, resulting in greater numbers of viable cells in culture, which in turn permits increased production in a large-scale culture setting). Aspects of the invention are further explicated below.

I. Fine Chemicals

The term ‘fine chemical’ is art-recognized and includes molecules produced by an organism which have applications in various industries, such as, but not limited to, the pharmaceutical, agriculture, and cosmetics industries. Such compounds include organic acids, such as tartaric acid, itaconic acid, and diaminopimelic acid, both proteinogenic and non-proteinogenic amino acids, purine and pyrimidine bases, nucleosides, and nucleotides (as described e.g. in Kuninaka, A. (1996) Nucleotides and related compounds, p. 561-612, in Biotechnology vol. 6, Rehm et al., eds. VCH: Weinheim, and references contained therein), lipids, both saturated and unsaturated fatty acids (e.g., arachidonic acid), diols (e.g., propane diol, and butane diol), carbohydrates (e.g., hyaluronic acid and trehalose), aromatic compounds (e.g., aromatic amines, vanillin, and indigo), vitamins and cofactors (as described in Ullmann's Encyclopedia of Industrial Chemistry, vol. A27, “Vitamins”, p. 443-613 (1996) VCH: Weinheim and references therein; and Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sep. 1-3, 1994 at Penang, Malaysia, AOCS Press, (1995)), enzymes, polyketides (Cane et al. (1998) Science 282: 63-68), toxins, and all other chemicals described in Gutcho (1983) Chemicals by Fermentation, Noyes Data Corporation, ISBN: 0818805086 and references therein. The metabolism and uses of certain of these fine chemicals are further explicated below.

A. Amino Acid Metabolism and Uses

Amino acids comprise the basic structural units of all proteins, and as such are essential for normal cellular functioning in all organisms. The term “amino acid” is art-recognized. The proteinogenic amino acids, of which there are 20 species, serve as structural units for proteins, in which they are linked by peptide bonds, while the nonproteinogenic amino acids (hundreds of which are known) are not normally found in proteins (see Ulmann's Encyclopedia of Industrial Chemistry, vol. A2, p. 57-97 VCH: Weinheim (1985)). Amino acids may be in the D- or L-optical configuration, though L-amino acids are generally the only type found in naturally-occurring proteins. Biosynthetic and degradative pathways of each of the 20 proteinogenic amino acids have been well characterized in both prokaryotic and eukaryotic cells (see, for example, Stryer, L. Biochemistry, 3^{rd} edition, pages 578-590 (1988)). The ‘essential’ amino acids (histidine, isoleucine, leucine, lysine, methionine, phenylalanine, threonine, tryptophan, and valine), so named because they are generally a nutritional requirement due to the complexity of their biosyntheses, are readily converted by simple biosynthetic pathways to the remaining 11 ‘nonessential’ amino acids (alanine, arginine, asparagine, aspartate, cysteine, glutamate, glutamine, glycine, proline, serine, and tyrosine). Higher animals do retain the ability to synthesize some of these amino acids, but the essential amino acids must be supplied from the diet in order for normal protein synthesis to occur.

Aside from their function in protein biosynthesis, these amino acids are interesting chemicals in their own right; and many have been found to have various applications in the food, feed, chemical, cosmetics, agriculture, and pharmaceutical industries. Lysine is an important amino acid in the nutrition not only of humans, but also of monogastric animals such as poultry and swine. Glutamate is most commonly used as a flavor additive (mono-sodium glutamate, MSG) and is widely used throughout the food industry, as are aspartate, phenylalanine, glycine, and cysteine. Glycine, L-methionine and tryptophan are all utilized in the pharmaceutical industry. Glutamine, valine, leucine, isoleucine, histidine, arginine, proline, serine and alanine are of use in both the pharmaceutical and cosmetics industries. Threonine, tryptophan, and D/L-methionine are common feed additives. (Leuchtenberger, W. (1996) Amino aids—technical production and use, p. 466-502 in Rehm et al. (eds.) Biotechnology vol. 6, chapter 14a, VCH: Weinheim). Additionally, these amino acids have been found to be useful as precursors for the synthesis of synthetic amino acids and proteins, such as N-acetylcysteine, S-carboxymethyl-L-cysteine, (S)-5-hydroxytryptophan, and others described in Ulmann's Encyclopedia of Industrial Chemistry, vol. A2, p. 57-97, VCH: Weinheim, 1985.

The biosynthesis of these natural amino acids in organisms capable of producing them, such as bacteria, has been well characterized (for review of bacterial amino acid biosynthesis and regulation thereof, see Umbarger, H. E. (1978) Ann. Rev. Biochem. 47: 533-606). Glutamate is synthesized by the reductive amination of α-ketoglutarate, an intermediate in the citric acid cycle. Glutamine, proline, and arginine are each subsequently produced from glutamate. The biosynthesis of serine is a three-step process beginning with 3-phosphoglycerate (an intermediate in glycolysis), and resulting in this amino acid after oxidation, transamination, and hydrolysis steps. Both cysteine and glycine are produced from serine; the former by the condensation of homocysteine with serine, and the latter by the transferal of the side-chain β-carbon atom to tetrahydrofolate, in a reaction catalyzed by serine transhydroxymethylase. Phenylalanine, and tyrosine are synthesized from the glycolytic and pentose phosphate pathway precursors erythrose 4-phosphate and phosphoenolpyruvate in a 9-step biosynthetic pathway that differ only at the final two steps after synthesis of prephenate. Tryptophan is also produced from these two initial molecules, but its synthesis is an 11-step pathway. Tyrosine may also be synthesized from phenylalanine, in a reaction catalyzed by phenylalanine hydroxylase. Alanine, valine, and leucine are all biosynthetic products of pyruvate, the final product of glycolysis. Aspartate is formed from oxaloacetate, an intermediate of the citric acid cycle. Asparagine, methionine, threonine, and lysine are each produced by the conversion of aspartate. Isoleucine is formed from threonine. A complex 9-step pathway results in the production of histidine from 5-phosphoribosyl-1-pyrophosphate, an activated sugar.

Amino acids in excess of the protein synthesis needs of the cell cannot be stored, and are instead degraded to provide intermediates for the major metabolic pathways of the cell (for review see Stryer, L. Biochemistry 3^{rd} ed. Ch. 21 “Amino Acid Degradation and the Urea Cycle” p. 495-516 (1988)). Although the cell is able to convert unwanted amino acids into useful metabolic intermediates, amino acid production is costly in terms of energy, precursor molecules, and the enzymes necessary to synthesize them. Thus it is not surprising that amino acid biosynthesis is regulated by feedback inhibition, in which the presence of a particular amino acid serves to slow or entirely stop its own production (for overview of feedback mechanisms in amino acid biosynthetic pathways, see Stryer, L. Biochemistry, 3^{rd} ed. Ch. 24: “Biosynthesis of Amino Acids and Heme” p. 575-600 (1988)). Thus, the output of any particular amino acid is limited by the amount of that amino acid present in the cell.

B. Vitamin, Cofactor, and Nutraceutical Metabolism and Uses

Vitamins, cofactors, and nutraceuticals comprise another group of molecules which the higher animals have lost the ability to synthesize and so must ingest, although they are readily synthesized by other organisms such as bacteria. These molecules are either bioactive substances themselves, or are precursors of biologically active substances which may serve as electron carriers or intermediates in a variety of metabolic pathways. Aside from their nutritive value, these compounds also have significant industrial value as coloring agents, antioxidants, and catalysts or other processing aids. (For an overview of the structure, activity, and industrial applications of these compounds, see, for example, Ullman's Encyclopedia of Industrial Chemistry, “Vitamins” vol. A27, p. 443-613, VCH: Weinheim, 1996.) The term “vitamin” is art-recognized, and includes nutrients which are required by an organism for normal functioning, but which that organism cannot synthesize by itself. The group of vitamins may encompass cofactors and nutraceutical compounds. The language “cofactor” includes nonproteinaceous compounds required for a normal enzymatic activity to occur. Such compounds may be organic or inorganic; the cofactor molecules of the invention are preferably organic. The term “nutraceutical” includes dietary supplements having health benefits in plants and animals, particularly humans. Examples of such molecules are vitamins, antioxidants, and also certain lipids (e.g., polyunsaturated fatty acids).

The biosynthesis of these molecules in organisms capable of producing them, such as bacteria, has been largely characterized (Ullman's Encyclopedia of Industrial Chemistry, “Vitamins” vol. A27,p. 443-613; VCH: Weinheim, 1996; Michal, G. (1999) Biochemical Pathways: An Atlas of Biochemistry and Molecular Biology, John Wiley & Sons; Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sep. 1-3, 1994 at Penang, Malaysia, AOCS Press: Champaign, Ill. X, 374 S).

Thiamin (vitamin B₁) is produced by the chemical coupling of pyrimidine and thiazole moieties. Riboflavin (vitamin B₂) is synthesized from guanosine-5′-triphosphate (GTP) and ribose-5′-phosphate. Riboflavin, in turn, is utilized for the synthesis of flavin mononucleotide (FMN) and flavin adenine dinucleotide (FAD). The family of compounds collectively termed ‘vitamin B₆’ (e.g., pyridoxine, pyridoxamine, pyridoxa-5′-phosphate, and the commercially used pyridoxin hydrochloride) are all derivatives of the common structural unit, 5-hydroxy-6-methylpyridine. Pantothenate (pantothenic acid, (R)-(+)-N-(2,4-dihydroxy-3,3-dimethyl-1-oxobutyl)-β-alanine) can be produced either by chemical synthesis or by fermentation. The final steps in pantothenate biosynthesis consist of the ATP-driven condensation of β-alanine and pantoic acid. The enzymes responsible for the biosynthesis steps for the conversion to pantoic acid, to β-alanine and for the condensation to panthotenic acid are known. The metabolically active form of pantothenate is Coenzyme A, for which the biosynthesis proceeds in 5 enzymatic steps. Pantothenate, pyridoxal-5′-phosphate, cysteine and ATP are the precursors of Coenzyme A. These enzymes not only catalyze the formation of panthothante, but also the production of (R)-pantoic acid, (R)-pantolacton, (R)-panthenol (provitamin B₅), pantetheine (and its derivatives) and coenzyme A.

Biotin biosynthesis from the precursor molecule pimeloyl-CoA in microorganisms has been studied in detail and several of the genes involved have been identified. Many of the corresponding proteins have been found to also be involved in Fe-cluster synthesis and are members of the nifS class of proteins. Lipoic acid is derived from octanoic acid, and serves as a coenzyme in energy metabolism, where it becomes part of the pyruvate dehydrogenase complex and the α-ketoglutarate dehydrogenase complex. The folates are a group of substances which are all derivatives of folic acid, which is turn is derived from L-glutamic acid, p-amino-benzoic acid and 6-methylpterin. The biosynthesis of folic acid and its derivatives, starting from the metabolism intermediates guanosine-5′-triphosphate (GTP), L-glutamic acid and p-amino-benzoic acid has been studied in detail in certain microorganisms.

Corrinoids (such as the cobalamines and particularly vitamin B₁₂) and porphyrines belong to a group of chemicals characterized by a tetrapyrole ring system. The biosynthesis of vitamin B₁₂ is sufficiently complex that it has not yet been completely characterized, but many of the enzymes and substrates involved are now known. Nicotinic acid (nicotinate), and nicotinamide are pyridine derivatives which are also termed ‘niacin’. Niacin is the precursor of the important coenzymes NAD (nicotinamide adenine dinucleotide) and NADP (nicotinamide adenine dinucleotide phosphate) and their reduced forms.

The large-scale production of these compounds has largely relied on cell-free chemical syntheses, though some of these chemicals have also been produced by large-scale culture of microorganisms, such as riboflavin, Vitamin B₆, pantothenate, and biotin. Only Vitamin B₁₂ is produced solely by fermentation, due to the complexity of its synthesis. In vitro methodologies require significant inputs of materials and time, often at great cost.

C. Purine, Pyrimidine, Nucleoside and Nucleotide Metabolism and Uses

Purine and pyrimidine metabolism genes and their corresponding proteins are important targets for the therapy of tumor diseases and viral infections. The language “purine” or “pyrimidine” includes the nitrogenous bases which are constituents of nucleic acids, co-enzymes, and nucleotides. The term “nucleotide” includes the basic structural units of nucleic acid molecules, which are comprised of a nitrogenous base, a pentose sugar (in the case of RNA, the sugar is ribose; in the case of DNA, the sugar is D-deoxyribose), and phosphoric acid. The language “nucleoside” includes molecules which serve as precursors to nucleotides, but which are lacking the phosphoric acid moiety that nucleotides possess. By inhibiting the biosynthesis of these molecules, or their mobilization to form nucleic acid molecules, it is possible to inhibit RNA and DNA synthesis; by inhibiting this activity in a fashion targeted to cancerous cells, the ability of tumor cells to divide and replicate may be inhibited. Additionally, there are nucleotides which do not form nucleic acid molecules, but rather serve as energy stores (i.e., AMP) or as coenzymes (i.e., FAD and NAD).

Several publications have described the use of these chemicals for these medical indications, by influencing purine and/or pyrimidine metabolism (e.g. Christopherson, R. I. and Lyons, S. D. (1990) “Potent inhibitors of de novo pyrimidine and purine biosynthesis as chemotherapeutic agents.” Med. Res. Reviews 10: 505-548). Studies of enzymes involved in purine and pyrimidine metabolism have been focused on the development of new drugs which can be used, for example, as immunosuppressants or anti-proliferants (Smith, J. L., (1995) “Enzymes in nucleotide synthesis.” Curr. Opin. Struct. Biol. 5: 752-757; (1995) Biochem. Soc. Transact. 23: 877-902). However, purine and pyrimidine bases, nucleosides and nucleotides have other utilities: as intermediates in the biosynthesis of several fine chemicals (e.g., thiamine, S-adenosyl-methionine, folates, or riboflavin), as energy carriers for the cell (e.g., ATP or GTP), and for chemicals themselves, commonly used as flavor enhancers (e.g., IMP or GMP) or for several medicinal applications (see, for example, Kuninaka, A. (1996) Nucleotides and Related Compounds in Biotechnology vol. 6, Rehm et al., eds. VCH: Weinheim, p. 561-612). Also, enzymes involved in purine, pyrimidine, nucleoside, or nucleotide metabolism are increasingly serving as targets against which chemicals for crop protection, including fungicides, herbicides and insecticides, are developed.

The metabolism of these compounds in bacteria has been characterized (for reviews see, for example, Zalkin, H. and Dixon, J. E. (1992) “de novo purine nucleotide biosynthesis”, in: Progress in Nucleic Acid Research and Molecular Biology, vol. 42, Academic Press:, p. 259-287; and Michal, G. (1999) “Nucleotides and Nucleosides”, Chapter 8 in: Biochemical Pathways: An Atlas of Biochemistry and Molecular Biology, Wiley: N.Y.). Purine metabolism has been the subject of intensive research, and is essential to the normal functioning of the cell. Impaired purine metabolism in higher animals can cause severe disease, such as gout. Purine nucleotides are synthesized from ribose-5-phosphate, in a series of steps through the intermediate compound inosine-5′-phosphate (IMP), resulting in the production of guanosine-5′-monophosphate (GMP) or adenosine-5′-monophosphate (AMP), from which the triphosphate forms utilized as nucleotides are readily formed. These compounds are also utilized as energy stores, so their degradation provides energy for many different biochemical processes in the cell. Pyrimidine biosynthesis proceeds by the formation of uridine-5′-monophosphate (UMP) from ribose-5-phosphate. UMP, in turn, is converted to cytidine-5′-triphosphate (CTP). The deoxy-forms of all of these nucleotides are produced in a one step reduction reaction from the diphosphate ribose form of the nucleotide to the diphosphate deoxyribose form of the nucleotide. Upon phosphorylation, these molecules are able to participate in DNA synthesis.

D. Trehalose Metabolism and Uses

Trehalose consists of two glucose molecules, bound in α,α-1,1 linkage. It is commonly used in the food industry as a sweetener, an additive for dried or frozen foods, and in beverages. However, it also has applications in the pharmaceutical, cosmetics and biotechnology industries (see, for example, Nishimoto et al., (1998) U.S. Pat. No. 5,759,610; Singer, M. A. and Lindquist, S. (1998) Trends Biotech. 16: 460-467; Paiva, C. L. A. and Panek, A. D. (1996) Biotech. Ann. Rev. 2: 293-314; and Shiosaka, M. (1997) J. Japan 172: 97-102). Trehalose is produced by enzymes from many microorganisms and is naturally released into the surrounding medium, from which it can be collected using methods known in the art.

II. Activities of the Genes of the Invention

In order for a population of a particular type of bacterial cells to survive in an environment, at least three activities are necessary. First, the cells must be able to divide efficiently, such that the population is at least maintained, if not increased. Second, the cells must be able to efficiently express those genes encoding proteins necessary for normal cellular functioning. Finally, the cells must be able to influence their interaction with the surrounding environment, either by adaptation to the prevailing environmental conditions, by physical movement to preferred surroundings, or by directly altering the surrounding environment such that their own viability is improved. Critical processes involved in each of the aforementioned activities include replication of the bacterial genome, the action of the ribosome in protein synthesis, and anticellular or cell lytic activities (such as those involved in the pathogenesis of an organism).

A. DNA Replication

In order for a cell (e.g., a bacterial cell) to divide to form viable progeny cells, the cellular genome must be replicated. This is a multistep process, in which the tightly packaged DNA must first be locally freed from topological constraints, the two strands of the double helix must be unwound, a DNA polymerase must synthesize a new strand of DNA complementary to one of the original strands, and both the old and the new strands must be repackaged. Each of these steps is described in greater detail in the following section (see, e.g., Michal, G. (1999) Biochemical Pathways: An Atlas of Biochemistry and Molecular Biology, Wiley: N.Y., and references therein; and Griffiths, A. J. F. et al., (1993) An Introduction to Genetic Analysis, 5^{th} ed., Freeman: N.Y. p. 304-332 and references therein).

The general structure of genomic DNA in bacterial cells has been characterized. Bacterial chromosomes are usually circular in nature, and bacterial cells may also contain one or more different types of plasmids (also circular pieces of DNA, although usually significantly smaller in size than the bacterial chromosome) which may be replicated and incorporated into the daughter cell simultaneously with the chromosome. Replication of either of these circular pieces of genetic information typically begins at a single designated origin of replication (ori). Replication of the DNA may then take place either in one direction around the circle (rolling circle replication) until the origin is again reached, or it may occur in both directions simultaneously (θ-mechanism).

The ori site has a particular structure which permits the initiation of replication. First, the ori region typically contains multiple sequences which serve as binding sites for initiator proteins. The binding of the initiator proteins. (e.g.,.DnaA in E. coli) to these binding sites at the origin takes place in an ATP-dependent fashion. Upon ATP hydrolysis, the DNA bends around these DNA-associated molecules, and the two strands of DNA at the site separate, forming an open complex.

The molecule responsible for the actual synthesis of the new DNA molecule is a DNA polymerase. For replication purposes, the DNA polymerase utilized by the cell is the DNA polymerase III (Pol III) holoenzyme. This complex comprises 10 molecules, each of which has a different function in the complex. For example, the dimeric γ subunit acts to associate the i subunit with a primed DNA template in an ATP-dependent fashion. The β subunit is the ‘processivity factor’—the portion of the holoenzyme which specifically associates with the DNA template and which permits the template to ‘slide’ along the DNA due to its ring-like structure. The α subunit catalyzes the reaction which adds the new dNTP to the nascent nucleotide strand, and the ε subunit contains the 3′-5′ exonuclease activity.

A significant topological barrier to DNA synthesis exists due to the structure of a DNA molecule and to that of the bacterial chromosome. Not only must the double helix of the DNA molecule be split such that a single strand may be replicated, but this unwinding process results in increased positive supercoiling of the chromosome. Two types of enzymes permit these processes to occur despite the topological constraints: helicase unwinds the double helix in an ATP-dependent fashion, introducing positive supercoils into the bacterial chromosome. Gyrase introduces negative supercoils into the bacterial genome (in an ATP-dependent fashion), counteracting the positive supercoils introduced by the helicase. The result of their combined is a replication fork: a split between the two strands of DNA in which replication of each strand of the DNA can occur. Single-stranded binding proteins (SSBS) bind to the unwound DNA molecules to prevent them from reassociating.

In order for Pol III to initiate DNA synthesis, it must have a sequence from which to prime synthesis. Primase (E. coli DnaG) synthesizes RNA primers as starting sequences for Pol III. The Pol III complex gamma subunit associates with the newly synthesized primers and subsequently associates with the dimeric beta Pol III subunits, initiating DNA synthesis. Replication of each strand takes place simultaneously, but because Pol III polymerizes dNTPs only in the 5′-3′ direction, only one strand (the 3′-5′ leading strand) can be continuously replicated. The other strand (the complementary lagging strand) is replicated in short fragments (Okazaki fragments), due to the lack of progressivity of the polymerase in this direction. These fragments are subsequently ligated by DNA ligase to form a single strand. Incorrectly added bases are excised by the 3′-5 exonuclease activity of Pol III and the nick sealed by DNA ligase.

Bacterial DNA replication is terminated at a site opposite to the origin at which terminator proteins bind. The association of these proteins with the DNA prevents the replication fork from progressing. The RNA primer used to initiate DNA synthesis is degraded by DNA polymerase I (Pol I) or ribonuclease H (RnaseH), and Pol I adds the appropriate dNTPs to the gap. Finally, DNA ligase seals the nicks. To achieve semiconservative replication, the two strands of the parental bacterial chromosome are separated by topoisomerases and are each paired with the complementary daughter strand.

B. Protein Synthesis

Protein synthesis is a multistep process which converts mRNA to the corresponding polypeptide chain (see, e.g., Michal, G. (1999) Biochemical Pathways: An Atlas of Biochemistry and Molecular Biology, Wiley: N.Y., and references therein; and Griffiths, A. J. F. et al., (1993) An Introduction to Genetic Analysis, 5^{th} ed., Freeman: N.Y. p. 391-398 and references therein). As the initiator codon (AUG, encoding methionine) first becomes accessible after being transcribed from the DNA by RNA polymerase, a translation initiation complex forms. This complex is comprised of the mRNA molecule itself, an initiation tRNA molecule (charged with methionine, corresponding to the first AUG codon of the mRNA molecule, and which has also been formylated to form the N-terminus of the nascent polypeptide), initiation factors, and the ribosome itself.

The bacterial ribosome (the 70S ribosome) contains two subunits. The first subunit is large (50S) while the second is small (30S). Each subunit contains a complex of RNA and protein molecules which assemble soon after or during their synthesis. These complexes are globular in shape, and the large subunit contains a long channel through which it is believed that the nascent polypeptide chain leaves the ribosome. There are at least three known sites of activity in the bacterial ribosome: one to bind a charged tRNA (aminoacyl tRNA), one to bind a tRNA associated with the nascent polypeptide chain, and the third to expel the uncharged tRNA from the complex. Ribosomes may occur singly or in groups, termed ‘polyribosomes’ or ‘polysomes’. These complexes are plentiful within the cell; one E. coli cell may contain as many as 15,000 ribosomes, constituting up to one quarter of the total biomass of the cell.

Upon the binding of the initiator tRNA^{met} in complex with the initiation factor IF-2 and GTP, the 30S ribosomal subunit binds such that the tRNA anticodon is associated with the peptidyl site in this molecule. The binding of the 50S subunit to this complex causes hydrolysis of the bound GTP, with concomitant release of the initiation factors. The amino-acid-charged tRNA corresponding to the second codon of the mRNA is positioned in the aminoacyl tRNA site in the ribosome (by the action of the elongation factor EF-Tu). The methionine attached to the tRNA in the peptidyl site and the amino acid bound to the tRNA in the aminoacyl site react to form a peptide bond, catalyzed by the peptidyltransferase activity of the 23S rRNA in the complex. Two simultaneous translocation steps subsequently occur in a GTP-dependent fashion: the nascent polypeptide-bound (peptidyl) tRNA remaining in the aminoacyl site is translocated to the peptidyl site of the ribosome (with concomitant displacement of the now uncharged tRNA in the peptidyl site to the ejection site), and the mRNA moves one codon site relative to the ribosome such that the next codon is exposed to the aminoacyl-tRNA site on the ribosome.

This cycle of amino acid addition and elongation of the peptide chain continues until a stop codon (UAA, UGA, UAG) is reached. There do not exist tRNA molecules specific for these stop codons; thus, no amino acid can be added. Instead, one of two release factors (specific to the particular codon in question) binds to the mRNA at the stop codon in a complex with release factor 3 and GTP. The release of the nascent polypeptide chain is accomplished by the hydrolysis of this GTP, and the remaining bound ribosomal subunits are dissociated through the activity of the ribosomal recycling factor.

C. Pathogenesis

Bacteria possess numerous mechanisms by which they are able to survive and even to adapt to environments with suboptimal growth conditions. These include protective elements (e.g., the cell wall, which prevents osmotic lysis), the ability to switch to the utilization of different nutrient sources (e.g., inorganic compounds, or carbon sources), and the ability to adjust to different stresses (e.g., temperature stress, osmotic stress, pH stress, or oxygen stress) by the activation of a sigma factor regulatory cascade. Under growth conditions in a complex environment containing cells other than the bacterium, many bacteria are capable of another survival mechanism: pathogenesis.

In order to survive in a host (e.g., a plant, animal, or human host), bacteria must be able to not only defend themselves against killing or removal by host immune systems, but also to proliferate. Many bacteria have developed multiple mechanisms by which each goal may be accomplished (see, e.g., Stanier et al. (1986) The Microbial World 5^{th} ed., Prentice Hall: Englewood Cliffs and references therein; and Hacker, J. (1999) “Prokaryotes in Medicine” in “Biology of the Prokaryotes, Lengeler et al., eds., Thieme Verlag: Stuttgart, p. 815-849, and references therein). Many bacteria produce peptide or protein toxins (e.g., hemolysins, or diphtheria toxin from Corynebacterium diphtheriae) which act to specifically or nonspecifically destroy host cells. Frequently these toxins are directed to immune cells which would otherwise act to remove the bacteria from the host. Such toxins may exert their lethal effect in a variety of ways, including by inhibition of protein synthesis in the target cell (e.g., exotoxin A from Pseudomonas aeruginosa or diphtheria toxin), by interfering with cellular signal, transduction in the target cell (e.g., anthrax lethal toxin, cholera toxin), or by simply creating holes in the target cell membrane which lead to cell lysis (e.g., hemolysins). These toxic activities manifest as a disease, for example, diphtheria, tuberculosis (Mycobacterium bovis or M. tuberculosis), anthrax (Bacillus anthracis).

Proliferation (i.e., colonization) of the bacterial cells depends on special factors termed adhesion factors or adhesins. These frequently proteinaceous molecules at the cell surface of the bacterium permit the bacterium to bind to one or more specific host cells or surfaces. This not only permits the bacterium to not be removed by circulatory and excretory processes, but it also limits the exposure of the bacterium to the host immune system, since the bacteria remain stationary and sometimes even inaccessible once adhered to a surface.

Corynebacterium glutamicum is a soil bacterium not known to be pathogenic, but its genome surprisingly includes several genes which are typically associated with bacterial pathogenesis, but the expression of which has never been observed. Similar situations have been observed in other bacteria: a bacterial species may have stains which are virulent (disease causing) and avirulent (nonpathogenic). A classic example of this is E. coli, from which both virulent (e.g., enteropathogenic species) and avirulent (e.g., K-12 strains) are well known. Certain bacteria are typically not pathogenic, but may still contain within their genome genes encoding proteins involved in pathogenicity, such as adhesins or toxins. These may be a evolutionary remnant, or may simply only be expressed under specific conditions which the bacterium rarely encounters.

III. Elements and Methods of the Invention

The present invention is based, at least in part, on the discovery of novel molecules, referred to herein as RRP nucleic acid and protein molecules, which participate in C. glutamicum DNA replication, protein synthesis, or pathogenesis. In one embodiment, the RRP molecules participate in the replication of C. glutamicum DNA, in C. glutamicum ribosome activity, or in the pathogenicity of the microorganism. In a preferred embodiment, the activity of the RRP molecules of the present invention with regard to DNA replication, protein synthesis, or pathogenesis has an impact on the production of a desired fine chemical by this organism. In a particularly preferred embodiment, the RRP molecules of the invention are modulated in activity, such that the C. glutamicum cellular processes in which the RRP molecules participate (e.g., DNA replication, protein synthesis, or pathogenesis) are also altered in activity, resulting either directly or indirectly in a modulation of the yield, production, and/or efficiency of production of a desired fine chemical by C. glutamicum.

The language, “RRP protein” or “RRP polypeptide” includes proteins which participate in a number of cellular processes related to C. glutamicum DNA replication, protein synthesis, or pathogenesis. For example, an RRP protein may be involved in the replication of C. glutamicum DNA, in C. glutamicum ribosome activity, or in the pathogenicity of the microorganism. Examples of RRP proteins include those encoded by the RRP genes set forth in Table 1 and Appendix A. The terms “RRP gene” or “RRP nucleic acid sequence” include nucleic acid sequences encoding an RRP protein, which consist of a coding region and also corresponding untranslated 5′ and 3′ sequence regions. Examples of RRP genes include those set forth in Table 1. The terms “production” or “productivity” are art-recognized and include the concentration of the fermentation product (for example, the desired fine chemical) formed within a given time and a given fermentation volume (e.g., kg product per hour per liter). The term “efficiency of production” includes the time required for a particular level of production to be achieved (for example, how long it takes for the cell to attain a particular rate of output of a fine chemical). The term “yield” or “product/carbon yield” is art-recognized and includes the efficiency of the conversion of the carbon source into the product (i.e., fine chemical). This is generally written as, for example, kg product per kg carbon source. By increasing the yield or production of the compound, the quantity of recovered molecules, or of useful recovered molecules of that compound in a given amount of culture over a given amount of time is increased. The terms “biosynthesis” or a “biosynthetic pathway” are art-recognized and include the synthesis of a compound, preferably an organic compound, by a cell from intermediate compounds in what may be a multistep and highly regulated process. The terms “degradation” or a “degradation pathway” are art-recognized and include the breakdown of a compound, preferably an organic compound, by a cell to degradation products (generally speaking, smaller or less complex molecules) in what may be a multistep and highly regulated process. The language “metabolism” is art-recognized and includes the totality of the biochemical reactions that take place in an organism. The metabolism of a particular compound, then, (e.g., the metabolism of an amino acid such as glycine) comprises the overall biosynthetic, modification, and degradation pathways in the cell related to this compound. The language “pathogenicity” or “pathogenesis” is art-recognized and includes the activity of an organism, such as a bacterial organism, to bring about a disease state in a host (e.g., cholera, diphtheria, or anthrax). Such disease states are typically the result of cell lytic activity of the organism, which may occur through the expression and release of cytotoxins (e.g., cholera toxin, diphtheria toxin, or anthrax toxin). Other bacterial proteins or peptides not pertaining specifically to cell lysis but contributing to the colonization of the host by the bacterium may also be considered pathogenesis proteins, such as, but not limited to, adhesins. The term “DNA replication” is art-recognized and includes all of the activities associated with the replication of DNA in vivo or in vitro, and for the purposes of the invention, particularly within bacterial cells. These activities include but are not limited to the assembly of DNA polymerases, the unwinding of DNA, the incorporation of new dNTPs into the nascent DNA strand, the excision and replacement of erroneous bases, and the termination of replication. The term “protein synthesis” is art-recognized and includes the process of converting mRNA codons into amino acids in a growing polypeptide chain, as catalyzed by the ribosome. The term “ribosome function” or “ribosome activity” is art-recognized and includes all of the functions of a ribosome, including, but not limited to, the binding of mRNA, the binding of an aminoacyl-tRNA and a peptidyl-tRNA, and the catalysis of the addition of the next amino acid to the growing polypeptide chain.

In another embodiment, the RRP molecules of the invention are capable of modulating the production of a desired molecule, such as a fine chemical, in a microorganism such as C. glutamicum. There are a number of mechanisms by which the alteration of an RRP protein of the invention may affect the yield, production, and/or efficiency of production of a fine chemical from a C. glutamicum strain incorporating such an altered protein. For example, by improving the rate at which DNA replication occurs (e.g., by optimizing the activity of one or more DNA polymerase, or by improving the rate at which the topoisomerases or helicases of the invention unwind DNA) it may be possible to increase the rate of cell division, which in turn increases the number of viable fine-chemical-producing C. glutamicum cells present in large-scale culture settings. Similarly, by improving the rate at which mRNA is translated to protein (e.g., by optimizing the activity of one or more of the ribosomal proteins) it may be possible to increase the number of proteins in the cell which participate in the synthesis of one or more desired fine chemicals, or in an overall increase in the rate of cell division (due to increased growth and metabolism), both of which should lead to increased production of one or more fine chemicals from large-scale fermentor cultures of these microorganisms. Alterations in the DNA replication proteins of the invention may also permit increased fidelity in the replicative process, thereby increasing the genetic stability and viability of the microorganism and lessening the chance that another engineered mutation improving fine chemical production will not be inadvertently mutagenized by error-prone replication. The RRP proteins of the invention involved in pathogenesis are themselves fine chemicals; by increasing the number or by engineering the corresponding genes such that the expression of these proteins is removed from cellular repression pathways, or by mutagenizing the proteins such that feedback regulatory regions are removed, it may be possible to increase the yield, production, and/or efficiency of production of these proteins from large-scale fermentor culture of organisms containing such mutations.

The isolated nucleic acid sequences of the invention are contained within the genome of a Corynebacterium glutamicum strain available through the American Type Culture Collection, given designation ATCC 13032. The, nucleotide sequence of the isolated C. glutamicum RRP DNAs and the predicted amino acid sequences of the C. glutamicum RRP proteins are shown in Appendices A and B, respectively. Computational analyses were performed which classified and/or identified these nucleotide sequences as sequences which encode proteins that participate in the replication of C. glutamicum DNA, in C. glutamicum ribosome activity, or in the pathogenicity of this microorganism.

The present invention also pertains to proteins which have an amino acid sequence which is substantially homologous to an amino acid sequence of Appendix B. As used herein, a protein which has an amino acid sequence which is substantially homologous to a selected amino acid sequence is least about 50% homologous to the selected amino acid sequence, e.g., the entire selected amino acid sequence. A protein which has an amino acid sequence which is substantially homologous to a selected amino acid sequence can also be least about 50-60%, preferably at least about 60-70%, and more preferably at least about 70-80%, 80-90%, or 90-95%, and most preferably at least about 96%, 97%, 98%, 99% or more homologous to the selected amino acid sequence.

The RRP protein or a biologically active portion or fragment thereof of the invention can participate in C. glutamicum DNA replication, in C. glutamicum protein synthesis, or in the pathogenicity of this microorganism, or have one or more of the activities set forth in Table 1.

Various aspects of the invention are described in further detail in the following subsections.

A. Isolated Nucleic Acid Molecules

One aspect of the invention pertains to isolated nucleic acid molecules that encode RRP polypeptides or biologically active portions thereof, as well as nucleic acid fragments sufficient for use as hybridization probes or primers for the identification or amplification of RRP-encoding nucleic acid (e.g., RRP DNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of the gene: at least about 100 nucleotides of sequence upstream from the 5′ end of the coding region and at least about 20 nucleotides of sequence downstream from the 3′ end of the coding region of the gene. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated RRP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g, a C. glutamicum cell). Moreover, an “isolated” nucleic acid molecule, such as a DNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having a nucleotide sequence of Appendix A, or a portion thereof, can be isolated using standard molecular biology techniques and the sequence information provided herein. For example, a C. glutamicum RRP DNA can be isolated from a C. glutamicum library using all or portion of one of the sequences of Appendix A as a hybridization probe and standard hybridization techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of one of the sequences of Appendix A can be isolated by the polymerase chain reaction using oligonucleotide primers designed based upon this sequence (e.g., a nucleic acid molecule encompassing all or a portion of one of the sequences of Appendix A can be isolated by the polymerase chain reaction using oligonucleotide primers designed based upon this same sequence of Appendix A). For example, mRNA can be isolated from normal endothelial cells (e.g., by the guanidinium-thiocyanate extraction procedure of Chirgwin et al. (1979) Biochemistry 18: 5294-5299) and DNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Appendix A. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to an RRP nucleotide sequence can be prepared by standard synthetic techniques, e.g., using an automated DNA synthesizer.

In a preferred embodiment, an isolated nucleic acid molecule of the invention comprises one of the nucleotide sequences shown in Appendix A. The sequences of Appendix A correspond to the Corynebacterium glutamicum RRP DNAs of the invention. This DNA comprises sequences encoding RRP proteins (i.e., the “coding region”, indicated in each sequence in Appendix A), as well as 5′ untranslated sequences and 3′ untranslated sequences, also indicated in Appendix A. Alternatively, the nucleic acid molecule can comprise only the coding region of any of the sequences in Appendix A.

For the purposes of this application, it will be understood that each of the sequences set forth in Appendix A has an identifying RXA or RXN number having the designation “RXA”, or “RXN” followed by 5 digits (i.e., RXA00823 or RXN00625). Each of these sequences comprises up to three parts: a 5′ upstream region, a coding region, and a downstream region. Each of these three regions is identified by the same RXA or RXN designation to eliminate confusion. The recitation “one of the sequences in Appendix A”, then, refers to any of the sequences in Appendix A, which may be distinguished by their differing RXA or RXN designations. The coding region of each of these sequences is translated into a corresponding amino acid sequence, which is set forth in Appendix B. The sequences of Appendix B are identified by the same RXA or RXN designations as Appendix A, such that they can be readily correlated. For example, the amino acid sequences in Appendix B designated RXA00823 and RXN00625 are translations of the coding regions of the nucleotide sequence of nucleic acid molecules RXA00823 and RXN00625, respectively, in Appendix A. Each of the RXA and RXN nucleotide and amino acid sequences of the invention has also been assigned a SEQ ID NO, as indicated in Table 1. For example, as set forth in Table 1, the nucleic acid sequence of RXAO1064 is SEQ ID NO:13, and the amino acid sequence of RXA01064 is SEQ ID NO:14.

Several of the genes of the invention are “F-designated genes”. An F-designated gene includes those genes set forth in Table 1 which have an ‘F’ in front of the RXA or RXN designation. For example, SEQ ID NO:3, designated, as indicated on Table 1, as “F RXA00625”, is an F-designated gene, as are SEQ ID NOs: 7, 17, and 25 (designated on Table 1 as “F RXA00538”, “F RXA01594”, and “F RXA00562”, respectively).

In one embodiment, the nucleic acid molecules of the present invention are not intended to include those compiled in Table 2. In the case of the dapD gene, a sequence for this gene was published in Wehrmann, A., et al. (1998) J. Bacteriol. 180(12): 3159-3165. However, the sequence obtained by the inventors of the present application is significantly longer than the published version. It is believed that the published version relied on an incorrect start codon, and thus represents only a fragment of the actual coding region.

In another preferred embodiment, an isolated nucleic acid molecule of the invention comprises a nucleic acid molecule which is a complement of one of the nucleotide sequences shown in Appendix A, or a portion thereof. A nucleic acid molecule which is complementary to one of the nucleotide sequences shown in Appendix A is one which is sufficiently complementary to one of the nucleotide sequences shown in Appendix A such that it can hybridize to one of the nucleotide sequences shown in Appendix A, thereby forming a stable duplex.

In still another preferred embodiment, an isolated nucleic acid molecule of the invention comprises a nucleotide sequence which is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to a nucleotide sequence shown in Appendix A, or a portion thereof. Ranges and identity values intermediate to the above-recited ranges, (e.g., 70-90% identical or 80-95% identical) are also intended to be encompassed by the present invention. For example, ranges of identity values using a combination of any of the above values recited as upper and/or lower limits are intended to be included. In an additional preferred embodiment, an isolated nucleic acid molecule of the invention comprises a nucleotide sequence which hybridizes, e.g., hybridizes under stringent conditions, to one of the nucleotide sequences shown in Appendix A, or a portion thereof.

Moreover, the nucleic acid molecule of the invention can comprise only a portion of the coding region of one of the sequences in Appendix A, for example a fragment which can be used as a probe or primer or a fragment encoding a biologically active portion of an RRP protein. The nucleotide sequences determined from the cloning of the RRP genes from C. glutamicum allows for the generation of probes and primers designed for use in identifying and/or cloning RRP homologues in other cell types and organisms, as well as RRP homologues from other Corynebacteria or related species. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense strand of one of the sequences set forth in Appendix A, an anti-sense sequence of one of the sequences set forth in Appendix A, or naturally occurring mutants thereof. Primers based on a nucleotide sequence of Appendix A can be used in PCR reactions to clone RRP homologues. Probes based on the RRP nucleotide sequences can be used to detect transcripts or genomic sequences encoding the same or homologous proteins. In preferred embodiments, the probe further comprises a label group attached thereto, e;g. the label group can be a radioisotope, a fluorescent compound, an enzyme, or an enzyme co-factor. Such probes can be used as a part of a diagnostic test kit for identifying cells which misexpress an RRP protein, such as by measuring a level of an RRP-encoding nucleic acid in a sample of cells, e.g., detecting RRP mRNA levels or determining whether a genomic RRP gene has been mutated or deleted.

In one embodiment, the nucleic acid molecule of the invention encodes a protein or portion thereof which includes an amino acid sequence which is sufficiently homologous to an amino acid sequence of Appendix B such that the protein or portion thereof maintains the ability to participate in the replication of C. glutamicum DNA, in C. glutamicum protein synthesis, or in the pathogenicity of this microorganism. As used herein, the language “sufficiently homologous” refers to proteins or portions thereof which have amino acid sequences which include a minimum number of identical or equivalent (e.g., an amino acid residue which has a similar side chain as an amino acid residue in one of the sequences of Appendix B) amino acid residues to an amino acid sequence of Appendix B such that the protein or portion thereof is able to participate in the replication of C. glutamicum DNA, in C. glutamicum protein synthesis, or in the pathogenicity of this microorganism. Proteins involved in C. glutamicum DNA replication, in ribosome function/activity, or in the pathogenesis of this microorganism, as described herein, may play a role in the production and secretion of one or more fine chemicals. Examples of such activities are also described herein. Thus, “the function of an RRP protein” contributes either directly or indirectly to the yield, production, and/or efficiency of production of one or more fine chemicals. Examples of RRP protein activities are set forth in Table 1.

In another embodiment, the protein is at least about 50-60%, preferably at least about 60-70%, and more preferably at least about 70-80%, 80-90%, 90-95%, and most preferably at least about 96%, 97%, 98%, 99% or more homologous to an entire amino acid sequence of Appendix B.

Portions of proteins encoded by the RRP nucleic acid molecules of the invention are preferably biologically active portions of one of the RRP proteins. As used herein, the term “biologically active portion of an RRP protein” is intended to include a portion, e.g., a domain/motif, of an RRP protein that can participate in the replication of C. glutamicum DNA, in C. glutamicum protein synthesis, or in the pathogenicity of this microorganism, or has an activity as set forth in Table 1. To determine whether an RRP protein or a biologically active portion thereof can participate in the replication of C. glutamicum DNA, in C. glutamicum protein synthesis, or in the pathogenicity of this microorganism, an assay of enzymatic/protein activity may be performed. Such assay methods are well known to those of ordinary skill in the art, as detailed in Example 8 of the Exemplification.

Additional nucleic acid fragments encoding biologically active portions of an RRP protein can be prepared by isolating a portion of one of the sequences in Appendix B, expressing the encoded portion of the RRP protein or peptide (e.g., by recombinant expression in vitro) and assessing the activity of the encoded portion of the RRP protein or peptide.

The invention further encompasses nucleic acid molecules that differ from one of the nucleotide sequences shown in Appendix A (and portions thereof) due to degeneracy of the genetic code and thus encode the same RRP protein as that encoded by the nucleotide sequences shown in Appendix A. In another embodiment, an isolated nucleic acid molecule of the invention has a nucleotide sequence encoding a protein having an amino acid sequence shown in Appendix B. In a still further embodiment, the nucleic acid molecule of the invention encodes a full length C. glutamicum protein which is substantially homologous to an amino acid sequence of Appendix B (encoded by an open reading frame shown in Appendix A).

It will be understood by one of ordinary skill in the art that in one embodiment the sequences of the invention are not meant to include the sequences of the prior art, such as those Genbank sequences set forth in Tables 2 or 4 which were available prior to the present invention. In one embodiment, the invention includes nucleotide and amino acid sequences having a percent identity to a nucleotide or amino acid sequence of the invention which is greater than that of a sequence of the prior art (e.g., a Genbank sequence (or the protein encoded by such a sequence) set forth in Tables 2 or 4). For example, the invention includes a nucleotide sequence which is greater than and/or at least 38% identical to the nucleotide sequence designated RXA00823 (SEQ ID NO:9), a nucleotide sequence which is greater than and/or at least 40% identical to the nucleotide sequence designated RXA01064 (SEQ ID NO:13), and a nucleotide sequence which is greater than and/or at least 45% identical to the nucleotide sequence designated RXA02363 (SEQ ID NO:35). One of ordinary skill in the art would be able to calculate the lower threshold of percent identity for any given sequence of the invention by examining the GAP-calculated percent identity scores set forth in Table 4 for each of the three top hits for the given sequence, and by subtracting the highest GAP-calculated percent identity from 100 percent. One of ordinary skill in the art will also appreciate that nucleic acid and amino acid sequences having percent identities greater than the lower threshold so calculated (e.g., at least 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more identical) are also encompassed by the invention.

In addition to the C. glutamicum RRP nucleotide sequences shown in Appendix A, it will be appreciated by those of ordinary skill in the art that DNA sequence polymorphisms that lead to changes in the amino acid sequences of RRP proteins may exist within a population (e.g., the C. glutamicum population). Such genetic polymorphism in the RRP gene may exist among individuals within a population due to natural variation. As used herein, the terms “gene” and “recombinant gene” refer to nucleic acid molecules comprising an open reading frame encoding an RRP protein, preferably a C. glutamicum RRP protein. Such natural variations can typically result in 1-5% variance in the nucleotide sequence of the RRP gene. Any and all such nucleotide variations and resulting amino acid polymorphisms in RRP that are the result of natural variation and that do not alter the functional activity of RRP proteins are intended to be within the scope of the invention.

Nucleic acid molecules corresponding to natural variants and non-C. glutamicum homologues of the C. glutamicum RRP DNA of the invention can be isolated based on their homology to the C. glutamicum RRP nucleic acid disclosed herein using the C. glutamicum DNA, or a portion thereof, as a hybridization probe according to standard hybridization techniques under stringent hybridization conditions. Accordingly, in another embodiment, an isolated nucleic acid molecule of the invention is at least 15 nucleotides in length and hybridizes under stringent conditions to the nucleic acid molecule comprising a nucleotide sequence of Appendix A. In other embodiments, the nucleic acid is at least 30, 50, 100, 250 or more nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those of ordinary skill in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to a sequence of Appendix A corresponds to a naturally-occurring nucleic acid molecule. As used herein, a “naturally occurring” nucleic acid molecule refers to an RNA or DNA molecule having a nucleotide sequence that occurs in nature (e.g., encodes a natural protein). In one embodiment, the nucleic acid encodes a natural C. glutamicum RRP protein.

In addition to naturally-occurring variants of the RRP, sequence that may exist in the population, the one of ordinary skill in the art will further appreciate that changes can be introduced by mutation into a nucleotide sequence of Appendix A, thereby leading to changes in the amino acid sequence of the encoded RRP protein, without altering the functional ability of the RRP protein. For example, nucleotide substitutions leading to amino acid substitutions at “non-essential” amino acid residues can be made in a sequence of Appendix A. A “non-essential” amino acid residue is a residue that can be altered from the wild-type sequence of one of the RRP proteins (Appendix B) without altering the activity of said RRP protein, whereas an “essential” amino acid residue is required for RRP protein activity. Other amino acid residues, however, (e.g., those that are not conserved or only semi-conserved in the domain having RRP activity) may not be essential for activity and thus are likely to be amenable to alteration without altering RRP activity.

Accordingly, another aspect of the invention pertains to nucleic acid molecules encoding RRP proteins that contain changes in amino acid residues that are not essential for RRP activity. Such RRP proteins differ in amino acid sequence from a sequence contained in Appendix B yet retain at least one of the RRP activities described herein. In one embodiment, the isolated nucleic acid molecule comprises a nucleotide sequence encoding a protein, wherein the protein comprises an amino acid sequence at least about 50% homologous to an amino acid sequence of Appendix B and is capable of participating in the replication of C. glutamicum DNA, in C. glutamicum protein synthesis, or in the pathogenicity of this microorganism, or has one or more of the activities set forth in Table 1. Preferably, the protein encoded by the nucleic acid molecule is at least about 50-60% homologous to one of the sequences in Appendix B, more preferably at least about 60-70% homologous to one of the sequences in Appendix B, even more preferably at least about 70-80%, 80-90%, 90-95% homologous to one of the sequences in Appendix B, and most preferably at least about 96%, 97%, 98%, or 99% homologous to one of the sequences in Appendix B.

To determine the percent homology of two amino acid sequences (e.g., one of the sequences of Appendix B and a mutant form thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the sequences of Appendix B) is occupied by the same amino acid residue or nucleotide as the corresponding position in the other sequence (e.g., a mutant form of the sequence selected from Appendix B), then the molecules are homologous at that position (i.e., as used herein amino acid or nucleic acid “homology” is equivalent to amino acid or nucleic acid. “identity”). The percent homology between the two sequences is a function of the number of identical positions shared by the sequences (i.e., % homology=# of identical positions/total # of positions×100).

An isolated nucleic acid molecule encoding an RRP protein homologous to a protein sequence of Appendix B can be created by introducing one or more nucleotide substitutions, additions or deletions into a nucleotide sequence of Appendix A such that one or more amino acid substitutions, additions or deletions are introduced into the encoded protein. Mutations can be introduced into one of the sequences of Appendix A by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an RRP protein is preferably replaced with another amino acid residue from the same side chain family. Alternatively, in another embodiment, mutations can be introduced randomly along all or part of an RRP coding sequence, such as by saturation mutagenesis, and the resultant mutants can be screened for an RRP activity described herein to identify mutants that retain RRP activity. Following mutagenesis of one of the sequences of Appendix A, the encoded protein can be expressed recombinantly and the activity of the protein can be determined using, for example, assays described herein (see Example 8 of the Exemplification).

In addition to the nucleic acid molecules encoding RRP proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded DNA molecule or complementary to an RNA sequences. Accordingly, an antisense nucleic acid can hydrogen bond to a senses nucleic acid. The antisense nucleic acid can be complementary to an entire RRP coding strand, or to only a portion thereof. In one embodiment, an antisense nucleic acid molecule is antisense to a “coding region” of the coding strand of a nucleotide sequence encoding an RRP protein. The term “coding region” refers to the region of the nucleotide sequence comprising codons which are translated into amino acid residues (e.g., the entire coding region of SEQ ID NO:5 (RXN02943) comprises nucleotides 1 to 1668). In another embodiment, the antisense nucleic acid molecule is antisense to a “noncoding region” of the coding strand of a nucleotide sequence encoding RRP. The term “noncoding region” refers to 5′ and 3′ sequences which flank the coding region that are not translated into amino acids (i.e., also referred to as 5′ and 3′ untranslated regions).

Given the coding strand sequences encoding RRP disclosed herein (e.g., the sequences set forth in Appendix A), antisense nucleic acids of the invention can be designed according to the rules of Watson and Crick base pairing. The antisense nucleic acid molecule can be complementary to the entire coding region of RRP mRNA, but more preferably is an oligonucleotide which is antisense to only a portion of the coding or noncoding region of RRP mRNA. For example, the antisense oligonucleotide can be complementary to the region surrounding the translation start site of RRP mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v); 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a cell or generated in situ such that they hybridize with or bind to cellular mRNA and/or genomic DNA encoding an RRP protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave RRP mRNA transcripts to thereby inhibit translation of RRP mRNA. A ribozyme having specificity for an RRP-encoding nucleic acid can be designed based upon the nucleotide sequence of an RRP DNA molecule disclosed herein (i.e., SEQ ID NO:9 (RXA00823 in Appendix A)). For example, a derivative of a Tetrahymena L-19 IVS RNA can be constructed in which the nucleotide sequence of the active site is complementary to the nucleotide sequence to be cleaved in an RRP-encoding mRNA. See, e.g., Cech et al. U.S. Pat. No. 4,987,071 and Cech et al. U.S. Pat. No. 5,116,742. Alternatively, RRP mRNA can bemused tobselect acatalytic RNA having a specific ribonuclease activity from a pool of RNA molecules. See, e.g., Bartel, D. and Szostak, J. W. (1993) Science 261:1411-1418.

Alternatively, RRP gene expression can be inhibited by targeting nucleotide sequences complementary to the regulatory region of an RRP nucleotide sequence (e.g., an RRP promoter and/or enhancers) to form triple helical structures that prevent transcription of an RRP gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

B. Recombinant Expression Vectors and Host Cells

Another aspect of the invention pertains to vectors, preferably expression vectors, containing a nucleic acid encoding an RRP protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which are operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells. Preferred regulatory sequences are, for example, promoters such as cos-, tac-, trp-, tet-, trp-tet-, lpp-, lac-, lpp-lac-, laclI^{q}-, T7-, T5-, T3-, gal-, trc-, ara-, SP6-, amy, SPO2, λ-P_{R}- or λP_{L}, which are used preferably in bacteria. Additional regulatory sequences are, for example, promoters from yeasts and fungi, such as ADC1, MFα, AC, P-60, CYC1, GAPDH, TEF, rp28, ADH, promoters from plants such as CaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., RRP proteins, mutant forms of RRP proteins, fusion proteins, etc.).

The recombinant expression vectors of the invention can be designed for expression of RRP proteins in prokaryotic or eukaryotic cells. For example, RRP genes can be expressed in bacterial cells such as C. glutamicum, insect cells (using baculovirus expression vectors), yeast and other fungal cells (see Romanos, M. A. et al. (1992) “Foreign gene expression in yeast: a review”, Yeast 8: 423-488; van den Hondel, C. A. M. J. J. et al. (1991) “Heterologous gene expression in filamentous fungi” in: More Gene Manipulations in Fungi, J. W. Bennet & L. L. Lasure, eds., p. 396-428: Academic Press: San Diego; and van den Hondel, C. A. M. J. J. & Punt, P. J. (1991) “Gene transfer systems and vector development for filamentous fungi, in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) High efficiency Agrobacterium tumefaciens—mediated transformation of Arabidopsis thaliana leaf and cotyledon explants” Plant Cell Rep.: 583-586), or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein but also to the C-terminus or fused within suitable regions in the proteins. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the RRP protein is cloned into a pGEX expression vector to create a vector encoding a fusion protein comprising, from the N-terminus to the C-terminus, GST-thrombin cleavage site-X protein. The fusion protein can be purified by affinity chromatography using glutathione-agarose resin. Recombinant RRP protein unfused to GST can be recovered by cleavage of the fusion protein with thrombin.

Examples of suitable inducible non-fusion E. coli expression vectors include pTrc (Amann et al., (1988) Gene 69:301-315) pLG338, pACYC184, pBR322, pUC18, pUC19, pKC30, pRep4, pHS1, pHS2, pPLc236, pMBL24, pLG200, pUR290, pIN-III113-B1, λgt11, pBdCl, and pET 11d (Studier et al., Gene Expression Technology: Methods in Enzymology 185, Academic Press, San Diego, Calif. (1990) 60-89; and Pouwels et al., eds. (1985) Cloning Vectors. Elsevier: New York IBSN 0 444 904018). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS174(DE3) from a resident λ prophage harboring a T7 gn1 gene under the transcriptional control of the lacUV 5 promoter. For transformation of other varieties of bacteria, appropriate vectors may be selected. For example, the plasmids pIJ101, pIJ364, pIJ702 and pIJ361 are known to be useful in transforming Streptomyces, while plasmids pUB110, pC194, or pBD214 are suited for transformation of Bacillus species. Several plasmids of use in the transfer of genetic information into Corynebacterium include pHM1519, pBL1, pSA77, or pAJ667 (Pouwels et al., eds. (1985) Cloning Vectors. Elsevier: New York IBSN 0 444 904018). One strategy to maximize recombinant protein expression is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in the bacterium chosen for expression, such as C. glutamicum (Wada et al. (1992) Nucleic Acids Res. 20: 2111-2118). Such alteration of nucleic acid sequences of the invention can be carried out by standard DNA synthesis techniques.

In another embodiment, the RRP protein expression vector is a yeast expression vector. Examples of vectors for expression in yeast S. cerevisiae include pYepSec1 (Baldari, et al., (1987) Embo J. 6:229-234), 2 μ, pAG-1, Yep6, Yepl3, pEMBLYe23, pMFa (Kurjan and Herskowitz, (1982) Cell 30:933-943), pJRY88 (Schultz et al., (1987) Gene 54:113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge, and Pouwels et al., eds. (1985) Cloning Vectors. Elsevier: N.Y. (IBSN 0 444 904018).

Alternatively, the RRP proteins of the invention can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In another embodiment, the RRP proteins of the invention may be expressed in unicellular plant cells (such as algae) or in plant cells from higher plants (e.g., the spermatophytes, such as crop plants). Examples of plant expression vectors include those detailed in: Becker, D., Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation”, Nucl. Acid. Res. 12: 8711-8721, and include pLGV23, pGHlac+, pBIN19, pAK2004, and pDH51 (Pouwels et al., eds. (1985) Cloning Vectors. Elsevier: N.Y. IBSN 0 444 904018). In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufinan et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Baneiji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the cc-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to RRP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) (1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an RRP protein can be expressed in bacterial cells such as C. glutamicum, insect cells, yeast or mammalian cells (such as Chinese hamster ovary cells (CHO) or COS cells). Other suitable host cells are known to one of ordinary skill in the art. Microorganisms related to Corynebacterium glutamicum which may be conveniently used as host cells for the nucleic acid and protein molecules of the invention are set forth in Table 3.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, phasmid, phagemid, transposon or other DNA) into a host cell, including calcium phosphate or calcium chloride co-precipitation, DEAE-dextran-mediated transfection, lipofection, natural competence, chemical-mediated transfer, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an RRP protein or can be introduced on a separate vector. Cells stably transfected with the introduced nucleic acid can be identified by, for example, drug selection (e.g., cells that have incorporated the selectable marker gene will survive, while the other cells die).

To create a homologous recombinant microorganism, a vector is prepared which contains at least a portion of an RRP gene into which a deletion, addition or substitution has been introduced to thereby alter, e.g., functionally disrupt, the RRP gene. Preferably, this RRP gene is a Corynebacterium glutamicum RRP gene, but it can be a homologue from a related bacterium or even from a mammalian, yeast, or insect source In a preferred embodiment, the vector is designed such that, upon homologous recombination, the endogenous RRP gene is functionally-disrupted (i.e., no longer encodes a functional protein; also referred to as a “knock out” vector). Alternatively, the vector can be designed such that, upon homologous recombination, the endogenous RRP gene is mutated or otherwise altered but still encodes functional protein (e.g., the upstream regulatory region can be altered to thereby alter the expression of the endogenous RRP protein). In the homologous recombination vector, the altered portion of the RRP gene is flanked at its 5′ and 3′ ends by additional nucleic acid of the RRP gene to allow for homologous recombination to occur between the exogenous RRP gene carried by the vector and an endogenous RRP gene in a microorganism. The additional flanking RRP nucleic acid is of sufficient length for successful homologous recombination with the endogenous gene. Typically, several kilobases of flanking DNA (both at the 5′ and 3′ ends) are included in the vector (see e.g., Thomas, K. R., and Capecchi, M. R. (1987) Cell 51: 503 for a description of homologous recombination vectors). The vector is introduced into a microorganism (e.g., by electroporation) and cells in which the introduced RRP gene has homologously recombined with the endogenous RRP gene are selected, using art-known techniques.

In another embodiment, recombinant microorganisms can be produced which contain selected systems which allow for regulated expression of the introduced gene. For example, inclusion of an RRP gene on a vector placing it under control of the lac operon permits expression of the RRP gene only in the presence of IPTG. Such regulatory systems are well known in the art.

In another embodiment, an endogenous RRP gene in a host cell is disrupted (e.g., by homologous recombination or other genetic means known in the art) such that expression of its protein product does not occur. In another embodiment, an endogenous or introduced RRP gene in a host cell has been altered by one or more point mutations, deletions, or inversions, but still encodes a functional RRP protein. In still another embodiment, one or more of the regulatory regions (e.g., a promoter, repressor, or inducer) of an RRP gene in a microorganism has been altered (e.g., by deletion, truncation, inversion, or point mutation) such that the expression of the RRP gene is modulated. One of ordinary skill in the art will appreciate that host cells containing more than one of the described RRP gene and protein modifications may be readily produced using the methods of the invention, and are meant to be included in the present invention.

A host cell of the invention, such as a prokaryotic or eukaryotic host cell in culture, can be used to produce (i.e., express) an RRP protein. Accordingly, the invention further provides methods for producing RRP proteins using the host cells of the invention. In one embodiment, the method comprises culturing the host cell of invention (into which a recombinant expression vector encoding an RRP protein has been introduced, or into which genome has been introduced a gene encoding a wild-type or altered RRP protein) in a suitable medium until RRP protein is produced. In another embodiment, the method further comprises isolating RRP proteins from the medium or the host cell.

C. Isolated RRP Proteins

Another aspect of the invention pertains to isolated RRP proteins, and biologically active portions thereof. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of RRP protein in which the protein is separated from cellular components of the cells in which it is naturally or recombinantly produced. In one embodiment, the language “substantially free of cellular material” includes preparations of RRP protein having less than about 30% (by dry weight) of non-RRP protein (also referred to herein as a “contaminating protein”), more preferably less than about 20% of non-RRP protein, still more preferably less than about 10% of non-RRP protein, and most preferably less than about 5% non-RRP protein. When the RRP protein or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of RRP protein in which the protein is separated from chemical precursors or other chemicals which are involved in the synthesis of the protein. In one embodiment, the language “substantially free of chemical precursors or other chemicals” includes preparations of RRP protein having less than about 30% (by dry weight) of chemical precursors or non-RRP chemicals, more preferably less than about 20% chemical precursors or non-RRP chemicals, still more preferably less than about 10% chemical precursors or non-RRP chemicals, and most preferably less than about 5% chemical precursors or non-RRP chemicals. In preferred embodiments, isolated proteins or biologically active portions thereof lack contaminating proteins from the same organism from which the RRP protein is derived. Typically, such proteins are produced by recombinant expression of, for example, a C. glutamicum RRP protein in a microorganism such as C. glutamicum.

An isolated RRP protein or a portion thereof of the invention can participate in the replication of C. glutamicum DNA, in C. glutamicum protein synthesis, or in the pathogenicity of this microorganism, or has one or more of the activities set forth in Table 1. In preferred embodiments, the protein orportion thereof comprises an amino acid sequence which is sufficiently homologous to an amino acid sequence of Appendix B such that the protein or portion thereof maintains the ability to participate in the replication of C. glutamicum DNA, in C. glutamicum protein synthesis, or in the pathogenicity of this microorganism. The portion of the protein is preferably a biologically active portion as described herein. In another preferred embodiment, an RRP protein of the invention has an amino acid sequence shown in Appendix B. In yet another preferred embodiment, the RRP protein has an amino acid sequence which is encoded by a nucleotide sequence which hybridizes, e.g., hybridizes under stringent conditions, to a nucleotide sequence of Appendix A. In still another preferred embodiment, the RRP protein has an amino acid sequence which is encoded by a nucleotide sequence that is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to one of the nucleic acid sequences of Appendix A, or a portion thereof. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. The preferred RRP proteins of the present invention also preferably possess at least one of the RRP activities described herein. For example, a preferred RRP protein of the present invention includes an amino acid sequence encoded by a nucleotide sequence which hybridizes, e.g., hybridizes under stringent conditions, to a nucleotide sequence of Appendix A, and which can participate in the replication of C. glutamicum DNA, in C. glutamicum protein synthesis, or in the pathogenicity of this microorganism, or which has one or more of the activities set forth in Table 1.

In other embodiments, the RRP protein is substantially homologous to an amino acid sequence of Appendix B and retains the functional activity of the protein of one of the sequences of Appendix B yet differs in amino acid sequence due to natural variation or mutagenesis, as described in detail in subsection I above. Accordingly, in another embodiment, the RRP protein is a protein which comprises an amino acid sequence which is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%; 99% or more homologous to an entire amino acid sequence of Appendix B and which has at least one of the RRP activities described herein. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. In another embodiment, the invention pertains to a full length C. glutamicum protein which is substantially homologous to an entire amino acid sequence of Appendix B.

Biologically active portions of an RRP protein include peptides comprising amino acid sequences derived from the amino acid sequence of an RRP protein, e.g., an amino acid sequence shown in Appendix B or the amino acid sequence of a protein homologous to an RRP protein, which include fewer amino acids than a full length RRP protein or the full length protein which is homologous to an RRP protein, and exhibit at least one activity of an RRP protein. Typically, biologically active portions (peptides, e.g., peptides which are, for example, 5, 10, 15, 20, 30, 35, 36, 37, 38, 39, 40, 50, 100 or more amino acids in length) comprise a domain or motif with at least one activity of an RRP protein. Moreover, other biologically active portions, in which other regions of the protein are deleted, can be prepared by recombinant techniques and evaluated for one or more of the activities described herein. Preferably, the biologically active portions of an RRP protein include one or more selected domains/motifs or portions thereof having biological activity.

RRP proteins are preferably produced by recombinant DNA techniques. For example, a nucleic acid molecule encoding the protein is cloned into an expression vector (as described above), the expression vector is introduced into a host cell (as described above) and the RRP protein is expressed in the host cell. The RRP protein can then be isolated from the cells by an appropriate purification scheme using standard protein purification techniques. Alternative to recombinant expression, an RRP protein, polypeptide, or peptide can be synthesized chemically using standard peptide synthesis techniques. Moreover, native RRP protein can be isolated from cells (e.g., endothelial cells), for example using an anti-RRP antibody, which can be produced by standard techniques utilizing an RRP protein or fragment thereof of this invention.

The invention also provides RRP chimeric or fusion proteins. As used herein, an RRP “chimeric protein” or “fusion protein” comprises an RRP polypeptide operatively linked to a non-RRP polypeptide. An “RRP polypeptide” refers to a polypeptide having an amino acid sequence corresponding to an RRP protein, whereas a “non-RRP polypeptide” refers to a polypeptide having an amino acid sequence corresponding to a protein, which is not substantially homologous to the RRP protein, e.g., a protein which is different from the RRP protein and which is derived from thesame or a different organism. Within the fusion protein, the term “operatively linked” is intended to indicate that the RRP polypeptide and the non-RRP polypeptide are fused in-frame to each other. The non-RRP polypeptide can be fused to the N-terminus or C-terminus of the RRP polypeptide. For example, in one embodiment the fusion protein is a GST-RRP, fusion protein in which the RRP sequences are fused to the C-terminus of the GST sequences. Such fusion proteins can facilitate the purification of recombinant RRP proteins. In another embodiment, the fusion protein is an RRP protein containing a heterologous signal sequence at its N-terminus. In certain host cells (e.g., mammalian host cells), expression and/or secretion of an RRP protein can be increased through use of a heterologous signal sequence.

Preferably, an RRP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An RRP-encoding nucleic acid can be cloned into such an expression vector such that the fusion moiety is linked in-frame to the RRP protein.

Homologues of the RRP protein can be generated by mutagenesis, e.g., discrete point mutation or truncation of the RRP protein. As used herein, the term “homologue” refers to a variant form of the RRP protein which acts as an agonist or antagonist of the activity of the RRP protein. An agonist of the RRP protein can retain substantially the same, or a subset, of the biological activities of the RRP protein. An antagonist of the RRP protein can inhibit one or more of the activities of the naturally occurring form of the RRP protein, by, for example, competitively binding to a downstream or upstream member of a biochemical cascade which includes the RRP protein, by binding to a target molecule with which the RRP protein interacts, such that no functional interaction is possible, or by binding directly to the RRP protein and inhibiting its normal activity.

In an alternative embodiment, homologues of the RRP protein can be identified by screening combinatorial libraries of mutants, e.g., truncation mutants, of the RRP protein for RRP protein agonist or antagonist activity. In one embodiment, a variegated library of RRP variants is generated by combinatorial mutagenesis at the nucleic acid level and is encoded by a variegated gene library. A variegated library of RRP variants can be produced by, for example, enzymatically ligating a mixture of synthetic oligonucleotides into gene sequences such that a degenerate set of potential RRP sequences is expressible as individual polypeptides, or alternatively, as a set of larger fusion proteins (e.g., for phage display) containing the set of RRP sequences therein. There are a variety of methods which can be used to produce libraries of potential RRP homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential RRP sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the RRP protein coding can be used to generate a variegated population of RRP fragments for screening and subsequent selection of homologues of an RRP protein. In one embodiment, a library of coding sequence fragments can be generated by treating a double stranded PCR fragment of an RRP coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the RRP protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of RRP homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify RRP homologues (Arkin and Yourvan (1992) PNAS 89:7811-7815; Delgrave et al. (1993) Protein Engineering 6(3):327-331).

In another embodiment, cell based assays can be exploited to analyze a variegated RRP library, using methods well known in the art.

D. Uses and Methods of the Invention

The nucleic acid molecules, proteins, protein homologues, fusion proteins, primers, vectors, and host cells described herein can be used in one or more of the following methods: identification of C. glutamicum and related organisms; mapping of genomes of organisms related to C. glutamicum; identification and localization of C. glutamicum sequences of interest; evolutionary studies; determination of RRP protein regions required for function; modulation of an RRP protein activity; modulation of DNA synthesis; modulation of protein synthesis; modulation of C. glutamicum pathogenesis; and modulation of cellular production of a desired compound, such as a fine chemical.

The RRP nucleic acid molecules of the invention have a variety of uses. First, they may be used to identify an organism as being Corynebacterium glutamicum or a close relative thereof. Also, they may be used to identify the presence of C. glutamicum or a relative thereof in a mixed population of microorganisms. The invention provides the nucleic acid sequences of a number of C. glutamicum genes; by probing the extracted genomic DNA of a culture of a unique or mixed population of microorganisms under stringent conditions with a probe spanning a region of a C. glutamicum gene which is unique to this organism, one can ascertain whether this organism is present.

Although Corynebacterium glutamicum itself is not pathogenic in humans, it is related to species which are human pathogens, such as Corynebacterium diphtheriae. Corynebacterium diphtheriae is the causative agent of diphtheria, a rapidly developing, acute, febrile infection which involves both local and systemic pathology. In this disease, a local lesion develops in the upper respiratory tract and involves necrotic injury to epithelial cells; the bacilli secrete toxin which is disseminated through this lesion to distal susceptible tissues of the body. Degenerative changes brought about by the inhibition of protein synthesis in these tissues, which include heart, muscle, peripheral nerves, adrenals, kidneys, liver and spleen, result in the systemic pathology of the disease. Diphtheria continues to have high incidence in many parts of the world, including Africa, Asia, Eastern Europe and the independent states of the former Soviet Union. An ongoing epidemic of diphtheria in the latter two regions has resulted in at least 5,000 deaths since 1990.

In one embodiment, the invention provides a method of identifying the presence or activity of Cornyebacterium diphtheriae in a subject. This method includes detection of one or more of the nucleic acid or amino acid sequences of the invention (e.g., the sequences set forth in Appendix A or Appendix B) in a subject, thereby detecting the presence or activity of Corynebacterium diphtheriae in the subject. C. glutamicum and C. diphtheriae are related bacteria, and many of the nucleic acid and protein molecules in C. glutamicum are homologous to C. diphtheriae nucleic acid and protein molecules, and can therefore be used to detect C. diphtheriae in a subject.

The nucleic acid and protein molecules of the invention may also serve as markers for specific regions of the genome. This has utility not only in the mapping of the genome, but also for functional studies of C. glutamicum proteins. For example, to identify the region of the genome to which a particular C. glutamicum DNA-binding protein binds, the C. glutamicum genome could be digested, and the fragments incubated with the DNA-binding protein. Those which bind the protein may be additionally probed with the nucleic acid molecules of the invention, preferably with readily detectable labels; binding of such a nucleic acid molecule to the genome fragment enables the localization of the fragment to the genome map of C. glutamicum, and, when performed multiple times with different enzymes, facilitates a rapid determination of the nucleic acid sequence to which the protein binds. Further, the nucleic acid molecules of the invention may be sufficiently homologous to the sequences of related species such that these nucleic acid molecules may serve as markers for the construction of a genomic map in related bacteria, such as Brevibacterium lactofermentum.

The RRP nucleic acid molecules encoding proteins involved in the pathogenicity of C. glutamicum are also useful for purposes of genetic engineering of this microorganism. Frequently, the insertion of genetic information into the genome of an organism is a disruptive process, which may inadvertently impair the regulation or coding regions of multiple different genes. The RRP pathogenicity genes of the invention are not necessary for the continued survival of the organism in an artificial culture setting, and are not likely to add any benefit to the productivity of the organism for one or more fine chemicals. These genes, then, may serve as useful insertion points for the addition of genetic material to the genome of C. glutamicum, since their disruption should not affect the viability or the productivity of this microorganism.

The RRP nucleic acid molecules of the invention are also useful for evolutionary and protein structural studies. The processes involved in DNA replication, protein synthesis and pathogenesis in which the molecules of the invention participate are utilized by a wide variety of species; by comparing the sequences of the nucleic acid molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Manipulation of the RRP nucleic acid molecules of the invention may result in the production of RRP proteins having functional differences from the wild-type RRP proteins. These proteins may be improved in efficiency or activity, may be present in greater numbers in the cell than is usual, or may be decreased in efficiency or activity.

The invention provides methods for screening molecules which modulate the activity of an RRP protein, either by interacting with the protein itself or a substrate or binding partner of the RRP protein, or by modulating the transcription or translation of an RRP nucleic acid molecule of the invention. In such methods, a microorganism expressing one or more RRP proteins of the invention is contacted with one or more test compounds, and the effect of each test compound on the activity or level of expression of the RRP protein is assessed.

The alteration of activity or number of one or more of the RRP proteins of the invention involved in DNA replication may affect fine chemical production from a C. glutamicum (or related bacterial) cell containing such mutations. For example, by improving the rate at which a DNA polymerase of the invention synthesizes DNA, it may be possible to improve the overall replication rate of the genomic DNA. Similarly, by optimizing the activity of the topoisomerases or gyrases of the invention, it may be possible to more quickly unwind the DNA, thereby permitting increased progressivity of the polymerase complex along the bacterial chromosome. Further, it may be possible to engineer one or more of the proteins involved in DNA replication such that they are improved for function under conditions of high temperature and nonoptimal pH, such as those found in large-scale fermentor culture (e.g., amino acid replacements may be made such that the resulting structure of one of these proteins retains activity but is improved for stability at high temperature or acidic/basic pH). Improving the rate of DNA synthesis in C. glutamicum or related bacteria may permit more rapid rates of cell division, leading to increased numbers of cells present in large-scale cultures of the bacterium. Relatively increased numbers of cells, each of which is producing one or more desired fine chemicals, should result in relatively increased yield, production, or efficiency of production of one or more fine chemicals from the culture.

Also, by manipulating one or more of the RRP genes of the invention, it may be possible to increase the overall fidelity of replication in C. glutamicum or related bacterial cells. For example, the 3,′-5′ exonuclease activities of Pol III or Pol I (which are responsible for excising inappropriately incorporated bases from the growing DNA strand) may be optimized such that more incorrect bases are detected and removed. Similarly, the polymerization activity of the DNA polymerases of the invention may be improved such that the error rate in base incorporation is decreased. Both such modifications should result in improved fidelity in the replicated DNA, which in turn should decrease the rate of introduced mutations. Fewer introduced mutations not only helps to ensure that any other engineered genes will not be undesirably altered by random mutation, but also may permit increased viability of the cells in culture, since random mutations may impair the activity of genes necessary for cell survival. As before, increased numbers of viable cells in culture should result in increased yield, production, and/or efficiency of production of one or more fine chemicals produced by those cells.

Mutations in genes and proteins involved in protein synthesis (e.g., ribosomal genes and proteins) may also have a significant effect on the production of one or more fine chemicals from C. glutamicum or related bacterial cultures. For example, by improving the rate of protein synthesis (e.g., by improving the rate of assembly of the ribosome, by improving the progressivity of the ribosome, or by increasing the rate at which the ribosome is able to productively bind to mRNA, all of which may be accomplished by altering the binding sites for the various ribosomal components such that binding and association of ribosomal proteins to each other or to tRNAs or to mRNAs are improved) it may be possible to increase the rate at which proteins involved in the synthesis of desired fine chemicals are produced, thereby potentially improving the production of one or more of these fine chemicals. This increased protein production may also permit increased growth and cell division of the cell, since increased cellular metabolism (which may occur due to the presence of increased numbers of metabolic proteins) may also result in more rapid cell division, thereby increasing the number of cells in a culture of the bacterium containing such mutation(s). Increased numbers of viable cells in large-scale fermentor culture, each of which is producing one or more desired fine chemicals, should result in an increase in yield, production, and/or efficiency of production of these fine chemicals.

Alteration of the number of the RRP proteins of the invention involved in the pathogenicity of C. glutamicum (e.g., hemolysin and invasin) may also increase the yield, production, and/or efficiency of production of one or more fine chemicals from C. glutamicum cells containing such mutations. These pathogenesis proteins may be of utility for the survival of C. glutamicum cells in their natural environments. However, in the artificial environment of fermentor culture, nutrients are typically supplied in excess, and there should be no other organisms with which these bacteria need to compete. Thus, the synthesis of these pathogenesis proteins represents the utilization of energy and biomaterials which could instead be shifted to the production of one or more desired fine chemicals. Thus, by decreasing the number of such pathogenesis genes in C. glutamicum, it may be possible to increase the available intermediate compounds (e.g., nucleotides, amino acids, or energy molecules such as ATP) such that metabolism in general, and fine chemical production in particular is increased.

These RRP proteins involved in pathogenesis may themselves also be considered desirable fine chemicals. These proteins may have significant pharmaceutical applications, as, for example, antimicrobial or antifungal agents. Further, although C. glutamicum is not a human pathogen, its pathogenesis proteins (e.g., hemolysins or adhesins) may be similar in structure and activity to those from bacterial species which are significant human pathogens (e.g., E. coli or Pseudomonas spp.) These C. glutamicum pathogenesis proteins may thus serve as useful targets for the development of vaccines or therapeutics against various human pathogens. By mutagenizing the genes encoding these proteins such that their synthesis and/or translation is no longer repressed by cellular regulatory mechanisms, or such their production is no longer subject to feedback inhibition (e.g., by mutagenizing regulatory regions upstream or downstream of the gene, or by mutagenizing regulatory regions on the protein itself) greater numbers of these proteins may be able to be expressed and harvested from culture.

The aforementioned mutagenesis strategies for RRP proteins to result in increased yields of a fine chemical from C. glutamicum are not meant to be limiting; variations on these strategies will be readily apparent to one of ordinary skill in the art. Using such strategies, and incorporating the mechanisms disclosed herein, the nucleic acid and protein molecules of the invention may be utilized to generate C. glutamicum or related strains of bacteria expressing mutated RRP nucleic acid and protein molecules such that the yield, production, and/or efficiency of production of a desired compound is improved. This desired compound may be any product produced by C. glutamicum, which includes the final products of biosynthesis pathways and intermediates of naturally-occurring metabolic pathways, as well as molecules which do not naturally occur in the metabolism of C. glutamicum, but which are produced by a C. glutamicum strain of the invention.

This invention is further illustrated by the following examples which should not be construed as limiting. The contents of all references, patent applications, patents, published patent applications, Tables, Appendices, and the sequence listing cited throughout this application are hereby incorporated by reference.

Exemplification

### EXAMPLE 1

### Preparation of Total Genomic DNA of Corynebacterium Glutamicum ATCC 13032

A culture of Corynebacterium glutamicum (ATCC 13032) was grown overnight at 30° C. with vigorous shaking in BHI medium (Difco). The cells were harvested by centrifugation, the supernatant was discarded and the cells were resuspended in 5 ml buffer-I (5% of the original volume of the culture—all indicated volumes have been calculated for 100 ml of culture volume). Composition of buffer-I: 140.34 g/l sucrose, 2.46 g/l MgSO₄×7H₂O, 10 ml/I KH₂PO₄ solution (100 g/l, adjusted to pH 6.7 with KOH), 50 ml/I M12 concentrate (10 g/l (NH₄)₂SO₄, 1 g/l NaCl, 2 g/l MgSO_{4×7}H₂O, 0.2 g/l CaCl₂, 0.5 g/l yeast extract (Difco), 10 ml/l trace-elements-mix (200 mg/l FeSO₄×H₂O, 10 mg/l ZnSO₄×7 H₂O, 3 mg/l MnCl₂×4 H₂O, 30 mg/l H₃BO₃, 20 mg/l CoCl₂×6 H₂O, 1 mg/l NiCl₂×6 H₂O, 3 mg/l Na₂MoO_{4×2} H₂O, 500 mg/l complexing agent (EDTA or critic acid), 100 ml/l vitamins-mix (0.2 mg/l biotin, 0.2 mg/l folic acid, 20 mg/l p-amino benzoic acid, 20 mg/l riboflavin, 40 mg/l ca-panthothenate, 140 mg/l nicotinic acid, 40 mg/l pyridoxole hydrochloride, 200 mg/l myo-inositol). Lysozyme was added to the suspension to a final concentration of 2.5 mg/ml. After an approximately 4 h incubation at 37° C, the cell wall was degraded and the resulting protoplasts are harvested by centrifugation. The pellet was washed once with 5 ml buffer-I and once with 5 ml TE-buffer (10 mM Tris-HCl, 1 mM EDTA, pH 8). The pellet was resuspended in 4 ml TE-buffer and 0.5 ml SDS solution (10%) and 0.5 ml NaCl solution (5 M) are added. After adding of proteinase K to a final concentration of 200 μg/ml, the suspension is incubated for ca.18 h at 37° C. The DNA was purified by extraction with phenol, phenol-chloroform-isoamylalcohol and chloroform-isoamylalcohol using standard procedures. Then, the DNA was precipitated by adding 1/50 volume of 3 M sodium acetate and 2 volumes of ethanol, followed by a 30 min incubation at −20° C. and a 30 min centrifugation at 12,000 rpm in a high speed centrifuge using a SS34 rotor (Sorvall). The DNA was dissolved in 1 ml TE-buffer containing 20 μg/ml RNaseA and dialysed at 4° C. against 1000 ml TE-buffer for at least 3 hours. During this time, the buffer was exchanged 3 times. To aliquots of 0.4 ml of the dialysed DNA solution, 0.4 ml of 2 M LiCl and 0.8 ml of ethanol are added. After a 30 min incubation at −20° C., the DNA was collected by centriftigation (13,000 rpm, Biofuge Fresco, Heraeus, Hanau, Germany). The DNA pellet was dissolved in TE-buffer. DNA prepared by this procedure could be used for all purposes, including southern blotting or construction of genomic libraries.

### EXAMPLE 2

### Construction of Genomic Libraries in Escherichia Coli of Corynebacterium Glutamicum ATCC13032

Using DNA prepared as described in Example 1, cosmid and plasmid libraries were constructed according to known and well established methods (see e.g., Sambrook, J. et al. (1989) “Molecular Cloning: A Laboratory Manual”, Cold Spring Harbor Laboratory Press, or Ausubel, F. M. et al. (1994) “Current Protocols in Molecular Biology”, John Wiley & Sons.)

Any plasmid or cosmid could be used. Of particular use were the plasmids pBR322 (Sutcliffe, J. G. (1979) Proc. Natl. Acad. Sci. USA, 75:3737-3741); pACYC177 (Change & Cohen (1978) J. Bacteriol 134:1141-1156), plasmids of the pBS series (pBSSK+, pBSSK− and others; Stratagene, LaJolla, USA), or cosmids as SuperCos1 (Stratagene, LaJolla, USA) or Lorist6 (Gibson, T. J., Rosenthal A. and Waterson, R. H. (1987) Gene 53:283-286. Gene libraries specifically for use in C. glutamicum may be constructed using plasmid pSL109 (Lee, H.-S. and A. J. Sinskey (1994) J. Microbiol. Biotechnol. 4: 256-263).

### EXAMPLE 3

### DNA Sequencing and Computational Functional Analysis

Genomic libraries as described in Example 2 were used for DNA sequencing according to standard methods, in particular by the chain termination method using ABI377 sequencing machines (see e.g., Fleischman, R. D. et al. (1995) “Whole-genome Random Sequencing and Assembly of Haemophilus Influenzae Rd., Science, 269:496-512). Sequencing primers with the following nucleotide sequences were used: 5′-GGAAACAGTATGACCATG-3′ or 5′-GTAAAACGACGGCCAGT-3′.

### EXAMPLE 4

### In Vivo Mutagenesis

In vivo mutagenesis of Corynebacterium glutamicum can be performed by passage of plasmid (or other vector) DNA through E. coli or other microorganisms (e.g. Bacillus spp. or yeasts such as Saccharomyces cerevisiae) which are impaired in their capabilities to maintain the integrity of their genetic information. Typical mutator strains have mutations in the genes for the DNA repair system (e.g., mutHLS, mutD, mutT, etc.; for reference, see Rupp, W. D. (1996) DNA repair mechanisms, in: Escherichia coli and Salmonella, p. 2277-2294, ASM:

Washington.) Such strains are well known to one of ordinary skill in the art. The use of such strains is illustrated, for example, in Greener, A. and Callahan, M. (1994) Strategies 7: 32-34.

### EXAMPLE 5

### DNA Transfer Between Escherichia Coli and Corynebacterium Glutamicum

Several Corynebacterium and Brevibacterium species contain endogenous plasmids (as e.g., pHM1519 or pBL1) which replicate autonomously (for review see, e.g., Martin, J. F. et al. (1987) Biotechnology, 5:137-146). Shuttle vectors for Escherichia coli and Corynebacterium glutamicum can be readily constructed by using standard vectors for E. coli (Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons) to which a origin or replication for and a suitable marker from Corynebacterium glutamicum is added. Such origins of replication are preferably taken from endogenous plasmids isolated from Corynebacterium and Brevibacterium species. Of particular use as transformation markers for these species are genes for kanamycin resistance (such as those derived from the Tn5 or Tn903 transposons) or chloramphenicol (Winnacker, E. L. (1987) “From Genes to Clones—Introduction to Gene Technology, VCH, Weinheim). There are numerous examples in the literature of the construction of a wide Variety of shuttle vectors which replicate in both E. coli and C. glutamicum, and which can be used for several purposes, including gene over-expression (for reference, see e.g., Yoshihama, M. et al. (1985) J. Bacteriol. 162:591-597, Martin J. F. et al. (1987) Biotechnology, 5:137-146 and Eikmanns, B. J. et al. (1991) Gene, 102:93-98).

Using standard methods, it is possible to clone a gene of interest into one of the shuttle vectors described above and to introduce such a hybrid vectors into strains of Corynebacterium glutamicum. Transformation of C. glutamicum can be achieved by protoplast transformation (Kastsumata, R. et al. (1984) J. Bacteriol. 159306-311), electroporation (Liebl, E. et al. (1989) FEMS Microbiol. Letters, 53:399-303) and in cases where special vectors are used, also by conjugation (as described e.g. in Schäfer, A et al. (1990) J. Bacteriol. 172:1663-1666). It is also possible to transfer the shuttle vectors for C. glutamicum to E. coli by preparing plasmid DNA from C. glutamicum (using standard methods well-known in the art) and transforming it into E. coli. This transformation step can be performed using standard methods, but it is advantageous to use an Mcr-deficient E. coli strain, such as NM522 (Gough & Murray (1983) J. Mol. Biol. 166:1-19).

Genes may be overexpressed in C. glutamicum strains using plasmids which comprise pCGI (U.S. Pat. No. 4,617,267) or fragments thereof, and optionally the gene for kanamycin resistance from TN903 (Grindley, N. D. and Joyce, C. M. (1980) Proc. Natl. Acad. Sci. USA 77(12): 7176-7180). In addition, genes may be overexpressed in C. glutamicum strains using plasmid pSL109 (Lee, H.-S. and A. J. Sinskey (1994) J. Microbiol. Biotechnol. 4: 256-263).

Aside from the use of replicative plasmids, gene overexpression can also be achieved by integration into the genome. Genomic integration in C. glutamicum or other Corynebacterium or Brevibacterium species may be accomplished by well-known methods, such as homologous recombination with genomic region(s), restriction endonuclease mediated integration (REMI) (see, e.g., DE Patent 19823834), or through the use of transposons. It is also possible to modulate the activity of a gene of interest by modifying the regulatory regions (e.g., a promoter, a repressor, and/or an enhancer) by sequence modification, insertion, or deletion using site-directed methods (such as homologous recombination) or methods based on random events (such as transposon mutagenesis or REMI). Nucleic acid sequences which function as transcriptional terminators may also be inserted 3′, to the coding region of one or more genes of the invention; such terminators are well-known in the art and are described, for example, in Winnacker, E. L. (1987) From Genes to Clones—Introduction to Gene Technology. VCH: Weinheim.

### EXAMPLE 6

### Assessment of the Expression of the Mutant Protein

Observations of the activity of a mutated protein in a transformed host cell rely on the fact that the mutant protein is expressed in a similar fashion and in a similar quantity to that of the wild-type protein. A useful method to ascertain the level of transcription of the mutant gene (an indicator of the amount of mRNA available for translation to the gene product) is to perform a Northern blot (for reference see, for example, Ausubel et al. (1988) Current Protocols in Molecular Biology, Wiley: N.Y.), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of mRNA for this gene. This information is evidence of the degree of transcription of the mutant gene. Total cellular RNA can be prepared from Corynebacterium glutamicum by several methods, all well-known in the art, such as that described in Bormann, E. R. et al. (1992) Mol. Microbiol. 6: 317-326.

To assess the presence or relative quantity of protein translated from this mRNA, standard techniques, such as a Western blot, may be employed (see, for example, Ausubel et al. (1988) Current Protocols in Molecular Biology, Wiley: N.Y.). In this process, total cellular proteins are extracted, separated by gel electrophoresis, transferred to a matrix such as nitrocellulose, and incubated with a probe, such as an antibody,-which specifically binds to the desired protein. This probe is generally tagged with a chemiluminescent or calorimetric label which may be readily detected. The presence and quantity of label observed indicates the presence and quantity of the desired mutant protein present in the cell.

### EXAMPLE 7

### Growth of Genetically Modified Corynebacterium glutamicum—Media and Culture Conditions

Genetically modified Corynebacteria are cultured in synthetic or natural growth media. A number of different growth media for Corynebacteria are both well-known and readily available (Lieb et al. (1989) Appl. Microbiol. Biotechnol., 32:205-210; von der Osten et al. (1998) Biotechnology Letters, 11:11-16; Patent DE 4,120;867; Liebl (1992) “The Genus Corynebacteriumi in: The Procaryotes, Volume II, Balows, A. et al., eds. Springer-Verlag). These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribulose, lactose, maltose, sucrose, raffinose, starch or cellulose serve as very good carbon sources. It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂SO₄, NH₄OH, nitrates, urea, amino acids or complex nitrogen sources like corn steep liquor, soy bean flour, soy bean protein, yeast extract, meat extract and others.

Inorganic salt compounds which may be included in the media include the chloride-, phosphorous- or sulfate-salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamin, folic acid, nicotinic acid, pantothenate and pyridoxin. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (eds. P. M. Rhodes, P. F. Stanbury, IRL Press (1997) pp. 53-73, ISBN 0 19 963577 3). It is also possible to select growth media from commercial suppliers, like standard 1 (Merck) or BHI (grain heart infusion, DIFCO) or others.

All medium components are sterilized, either by heat (20 minutes at 1.5 bar and 121° C.) or by sterile filtration. The components can either be sterilized together or, if necessary, separately. All media components can be present at the beginning of growth, or they can optionally be added continuously or batchwise.

Culture conditions are defined separately for each experiment. The temperature should be in a range between 15° C. and 45° C. The temperature can be kept constant or can be altered during the experiment. The pH of the medium should be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the micro-organisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml shake flasks are used, filled with 10% (by volume) of the required growth medium. The flasks should be shaken on a rotary shaker (amplitude 25 mm) using a speed-range of 100-300 rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD₆₀₀ of 0.5-1.5 using cells grown on agar plates, such as CM plates (10 g/l glucose, 2,5 g/l NaCl, 2 g/l urea, 10 g/l polypeptone, 5 g/l yeast extract, 5 g/l meat extract, 22 g/l NaCl, 2 g/l urea, 10 g/l polypeptone, 5 g/l yeast extract, 5 g/l meat extract, 22 g/l agar, pH 6.8 with 2M NaOH) that had been incubated at 30° C. Inoculation of the media is accomplished by either introduction of a saline suspension of C. glutamicum cells from CM plates or addition of a liquid preculture of this bacterium.

### EXAMPLE 8

### In Vitro Analysis of the Function of Mutant Proteins

The determination of activities and kinetic parameters of enzymes is well established in the art. Experiments to determine the activity of any given altered enzyme must be tailored to the specific activity of the wild-type enzyme, which is well within the ability of one of ordinary skill in the art. Overviews about enzymes in general, as well as specific details concerning structure, kinetics, principles, methods, applications and examples for the determination of many enzyme activities may be found, for example, in the following references: Dixon, M., and Webb, E. C., (1979) Enzymes. Longrnans: London; Fersht, (1985) Enzyme Structure and Mechanism. Freeman: N.Y.; Walsh, (1979) Enzymatic Reaction Mechanisms. Freeman: San Francisco; Price, N. C., Stevens, L. (1982) Fundamentals of Enzymology. Oxford Univ. Press: Oxford; Boyer, P. D., ed. (1983) The Enzymes, 3^{rd} ed. Academic Press: New York; Bisswanger, H., (1994) Enzymkinetik, 2^{nd} ed. VCH: Weinheim (ISBN 3527300325); Bergmeyer, H. U., Bergmeyer; J., Graβ1, M., eds. (1983-1986) Methods of Enzymatic Analysis, 3^{rd} ed., vol. I-XII, Verlag Chemie: Weinheim; and Ullmann's Encyclopedia of Industrial Chemistry (1987) vol. A9, “Enzymes”. VCH: Weinheim, p. 352-363.

The activity of proteins which bind to DNA can be measured by several well-established methods, such as DNA band-shift assays (also called gel retardation assays). The effect of such proteins on the expression of other molecules can be measured using reporter gene assays (such as that described in Kolmar, H. et al. (1995) EMBO J. 14: 3895-3904 and references cited therein). Reporter gene test systems are well known and established for applications in both pro- and eukaryotic cells, using enzymes such as beta-galactosidase, green fluorescent protein, and several others.

The determination of activity of membrane-transport proteins can be performed according to techniques such as those described in Gennis, R. B. (1989) “Pores, Channels and Transporters”, in Biomembranes, Molecular Structure and Function, Springer: Heidelberg, p. 85-137; 199-234; and 270-322.

### EXAMPLE 9

### Analysis of Impact of Mutant Protein on the Production of the Desired Product

The effect of the genetic modification in C. glutamicum on production of a desired compound (such as an amino acid) can be assessed by growing the modified microorganism under suitable conditions (such as those described above) and analyzing the medium and/or the cellular component for increased production of the desired product (i.e., an amino acid). Such analysis techniques are well known to one of ordinary skill in the art, and include spectroscopy, thin layer chromatography, staining methods of various kinds, enzymatic and microbiological methods, and analytical chromatography such as high performance liquid chromatography (see, for example, Ullman, Encyclopedia of Industrial Chemistry, vol. A2, p. 89-90 and p. 443-613, VCH: Weinheim (1985); Fallon, A. et al., (1987) “Applications of HPLC in Biochemistry” in: Laboratory Techniques in Biochemistry and Molecular Biology, vol. 17; Rehm et al. (1993) Biotechnology, vol. 3, Chapter III: “Product recovery and purification”, page 469-714, VCH: Weinheim; Belter, P. A. et al. (1988) Bioseparations: downstream processing for biotechnology, John Wiley and Sons; Kennedy, J. F. and Cabral, J. M. S. (1992) Recovery processes for biological materials, John Wiley and Sons; Shaeiwitz, J. A. and Henry, J. D. (1988) Biochemical separations, in: Ulmann's Encyclopedia of Industrial Chemistry, vol. B3, Chapter 11, page 1-27, VCH: Weinheim; and Dechow, F. J. (1989) Separation and purification techniques in biotechnology, Noyes Publications.)

In addition to the measurement of the final product of fermentation, it is also possible to analyze other components of the:metabolic pathways utilized for the production of the desired compound, such as intermediates and side-products, to determine the overall efficiency of production of the compound. Analysis methods include measurements of nutrient levels in the medium (e.g., sugars, hydrocarbons, nitrogen sources, phosphate, and other ions), measurements of biomass composition and growth, analysis of the production of common metabolites of biosynthetic pathways, and measurement of gasses produced during fermentation. Standard methods for these measurements are outlined in Applied Microbial Physiology, A Practical Approach, P. M. Rhodes and P. F. Stanbury, eds., IRL Press, p. 103-129; 131-163; and 165-192 (ISBN: 0199635773) and references cited therein.

### EXAMPLE 10

### Purification of the Desired Product from C glutamicum Culture

Recovery of the desired product from the C. glutamicum cells or supernatant of the above-described culture can be performed by various methods well known in the art. If the desired product is not secreted from the cells, the cells can be harvested from the culture by low-speed centrifugation, the cells can be lysed by standard techniques, such as mechanical force or sonication. The cellular debris is removed by centrifugation, and the supernatant fraction containing the soluble proteins is retained for further purification of the desired compound. If the product is secreted from the C. glutamicum cells, then the cells are removed from the culture by low-speed centrifugation, and the supernate fraction is retained for further purification.

The supernatant fraction from either purification method is subjected to chromatography with a suitable resin, in which the desired molecule is either retained on a chromatography resin while many of the impurities in the sample are not, or where the impurities are retained by the resin while the sample is not. Such chromatography steps may be repeated as necessary, using the same or different chromatography resins. One of ordinary skill in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There are a wide array of purification methods known to the art and the preceding method of purification is not meant to be limiting. Such purification techniques are described, for example, in Bailey, J. E. & Ollis, D. F. Biochemical Engineering Fundamentals, McGraw-Hill: N.Y. (1986).

The identity and purity of the isolated compounds may be assessed by techniques standard in the art. These include high-performance liquid chromatography (HPLC), spectroscopic methods, staining methods, thin layer chromatography, NIRS, enzymatic assay, or microbiologically. Such analysis methods are reviewed in: Patek et al. (1994) Appl. Environ. Microbiol. 60:133-140; Malakhova et al.; (1996) Biotekhnologiya 11: 27-32; and Schmidt et al. (1998) Bioprocess Engineer. 19:67-70. Ulmann's Encyclopedia of Industrial Chemistry, (1996) vol. A27, VCH: Weinheim, p. 89-90, p. 521-540, p. 540-547) p. 559-566, 575-581 and p. 581-587; Michal, G. (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, vol. 17.

### EXAMPLE 11

### Analysis of the Gene Sequences of the Invention

The comparison of sequences and determination of percent homology between two sequences are art-known techniques, and can be accomplished using a mathematical algorithm, such as the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to RRP nucleic acid molecules of the invention. BLAST protein searches can be performed with the XBLAST program, score=50, wordlength=3 to obtain amino acid sequences homologous to RRP protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, one of ordinary skill in the art will know how to optimize the parameters of the program (e.g., XBLAST and NBLAST) for the specific sequence being analyzed.

Another example of a mathematical algorithm utilized for the comparison of sequences is the algorithm of Meyers and Miller ((1988) Comput. Appl. Biosci. 4: 11-17). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art, and include ADVANCE and ADAM. described in Torelli and Robotti (1994) Comput. Appl. Biosci. 10:3-5; and FASTA, described in Pearson and Lipman (1988) P.N.A.S. 85:2444-8.

The percent homology between two amino acid sequences can also be accomplished using the GAP program in the GCG software package (available at http://www.gcg.com), using either a Blosum 62 matrix or a PAM250 matrix, and a gap weight of 12, 10, 8, 6, or 4 and a length weight of 2, 3, or 4. The percent homology between two nucleic acid sequences can be accomplished using the GAP program in the GCG software package, using standard parameters, such as a gap weight of 50 and a length weight of 3.

A comparative analysis of the gene sequences of the invention with those present in Genbank has been performed using techniques known in the art (see, e.g., Bexevanis and Ouellette, eds. (1998) Bioinformatics: A Practical Guide to the Analysis of Genes and Proteins. John Wiley and Sons: New York). The gene sequences of the invention were compared to genes present in Genbank in a three-step process. In a first step, a BLASTN analysis (e.g., a local alignment analysis) was performed for each of the sequences of the invention against the nucleotide sequences present in Genbank, and the top 500 hits were retained for further analysis. A subsequent FASTA search (e.g., a combined local and global alignment analysis, in which limited regions of the sequences are aligned) was performed on these 500 hits. Each gene sequence of the invention was subsequently globally aligned to each of the top three FASTA hits, using the GAP program in the GCG software package (using standard parameters). In order to obtain correct results, the length of the sequences extracted from Genbank were adjusted to the length of the query sequences by methods well-known in the art. The results of this analysis are set forth in Table 4. The resulting data is identical to that which would have been obtained had a GAP (global) analysis alone been performed on each of the genes of the invention in comparison with each of the references in Genbank, but required significantly reduced computational time as compared to such a database-wide GAP (global) analysis. Sequences of the invention for which no alignments above the cutoff values were obtained are indicated on Table 4 by the absence of alignment information. It will further be understood by one of ordinary skill in the art that the GAP alignment homology percentages set forth in Table 4 under the heading “% homology (GAP)” are listed in the European numerical format, wherein a ‘,’ represents a decimal point. For example, a value of “40,345” in this column represents “40.345%”.

### EXAMPLE 12

### Construction and Operation of DNA Microarrays

The sequences of the invention may additionally be used in the construction and application of DNA microarrays (the design, methodology, and uses of DNA arrays are well known in the art, and are described, for example, in Schena, M. et al. (1995) Science 270: 467-470; Wodicka, L. et al. (1997) Nature Biotechnology 15: 1359-1367; DeSaizieu, A. et al. (1998) Nature Biotechnology 16: 45-48; and DeRisi, J. L. et al. (1997) Science 278: 680-686).

DNA microarrays are solid or flexible supports consisting of nitrocellulose, nylon, glass, silicone, or other materials. Nucleic acid molecules may be attached to the surface in an ordered manner. After appropriate labeling, other nucleic acids or nucleic acid mixtures can be hybridized to the immobilized nucleic acid molecules, and the label may be used to monitor and measure the individual signal intensities of the hybridized molecules at defined regions. This methodology allows the simultaneous quantification of the relative or absolute amount of all or selected nucleic acids in the applied nucleic acid sample or mixture. DNA microarrays; therefore, permit an analysis of the expression of multiple (as many as 6800 or more) nucleic acids in parallel (see, e.g., Schena, M. (1996) BioEssays 18(5): 427-431).

The sequences of the invention may be used to design oligonucleotide primers which are able to amplify defined regions of one or more C. glutamicum genes by a nucleic acid amplification reaction such as the polymerase chain reaction. The choice and design of the 5′ or 3′ oligonucleotide primers or of appropriate linkers allows the covalent attachment of the resulting PCR products to the surface of a support medium described above (and also described, for example, Schena, M. et al. (1995) Science 270: 467-470).

Nucleic acid microarrays may also be constructed by in situ oligonucleotide synthesis as described by Wodicka, L. et al. (1997) Nature Biotechnology 15: 1359-1367. By photolithographic methods, precisely defined regions of the matrix are exposed to light. Protective groups which are photolabile are thereby activated and undergo nucleotide addition, whereas regions that are masked from light do not undergo any modification. Subsequent cycles of protection and light activation permit the synthesis of different oligonucleotides at defined positions. Small, defined regions of the genes of the invention may be synthesized on microarrays by solid phase oligonucleotide synthesis.

The nucleic acid molecules of the invention present in a sample or mixture of nucleotides may be hybridized to the microarrays. These nucleic acid molecules can be labeled according to standard methods. In brief, nucleic acid molecules (e.g., mRNA molecules or DNA molecules) are labeled by the incorporation of isotopically or fluorescently labeled nucleotides, e.g., during reverse transcription or DNA synthesis. Hybridization of labeled nucleic acids to microarrays is described (e.g., in Schena, M. et al. (1995) supra; Wodicka, L. et al. (1997), supra; and DeSaizieu A. et al. (1998), supra). The detection and quantification of the hybridized molecule are tailored to the specific incorporated label. Radioactive labels can be detected, for example, as described in Schena, M. et al. (1995) supra) and fluorescent labels may be detected, for example, by the method of Shalon et al. (1996) Genome Research 6: 639-645).

The application of the sequences of the invention to DNA microarray technology, as described above, permits comparative analyses of different strains of C. glutamicum or other Corynebacteria. For example, studies of inter-strain variations based on individual transcript profiles and the identification of genes that are important for specific and/or desired strain properties such as pathogenicity, productivity and stress tolerance are facilitated by nucleic acid array methodologies. Also, comparisons of the profile of expression of genes of the invention during the course of a fermentation reaction are possible using nucleic acid array technology.

### EXAMPLE 13

### Analysis of the Dynamics of Cellular Protein Populations (Proteomics)

The genes, compositions, and methods of the invention may be applied to study the interactions and dynamics of populations of proteins, termed ‘proteomics’. Protein populations of interest include, but are not limited to, the total protein population of C. glutamicum (e.g., in comparison with the protein populations of other organisms), those proteins which are active under specific environmental or metabolic conditions (e.g., during fermentation, at high or low temperature, or at high or low pH), or those proteins which are active during specific phases of growth and development.

Protein populations can be analyzed by various well-known techniques, such as gel electrophoresis. Cellular proteins may be obtained, for example, by lysis or extraction, and may be separated from one another using a variety of electrophoretic techniques. Sodium dodecyl sulfate polyacrylamide gel electrophoresis (SDS-PAGE) separates proteins largely on the basis of their molecular weight. Isoelectric focusing polyacrylamide gel electrophoresis (IEF-PAGE) separates proteins by their isoelectric point (which reflects not only the amino acid sequence but also posttranslational modifications of the protein). Another, more preferred method of protein analysis is the consecutive combination of both IEF-PAGE and SDS-PAGE, known as 2-D-gel electrophoresis (described, for example, in Hermann et al. (1998) Electrophoresis 19: 3217-3221; Fountoulakis et al. (1998) Electrophoresis 19: 1193-1202; Langen et al. (1997) Electrophoresis 18: 1184-1192; Antelmann et al. (1997) Electrophoresis 18: 1451-1463). Other separation techniques may also be utilized for protein separation, such as capillary gel electrophoresis; such techniques are well known in the art.

Proteins separated by these methodologies can be visualized by standard techniques, such as by staining or labeling. Suitable stains are known in the art, and include Coomassie Brilliant Blue, silver stain, or fluorescent dyes such as Sypro Ruby (Molecular Probes). The inclusion of radioactively labeled amino acids or other protein precursors (e.g., ³⁵S-methionine, ³⁵S-cysteine, ¹⁴C-labelled amino acids, ¹⁵N-amino acids, ¹⁵NO₃ or ¹⁵NH₄⁺ or ¹³C-labelled amino acids) in the medium of C. glutamicum permits the labeling of proteins from these cells prior to their separation. Similarly, fluorescent labels may be employed. These labeled proteins can be extracted, isolated and separated according to the previously described techniques.

Proteins visualized by these techniques can be further analyzed by measuring the amount of dye or label used. The amount of a given protein can be determined quantitatively using, for example, optical methods and can be compared to the amount of other proteins in the same gel or in other gels. Comparisons of proteins on gels can be made, for example, by optical comparison, by spectroscopy, by image scanning and analysis of gels, or through the use of photographic films and screens. Such techniques are well-known in the art.

To determine the identity of any given protein, direct sequencing or other standard techniques may be employed. For example, N— and/or C-terminal amino acid sequencing (such as Edman degradation) may be used, as may mass spectrometry (in particular MALDI or ESI techniques (see, e.g., Langen et al. (1997) Electrophoresis 18: 1184-1192)). The protein sequences provided herein can be used for the identification of C. glutamicum proteins by these techniques.

The information obtained by these methods can be used to compare patterns of protein presence, activity, or modification between different samples from various biological conditions (e.g., different organisms, time points of fermentation, media conditions, or different biotopes, among others). Data obtained from such experiments alone, or in combination with other techniques, can be used for various applications, such as to compare the behavior of various organisms in a given (e.g., metabolic) situation, to increase the productivity of strains which produce fine chemicals or to increase the efficiency of the production of fine chemicals.

Equivalents

Those skilled of ordinary skill in the art will recognize, or will be able to ascertain using no more than routine experimentation, many equivalents to the specific embodiments of the invention described herein. Such equivalents are intended to be encompassed by the following claims.

TABLE 1
Genes in the Application

| Nucleic | Amino | | | | | |
|---|---|---|---|---|---|---|
| Acid | Acid | | | | | |
| SEQ | SEQ | | | | | |
| ID | ID | Identification | | | | |
| NO | NO | Code | Contig. | NT Start | NT Stop | Function |
| 1 | 2 | RXN00625 | VV0135 | 5 | 946 | (D90902) extracellular nuclease [Synechocystis sp.] |
| 3 | 4 | F RXA00625 | GR00163 | 9320 | 8982 | (D90902) extracellular nuclease [Synechocystis sp.] |
| 5 | 6 | RXN02943 | VV0103 | 1671 | 4 | (D90902) extracellular nuclease [Synechocystis sp.] |
| 7 | 8 | F RXA00538 | GR00139 | 1272 | 4 | (D90902) extracellular nuclease [Synechocystis sp.] |
| 9 | 10 | RXA00823 | GR00221 | 3566 | 4345 | ENDONUCLEASE III (EC 4.2.99.18) |
| 11 | 12 | RXA02145 | GR00639 | 12248 | 13864 | ENDONUCLEASE III (EC 4.2.99.18) |
| 13 | 14 | RXA01064 | GR00297 | 937 | 1572 | THERMONUCLEASE PRECURSOR (EC 3.1.31.1) |
| 15 | 16 | RXN01594 | VV0229 | 12195 | 11377 | HEMOLYSIN |
| 17 | 18 | F RXA01594 | GR00447 | 2580 | 3323 | HEMOLYSIN |
| 19 | 20 | RXA01718 | GR00488 | 540 | 55 | HEMOLYSIN |
| 21 | 22 | RXN03148 | VV0146 | 626 | 991 | HEMOLYSIN |
| 23 | 24 | RXN00562 | VV0103 | 5761 | 6483 | HEMOLYSIN III |
| 25 | 26 | F RXA00562 | GR00150 | 405 | 1034 | HEMOLYSIN III |
| 27 | 28 | RXN00890 | VV0099 | 18771 | 20069 | HEMOLYSIN |
| 29 | 30 | F RXA00890 | GR00242 | 15953 | 17227 | HEMOLYSIN |

Genes involved in DNA replication, topology, and packaging

| | | | | | | |
|---|---|---|---|---|---|---|
| 31 | 32 | RXN01772 | VV0050 | 28644 | 33581 | /K/J Superfamily II DNA and RNA helicases |
| 33 | 34 | F RXA01772 | GR00502 | 4731 | 2368 | /K/J Superfamily II DNA and RNA helicases |
| 35 | 36 | RXA02363 | GR00685 | 10755 | 15554 | /K/J Superfamily II DNA and RNA helicases |
| 37 | 38 | RXN01606 | VV0137 | 5576 | 2901 | Hypothetical ATP-Dependent RNA Helicase |
| 39 | 40 | F RXA01797 | GR00508 | 1249 | 542 | /L Superfamily II DNA and RNA(?) helicases (SNF2 family) |
| 41 | 42 | RXN01030 | VV0015 | 32429 | 33604 | Hypothetical ATP-dependent RNA helicase |
| 43 | 44 | F RXA01030 | GR00295 | 2007 | 3182 | /L Superfamily II DNA and RNA(?) helicases (SNF2 family) |
| 45 | 46 | RXA01739 | GR00493 | 4702 | 5298 | Superfamily I DNA and RNA helicases |
| 47 | 48 | RXA02359 | GR00685 | 1666 | 3534 | Superfamily I DNA and RNA helicases |
| 49 | 50 | RXN02764 | VV0317 | 2787 | 4 | DNA HELICASE II (EC 3.6.1.—) |
| 51 | 52 | F RXA02764 | GR00769 | 2787 | 4 | Superfamily I DNA and RNA helicases |
| 53 | 54 | RXA01736 | GR00493 | 3 | 2870 | (AL021646) putative ATP-dependent DNA helicase |
| | | | | | | [Mycobacterium tuberculosis] |
| 55 | 56 | RXA00095 | GR00014 | 4677 | 2389 | DNA HELICASE II (EC 3.6.1.—) |
| 57 | 58 | RXN02819 | VV0088 | 6162 | 5116 | ATP-DEPENDENT DNA HELICASE |
| 59 | 60 | F RXA02819 | GR00800 | 615 | 4 | ATP-DEPENDENT DNA HELICASE |
| 61 | 62 | RXA01157 | GR00328 | 1610 | 6 | ATP-DEPENDENT DNA HELICASE |
| 63 | 64 | RXN01876 | VV0145 | 188 | 2038 | ATP-DEPENDENT DNA HELICASE REP (EC 3.6.1.—) |
| 65 | 66 | F RXA01876 | GR00536 | 2796 | 1324 | ATP-DEPENDENT DNA HELICASE REP (EC 3.6.1.—) |
| 67 | 68 | RXA00544 | GR00140 | 1639 | 3168 | REPLICATIVE DNA HELICASE (EC 3.6.1.—) |
| 69 | 70 | RXA01866 | GR00533 | 326 | 6 | ATP-DEPENDENT HELICASE HRPA |
| 71 | 72 | RXA01867 | GR00533 | 853 | 362 | ATP-DEPENDENT HELICASE HRPA |
| 73 | 74 | RXN03166 | VV0322 | 2933 | 699 | ATP-DEPENDENT HELICASE HRPA |
| 75 | 76 | F RXA00361 | GR00072 | 1853 | 6 | ATP-DEPENDENT HELICASE HRPA |
| 77 | 78 | RXN02293 | VV0127 | 19979 | 22366 | ATP-DEPENDENT HELICASE HRPB |
| 79 | 80 | F RXA02293 | GR00662 | 2819 | 5206 | ATP-dependent helicase |
| 81 | 82 | RXA02755 | GR00766 | 951 | 2945 | PROBABLE ATP-DEPENDENT HELICASE DING |
| 83 | 84 | RXN01374 | VV0091 | 7624 | 8865 | Hypothetical ATP-Dependent RNA Helicase |
| 85 | 86 | F RXA01374 | GR00400 | 885 | 4 | PROBABLE ATP-DEPENDENT HELICASE HEPA |
| 87 | 88 | RXN00817 | VV0054 | 35789 | 33396 | PROBABLE ATP-DEPENDENT HELICASE LHR (EC 3.6.1.—) |
| 89 | 90 | F RXA00809 | GR00218 | 514 | 5 | PROBABLE ATP-DEPENDENT HELICASE LHR (EC 3.6.1.—) |
| 91 | 92 | F RXA00817 | GR00219 | 6388 | 4679 | PROBABLE ATP-DEPENDENT HELICASE LHR (EC 3.6.1.—) |
| 93 | 94 | RXN00103 | VV0129 | 11188 | 15747 | PROBABLE ATP-DEPENDENT HELICASE LHR (EC 3.6.1.—) |
| 95 | 96 | F RXA00103 | GR00014 | 15860 | 11301 | PROBABLE ATP-DEPENDENT HELICASE LHR (EC 3.6.1.—) |
| 97 | 98 | RXN02357 | VV0051 | 23376 | 17077 | PROBABLE ATP-DEPENDENT HELICASE LHR (EC 3.6.1.—) |
| 99 | 100 | F RXA01363 | GR00395 | 1408 | 2106 | PROBABLE ATP-DEPENDENT HELICASE LHR (EC 3.6.1.—) |
| 101 | 102 | F RXA02357 | GR00685 | 3 | 1205 | PROBABLE ATP-DEPENDENT HELICASE LHR (EC 3.6.1.—) |
| 103 | 104 | F RXA02785 | GR00776 | 2 | 3856 | PROBABLE ATP-DEPENDENT HELICASE LHR (EC 3.6.1.—) |
| 105 | 106 | RXA01740 | GR00493 | 5314 | 6735 | PUTATIVE DNA HELICASE II HOMOLOG (EC 3.6.1.—) |
| 107 | 108 | RXN01683 | VV0179 | 5275 | 7842 | DNA GYRASE SUBUNIT A (EC 5.99.1.3) |
| 109 | 110 | F RXA01682 | GR00468 | 1 | 234 | DNA GYRASE SUBUNIT A (EC 5.99.1.3) |
| 111 | 112 | F RXA01683 | GR00468 | 146 | 895 | DNA GYRASE SUBUNIT A (EC 5.99.1.3) |
| 113 | 114 | F RXA01684 | GR00469 | 3 | 875 | DNA GYRASE SUBUNIT A (EC 5.99.1.3) |
| 115 | 116 | RXN01688 | VV0179 | 1 | 930 | DNA GYRASE SUBUNIT B (EC 5.99.1.3) |
| 117 | 118 | F RXA01688 | GR00471 | 1 | 564 | DNA GYRASE SUBUNIT B (EC 5.99.1.3) |
| 119 | 120 | RXN01689 | VV0221 | 1920 | 3035 | DNA GYRASE SUBUNIT B (EC 5.99.1.3) |
| 121 | 122 | F RXA01689 | GR00472 | 3 | 728 | DNA GYRASE SUBUNIT B (EC 5.99.1.3) |
| 123 | 124 | F RXA01735 | GR00492 | 1213 | 1494 | DNA GYRASE SUBUNIT B (EC 5.99.1.3) |
| 125 | 126 | RXN03093 | VV0054 | 36970 | 38808 | DNA TOPOISOMERASE I (EC 5.99.1.2) |
| 127 | 128 | F RXA00798 | GR00213 | 2525 | 171 | DNA TOPOISOMERASE I (EC 5.99.1.2) |
| 129 | 130 | RXN00990 | VV0210 | 4962 | 4498 | ATP-DEPENDENT RNA HELICASE DEAD |
| 131 | 132 | F RXA00990 | GR00281 | 2 | 454 | ATP-DEPENDENT RNA HELICASE DEAD |
| 133 | 134 | RXN00994 | VV0106 | 356 | 6 | ATP-DEPENDENT RNA HELICASE DEAD |
| 135 | 136 | F RXA00994 | GR00282 | 797 | 1138 | ATP-DEPENDENT RNA HELICASE DEAD |
| 137 | 138 | RXN02468 | VV0211 | 760 | 1983 | ATP-DEPENDENT RNA HELICASE DEAD |
| 139 | 140 | F RXA02463 | GR00713 | 141 | 254 | ATP-DEPENDENT RNA HELICASE DEAD |
| 141 | 142 | F RXA02468 | GR00714 | 760 | 1290 | ATP-DEPENDENT RNA HELICASE DEAD |
| 143 | 144 | RXA00050 | GR00008 | 5451 | 3256 | ATP-DEPENDENT RNA HELICASE DEAD |
| 145 | 146 | RXA02682 | GR00754 | 6902 | 6576 | DNA-BINDING PROTEIN |
| 147 | 148 | RXN00542 | VV0079 | 36158 | 36832 | SINGLE-STRAND BINDING PROTEIN |
| 149 | 150 | F RXA00542 | GR00140 | 1 | 519 | SINGLE-STRAND BINDING PROTEIN |
| 151 | 152 | RXN02833 | VV0050 | 823 | 41 | CHROMOSOMAL REPLICATION INITIATOR PROTEIN DNAA |
| 153 | 154 | F RXA02833 | GR00822 | 627 | 49 | CHROMOSOMAL REPLICATION INITIATOR PROTEIN DNAA |
| 155 | 156 | RXA01480 | GR00422 | 14550 | 12658 | DNA PRIMASE (EC 2.7.7.—) |
| 157 | 158 | RXN03163 | VV0204 | 4514 | 6010 | PRIMOSOMAL PROTEIN N′ |
| 159 | 160 | F RXA02241 | GR00654 | 8446 | 10473 | PRIMOSOMAL PROTEIN N′, replication factor Y |
| 161 | 162 | RXN00061 | VV0044 | 4256 | 1590 | DNA POLYMERASE I (EC 2.7.7.7) |
| 163 | 164 | F RXA00060 | GR00009 | 9187 | 11643 | DNA POLYMERASE I (EC 2.7.7.7) |
| 165 | 166 | F RXA00061 | GR00009 | 11643 | 11852 | DNA POLYMERASE I (EC 2.7.7.7) |
| 167 | 168 | RXA02657 | GR00752 | 11033 | 14614 | DNA POLYMERASE III, ALPHA CHAIN (EC 2.7.7.7) |
| 169 | 170 | RXA01238 | GR00358 | 2965 | 4365 | DNA POLYMERASE III, ALPHA CHAIN (EC 2.7.7.7) |
| 171 | 172 | RXN00407 | VV0086 | 55677 | 58340 | DNA POLYMERASE III, ALPHA CHAIN (EC 2.7.7.7) |
| 173 | 174 | F RXA00407 | GR00091 | 3 | 578 | DNA POLYMERASE III, ALPHA CHAIN (EC 2.7.7.7) |
| 175 | 176 | F RXA00415 | GR00092 | 4519 | 6480 | DNA POLYMERASE III, ALPHA CHAIN (EC 2.7.7.7) |
| 177 | 178 | RXN00414 | VV0086 | 55331 | 55684 | DNA POLYMERASE III, ALPHA CHAIN (EC 2.7.7.7) |
| 179 | 180 | F RXA00414 | GR00092 | 4172 | 4591 | DNA POLYMERASE III, ALPHA CHAIN (EC 2.7.7.7) |
| 181 | 182 | RXN00807 | VV0009 | 63097 | 61856 | DNA POLYMERASE III, DELTA′ SUBUNIT (EC 2.7.7.7) |
| 183 | 184 | F RXA00807 | GR00216 | 423 | 1541 | DNA POLYMERASE III, DELTA′ SUBUNIT (EC 2.7.7.7) |
| 185 | 186 | RXA00214 | GR00032 | 13046 | 13756 | DNA POLYMERASE III, EPSILON CHAIN (EC 2.7.7.7) |
| 187 | 188 | RXA01255 | GR00365 | 4919 | 3840 | DNA POLYMERASE III, EPSILON CHAIN (EC 2.7.7.7) |
| 189 | 190 | RXN00066 | VV0012 | 816 | 4 | DNA POLYMERASE III SUBUNITS GAMMA AND TAU (EC 2.7.7.7) |
| 191 | 192 | F RXA00066 | GR00010 | 4494 | 5306 | DNA POLYMERASE III SUBUNITS GAMMA AND TAU (EC 2.7.7.7) |
| 193 | 194 | RXN01637 | VV0156 | 666 | 4 | DNA POLYMERASE III SUBUNITS GAMMA AND TAU (EC 2.7.7.7) |
| 195 | 196 | F RXA01637 | GR00455 | 312 | 4 | DNA POLYMERASE III SUBUNITS GAMMA AND TAU (EC 2.7.7.7) |
| 197 | 198 | RXA00212 | GR00032 | 12407 | 10848 | DNA LIGASE (EC 6.5.1.2) |
| 199 | 200 | RXA00213 | GR00032 | 12888 | 12316 | DNA LIGASE (EC 6.5.1.2) |
| 201 | 202 | RXA00789 | GR00209 | 1266 | 1024 | EXODEOXYRIBONUCLEASE SMALL SUBUNIT (EC 3.1.11.6) |
| 203 | 204 | RXN00790 | VV0321 | 1983 | 3044 | EXODEOXYRIBONUCLEASE LARGE SUBUNIT (EC 3.1.11.6) |
| 205 | 206 | F RXA00790 | GR00209 | 2315 | 1290 | EXODEOXYRIBONUCLEASE LARGE SUBUNIT (EC 3.1.11.6) |
| 207 | 208 | RXA00898 | GR00245 | 567 | 1355 | EXODEOXYRIBONUCLEASE III (EC 3.1.11.2) |
| 209 | 210 | RXN03175 | VV0331 | 1248 | 466 | EXODEOXYRIBONUCLEASE III (EC 3.1.11.2) |
| 211 | 212 | F RXA02883 | GR10020 | 779 | 1690 | EXODEOXYRIBONUCLEASE III (EC 3.1.11.2) |
| 213 | 214 | RXA00341 | GR00059 | 3584 | 4990 | EXORIBONUCLEASE II (EC 3.1.13.1) |
| 215 | 216 | RXA02077 | GR00628 | 7779 | 8174 | RIBONUCLEASE III (EC 3.1.26.3) |
| 217 | 218 | RXN01563 | VV0191 | 4413 | 3205 | RIBONUCLEASE D (EC 3.1.26.3) |
| 219 | 220 | F RXA01563 | GR00436 | 1916 | 1107 | RIBONUCLEASE D (EC 3.1.26.3) |
| 221 | 222 | F RXA01713 | GR00485 | 357 | 4 | RIBONUCLEASE D (EC 3.1.26.3) |
| 223 | 224 | RXA02369 | GR00687 | 3191 | 5479 | RIBONUCLEASE E (EC 3.1.4.—) |
| 225 | 226 | RXN02370 | VV0102 | 1338 | 547 | RIBONUCLEASE E (EC 3.1.4.—) |
| 227 | 228 | F RXA02370 | GR00687 | 5463 | 6254 | RIBONUCLEASE E (EC 3.1.4.—) |
| 229 | 230 | RXA01356 | GR00393 | 3731 | 4357 | RIBONUCLEASE HII (EC 3.1.26.4) |
| 231 | 232 | RXN01786 | VV0084 | 11253 | 10570 | RIBONUCLEASE PH (EC 2.7.7.56) |
| 233 | 234 | F RXA01786 | GR00505 | 1878 | 2318 | RIBONUCLEASE PH (EC 2.7.7.56) |
| 235 | 236 | RXN00163 | VV0084 | 10540 | 9923 | RIBONUCLEASE PH (EC 2.7.7.56) |
| 237 | 238 | F RXA00163 | GR00024 | 6432 | 5872 | RIBONUCLEASE PH (EC 2.7.7.56) |
| 239 | 240 | RXA01424 | GR00417 | 988 | 1296 | RIBONUCLEASE P PROTEIN COMPONENT (EC 3.1.26.5) |
| 241 | 242 | RXA01481 | GR00422 | 14596 | 15087 | GUANYL-SPECIFIC RIBONUCLEASE SA3 (EC 3.1.27.3) |
| 243 | 244 | RXN00724 | VV0052 | 1217 | 3193 | ATP-DEPENDENT DNA HELICASE RECG (EC 3.6.1.—) |
| 245 | 246 | RXN01979 | VV0105 | 18093 | 16513 | ATP-DEPENDENT DNA HELICASE RECQ (EC 3.6.1.—) |
| 247 | 248 | RXN01770 | VV0015 | 50449 | 54213 | DNA HELICASE II (EC 3.6.1.—) |
| 249 | 250 | RXN01378 | VV0091 | 8756 | 10510 | Hypothetical ATP-Dependent RNA Helicase |
| 251 | 252 | RXN02131 | VV0100 | 23500 | 25170 | DNA REPAIR HELICASE RAD25 |
| 253 | 254 | RXN01066 | VV0099 | 21112 | 21837 | DNA REPAIR PROTEIN RECO |
| 255 | 256 | RXN01389 | VV0276 | 1350 | 667 | DNA-DIRECTED RNA POLYMERASE BETA′ CHAIN (EC 2.7.7.6) |
| 257 | 258 | RXN02070 | VV0222 | 8319 | 7198 | MRP PROTEIN HOMOLOG |
| 259 | 260 | RXN02082 | VV0318 | 21225 | 24134 | CHROMOSOME SEGREGATION PROTEIN SMC2 |

Ribosomal genes

| | | | | | | |
|---|---|---|---|---|---|---|
| 261 | 262 | RXA01495 | GR00423 | 8970 | 8524 | RIBOSOME-BINDING FACTOR A |
| 263 | 264 | RXA01893 | GR00542 | 871 | 1425 | Ribosome Recycling Factor (RRF) |
| 265 | 266 | RXA01568 | GR00437 | 2764 | 2330 | RIBOSOMAL-PROTEIN-ALANINE ACETYLTRANSFERASE |
| | | | | | | (EC 2.3.1.128) |
| 267 | 268 | RXA01661 | GR00462 | 1132 | 1797 | RRNA METHYLTRANSFERASE (EC 2.1.1.—) |
| 269 | 270 | RXA01581 | GR00440 | 777 | 1589 | RRNA METHYLTRANSFERASE (EC 2.1.1.—) |
| 271 | 272 | RXA00313 | GR00053 | 3992 | 3054 | RRNA METHYLTRANSFERASE (EC 2.1.1.—) |
| 273 | 274 | RXN00460 | VV0086 | 64848 | 64378 | RRNA METHYLTRANSFERASE (EC 2.1.1.—) |
| 275 | 276 | F RXA00460 | GR00116 | 382 | 5 | RRNA METHYLTRANSFERASE (EC 2.1.1.—) |
| 277 | 278 | RXA02179 | GR00641 | 15353 | 14526 | 23S RRNA METHYLTRANSFERASE (EC 2.1.1.—) |
| 279 | 280 | RXA02522 | GR00725 | 311 | 853 | 16S RRNA PROCESSING PROTEIN RIMM |
| 281 | 282 | RXA00717 | GR00188 | 3617 | 4576 | RIBOSOMAL LARGE SUBUNIT PSEUDOURIDINE SYNTHASE B |
| | | | | | | (EC 4.2.1.70) |
| 283 | 284 | RXA02615 | GR00744 | 973 | 338 | RIBOSOMAL-PROTEIN-ALANINE ACETYLTRANSFERASE |
| | | | | | | (EC 2.3.1.128) |
| 285 | 286 | RXN01343 | VV0025 | 33595 | 34302 | LSU ribosomal protein L1P |
| 287 | 288 | F RXA01343 | GR00389 | 13513 | 12863 | LSU ribosomal protein L1P |
| 289 | 290 | RXN01951 | GR00561 | 1218 | 1778 | LSU ribosomal protein L2P |
| 291 | 292 | F RXA01950 | GR00561 | 938 | 1321 | LSU ribosomal protein L2P |
| 293 | 294 | RXA01286 | GR00372 | 730 | 77 | LSU ribosomal protein L3P |
| 295 | 296 | RXA01948 | GR00561 | 3 | 605 | LSU ribosomal protein L1E (= L4P) |
| 297 | 298 | RXN00706 | VV0005 | 2208 | 2780 | LSU ribosomal protein L5P |
| 299 | 300 | F RXA00711 | GR00186 | 660 | 815 | LSU ribosomal protein L5P |
| 301 | 302 | F RXA00706 | GR00185 | 1 | 267 | LSU ribosomal protein L5P |
| 303 | 304 | RXA00695 | GR00181 | 5118 | 5651 | LSU ribosomal protein L6P |
| 305 | 306 | RXA00543 | GR00140 | 579 | 1004 | LSU ribosomal protein L9P |
| 307 | 308 | RXA01335 | GR00389 | 2736 | 2224 | LSU ribosomal protein L10P |
| 309 | 310 | RXN02826 | VV0025 | 33031 | 33465 | LSU ribosomal protein L11P |
| 311 | 312 | F RXA02826 | GR00807 | 67 | 465 | LSU ribosomal protein L11P |
| 313 | 314 | RXA01334 | GR00389 | 2143 | 1760 | LSU ribosomal protein L12P (L7/L12) |
| 315 | 316 | RXA02037 | GR00620 | 1 | 330 | LSU ribosomal protein L13P |
| 317 | 318 | RXA00699 | GR00181 | 6934 | 7377 | LSU ribosomal protein L15P |
| 319 | 320 | RXA02042 | GR00622 | 720 | 1133 | LSU ribosomal protein L16P |
| 321 | 322 | RXA00670 | GR00176 | 2420 | 1932 | LSU ribosomal protein L17P |
| 323 | 324 | RXA00696 | GR00181 | 5658 | 6059 | LSU ribosomal protein L18P |
| 325 | 326 | RXA01353 | GR00393 | 972 | 1310 | LSU ribosomal protein L19P |
| 327 | 328 | RXA02420 | GR00705 | 5954 | 6334 | LSU ribosomal protein L20P |
| 329 | 330 | RXN02371 | VV0102 | 318 | 4 | LSU ribosomal protein L21P |
| 331 | 332 | F RXA02371 | GR00687 | 6483 | 6752 | LSU ribosomal protein L21P |
| 333 | 334 | RXA01949 | GR00561 | 608 | 910 | LSU ribosomal protein L23P |
| 335 | 336 | RXN00709 | VV0005 | 1523 | 1888 | LSU ribosomal protein L24P |
| 337 | 338 | F RXA00709 | GR00186 | 2 | 340 | LSU ribosomal protein L24P |
| 339 | 340 | RXA00710 | GR00186 | 346 | 657 | LSU ribosomal protein L24P |
| 341 | 342 | RXA02635 | GR00748 | 7846 | 8079 | LSU ribosomal protein L28P |
| 343 | 344 | RXA02043 | GR00622 | 1136 | 1276 | LSU ribosomal protein L29P |
| 345 | 346 | RXA00698 | GR00181 | 6742 | 6924 | LSU ribosomal protein L30P |
| 347 | 348 | RXA02633 | GR00748 | 5506 | 5243 | LSU ribosomal protein L31P |
| 349 | 350 | RXA02636 | GR00748 | 8085 | 8246 | LSU ribosomal protein L33P |
| 351 | 352 | RXA01423 | GR00417 | 715 | 855 | LSU ribosomal protein L34P |
| 353 | 354 | RXA02419 | GR00705 | 5699 | 5890 | LSU ribosomal protein L35P |
| 355 | 356 | RXA02190 | GR00642 | 1277 | 2734 | SSU ribosomal protein S1P |
| 357 | 358 | RXN01912 | VV0150 | 876 | 1613 | SSU ribosomal protein S2P |
| 359 | 360 | F RXA01912 | GR00547 | 876 | 1646 | SSU ribosomal protein S2P |
| 361 | 362 | RXA02041 | GR00622 | 1 | 714 | SSU ribosomal protein S3P |
| 363 | 364 | RXA00672 | GR00176 | 4215 | 3613 | SSU ribosomal protein S4P |
| 365 | 366 | RXA00697 | GR00181 | 6103 | 6735 | SSU ribosomal protein S5P |
| 367 | 368 | RXN00545 | VV0079 | 35852 | 36118 | SSU ribosomal protein S6P |
| 369 | 370 | F RXA00545 | GR00141 | 562 | 816 | SSU ribosomal protein S6P |
| 371 | 372 | RXA01279 | GR00369 | 3240 | 2776 | SSU ribosomal protein S7P |
| 373 | 374 | RXA00694 | GR00181 | 4700 | 5095 | SSU ribosomal protein S8P |
| 375 | 376 | RXN02038 | VV0118 | 333 | 701 | SSU ribosomal protein S9P |
| 377 | 378 | F RXA02038 | GR00620 | 333 | 641 | SSU ribosomal protein S9P |
| 379 | 380 | RXA01287 | GR00372 | 1068 | 766 | SSU ribosomal protein S10P |
| 381 | 382 | RXA00673 | GR00176 | 4331 | 4242 | SSU ribosomal protein S11P |
| 383 | 384 | RXA01280 | GR00369 | 3615 | 3250 | SSU ribosomal protein S12P |
| 385 | 386 | RXA02637 | GR00748 | 8253 | 8555 | SSU ribosomal protein S14P |
| 387 | 388 | RXA01487 | GR00423 | 1172 | 906 | SSU ribosomal protein S15P |
| 389 | 390 | RXA02752 | GR00764 | 6709 | 7203 | SSU ribosomal protein S16P |
| 391 | 392 | RXA02389 | GR00695 | 504 | 764 | SSU ribosomal protein S20P |
| 393 | 394 | RXA00671 | GR00176 | 3492 | 2479 | DNA-DIRECTED RNA POLYMERASE ALPHA CHAIN (EC 2.7.7.6) |
| 395 | 396 | RXN02981 | VV0005 | 35599 | 35964 | SSU ribosomal protein S13P |
| 397 | 398 | RXN03139 | VV0129 | 35552 | 35304 | SSU ribosomal protein S18P |
| 399 | 400 | RXN00673 | VV0005 | 35970 | 36371 | SSU ribosomal protein S11P |
| 401 | 402 | RXN00714 | VV0232 | 10755 | 11315 | RIBOSOMAL-PROTEIN-ALANINE ACETYLTRANSFERASE |
| | | | | | | (EC 2.3.1.128) |
| 403 | 404 | RXN00897 | VV0140 | 3721 | 4725 | RIBOSOMAL-PROTEIN-ALANINE ACETYLTRANSFERASE |
| | | | | | | (EC 2.3.1.128) |
| 405 | 406 | RXN01380 | VV0224 | 15361 | 17559 | TEX PROTEIN |

Genes Involved in Pathogenesis

| | | | | | | |
|---|---|---|---|---|---|---|
| 407 | 408 | RXA00157 | GR00023 | 11848 | 10586 | INVASIN 1 |
| 409 | 410 | RXA00208 | GR00032 | 7947 | 7099 | VULNIBACTIN UTILIZATION PROTEIN VIUB |
| 411 | 412 | RXA00967 | GR00967 | 1351 | 989 | VIRULENCE-ASSOCIATED PROTEIN I |
| 413 | 414 | RXA01149 | GR00323 | 2501 | 2758 | VIRULENCE-ASSOCIATED PROTEIN I |
| 415 | 416 | RXA01305 | GR00376 | 4435 | 2570 | SEROTYPE-SPECIFIC ANTIGEN 1 (EC 3.4.21.—) |
| 417 | 418 | RXA01453 | GR00419 | 2655 | 2951 | VIRULENCE-ASSOCIATED PROTEIN A′ |
| 419 | 420 | RXA01824 | GR00516 | 1367 | 2188 | VULNIBACTIN UTILIZATION PROTEIN VIUB |
| 421 | 422 | RXA01832 | GR00516 | 11787 | 10894 | ANNEXIN VII |
| 423 | 424 | RXA02533 | GR00726 | 3775 | 3209 | (D90768) Immunity repressor protein [Escherichia coli] |
| 425 | 426 | RXN02727 | VV0017 | 6287 | 5376 | ANTIGEN 84 |

Nucleases

| | | | | | | |
|---|---|---|---|---|---|---|
| 427 | 428 | RXN01575 | VV0009 | 50041 | 49022 | RIBONUCLEASE HI (EC 3.1.26.4) |
| 429 | 430 | RXN01966 | VV0155 | 5673 | 5017 | OLIGORIBONUCLEASE (EC 3.1.—.—) |

TABLE 2
GENES IDENTIFIED FROM GENBANK

| GenBank ™ Accession No. | Gene Name | Gene Function | Reference |
|---|---|---|---|
| A09073 | ppg | Phosphoenol pyruvate carboxylase | Bachmann, B. et al. “DNA fragment coding for phosphoenolpyruvat |
| | | | corboxylase, recombinant DNA carrying said fragment, strains carrying the |
| | | | recombinant DNA and method for producing L-aminino acids using said |
| | | | strains,” Patent: EP 0358940-A 3 Mar. 21, 1990 |
| A45579, | | Threonine dehydratase | Moeckel, B. et al. “Production of L-isoleucine by means of recombinant |
| A45581, | | | micro-organisms with deregulated threonine dehydratase,” Patent: WO |
| A45583, | | | 9519442-A 5 Jul. 20, 1995 |
| A45585 | | | |
| A45587 | | | |
| AB003132 | murC; ftsQ; ftsZ | | Kobayashi, M. et al. “Cloning, sequencing, and characterization of the ftsZ |
| | | | gene from coryneform bacteria,” Biochem. Biophys. Res. Commun., |
| | | | 236(2): 383-388 (1997) |
| AB015023 | murC; ftsQ | | Wachi, M. et al. “A murC gene from Coryneform bacteria,” Appl. Microbiol. |
| | | | Biotechnol., 51(2): 223-228 (1999) |
| AB018530 | dtsR | | Kimura, E. et al. “Molecular cloning of a novel gene, dtsR, which rescues the |
| | | | detergent sensitivity of a mutant derived from Brevibacterium |
| | | | lactofermentum,” Biosci. Biotechnol. Biochem., 60(10): 1565-1570 (1996) |
| AB018531 | dtsR1; dtsR2 | | |
| AB020624 | murI | D-glutamate racemase | |
| AB023377 | tkt | transketolase | |
| AB024708 | gltB; gltD | Glutamine 2-oxoglutarate aminotransferase | |
| | | large and small subunits | |
| AB025424 | acn | aconitase | |
| AB027714 | rep | Replication protein | |
| AB027715 | rep; aad | Replication protein; aminoglycoside | |
| | | adenyltransferase | |
| AF005242 | argC | N-acetylglutamate-5-semialdehyde | |
| | | dehydrogenase | |
| AF005635 | glnA | Glutamine synthetase | |
| AF030405 | hisF | cyclase | |
| AF030520 | argG | Argininosuccinate synthetase | |
| AF031518 | argF | Ornithine carbamolytransferase | |
| AF036932 | aroD | 3-dehydroquinate dehydratase | |
| AF038548 | pyc | Pyruvate carboxylase | |
| AF038651 | dciAE; apt; rel | Dipeptide-binding protein; adenine | Wehmeier, L. et al. “The role of the Corynebacterium glutamicum rel gene in |
| | | phosphoribosyltransferase; GTP | (p)ppGpp metabolism,” Microbiology, 144: 1853-1862 (1998) |
| | | pyrophosphokinase | |
| AF041436 | argR | Arginine repressor | |
| AF045998 | impA | Inositol monophosphate phosphatase | |
| AF048764 | argH | Argininosuccinate lyase | |
| AF049897 | argC; argJ; argB; | N-acetylglutamylphosphate reductase; | |
| | argD; argF; argR; argG; argH | ornithine acetyltransferase; N- | |
| | | acetylglutamate kinase; acetylornithine | |
| | | transminase; ornithine | |
| | | carbamoyltransferase; arginine repressor; | |
| | | argininosuccinate synthase; | |
| | | argininosuccinate lyase | |
| AF050109 | inhA | Enoyl-acyl carrier protein reductase | |
| AF050166 | hisG | ATP phosphoribosyltransferase | |
| AF051846 | hisA | Phosphoribosylformimino-5-amino-1- | |
| | | phosphoribosyl-4-imidazolecarboxamide | |
| | | isomerase | |
| AF052652 | metA | Homoserine O-acetyltransferase | Park, S. et al. “Isolation and analysis of metA, a methionine biosynthetic gene |
| | | | encoding homoserine acetyltransferase in Corynebacterium glutamicum,” Mol. |
| | | | Cells., 8(3): 286-294 (1998) |
| AF053071 | aroB | Dehydroquinate synthetase | |
| AF060558 | hisH | Glutamine amidotransferase | |
| AF086704 | hisE | Phosphoribosyl-ATP- | |
| | | pyrophosphohydrolase | |
| AF114233 | aroA | 5-enolpyruvylshikimate 3-phosphate | |
| | | synthase | |
| AF116184 | panD | L-aspartate-alpha-decarboxylase precursor | Dusch, N. et al. “Expression of the Corynebacterium glutamicum panD gene |
| | | | encoding L-aspartate-alpha-decarboxylase leads to pantothenate |
| | | | overproduction in Escherichia coli,” Appl. Environ. Microbiol., 65(4)1530-1539 |
| | | | (1999) |
| AF124518 | aroD; aroE | 3-dehydroquinase; shikimate | |
| | | dehydrogenase | |
| AF124600 | aroC; aroK; aroB; pepQ | Chorismate synthase; shikimate kinase; 3- | |
| | | dehydroquinate synthase; putative | |
| | | cytoplasmic peptidase | |
| AF145897 | inhA | | |
| AF145898 | inhA | | |
| AJ001436 | ectP | Transport of ectoine, glycine betaine, | Peter, H. et al. “Corynebacterium glutamicum is equipped with four secondary |
| | | proline | carriers for compatible solutes: Identification, sequencing, and characterization |
| | | | of the proline/ectoine uptake system, ProP, and the ectoine/proline/glycine |
| | | | betaine carrier, EctP,” J. Bacteriol., 180(22): 6005-6012 (1998) |
| AJ004934 | dapD | Tetrahydrodipicolinate succinylase | Wehrmann, A. et al. “Different modes of diaminopimelate synthesis and their |
| | | (incompleteⁱ) | role in cell wall integrity: A study with Corynebacterium glutamicum,” J. Bacteriol., |
| | | | 180(12): 3159-3165 (1998) |
| AJ007732 | ppc; secG; amt; ocd; soxA | Phosphoenolpyruvate-carboxylase; ?; high | |
| | | affinity ammonium uptake protein; putative | |
| | | ornithine-cyclodecarboxylase; sarcosine oxidase | |
| AJ010319 | ftsY, glnB, glnD; srp; amtP | Involved in cell division; PII protein; | Jakoby, M. et al. “Nitrogen regulation in Corynebacterium glutamicum; |
| | | uridylyltransferase (uridylyl-removing | Isolation of genes involved in biochemical characterization of corresponding |
| | | enzmye); signal recognition particle; low | proteins,” FEMS Microbiol., 173(2): 303-310 (1999) |
| | | affinity ammonium uptake protein | |
| AJ132968 | cat | Chloramphenicol aceteyl transferase | |
| AJ224946 | mqo | L-malate: quinone oxidoreductase | Molenaar, D. et al. “Biochemical and genetic characterization of the |
| | | | membrane-associated malate dehydrogenase (acceptor) from Corynebacterium |
| | | | glutamicum,” Eur. J. Biochem., 254(2): 395-403 (1998) |
| AJ238250 | ndh | NADH dehydrogenase | |
| AJ238703 | porA | Porin | Lichtinger, T. et al. “Biochemical and biophysical characterization of the cell |
| | | | wall porin of Corynebacterium glutamicum: The channel is formed by a low |
| | | | molecular mass polypeptide,” Biochemistry, 37(43): 15024-15032 (1998) |
| D17429 | | Transposable element IS31831 | Vertes, A. A. et al. “Isolation and characterization of IS31831, a transposable |
| | | | element from Corynebacterium glutamicum,” Mol. Microbiol., 11(4): 739-746 |
| | | | (1994) |
| D84102 | odhA | 2-oxoglutarate dehydrogenase | Usuda, Y. et al. “Molecular cloning of the Corynebacterium glutamicum |
| | | | (Brevibacterium lactofermentum AJ12036) odhA gene encoding a novel type |
| | | | of 2-oxoglutarate dehydrogenase,” Microbiology, 142: 3347-3354 (1996) |
| E01358 | hdh; hk | Homoserine dehydrogenase; homoserine | Katsumata, R. et al. “Production of L-thereonine and L-isoleucine,” Patent: JP |
| | | kinase | 1987232392-A 1 Oct. 12, 1987 |
| E01359 | | Upstream of the start codon of homoserine | Katsumata, R. et al. “Production of L-thereonine and L-isoleucine,” Patent: JP |
| | | kinase gene | 1987232392-A 2 Oct. 12, 1987 |
| E01375 | | Tryptophan operon | |
| E01376 | trpL; trpE | Leader peptide; anthranilate synthase | Matsui, K. et al. “Tryptophan operon, peptide and protein coded thereby, |
| | | | utilization of tryptophan operon gene expression and production of |
| | | | tryptophan,” Patent: JP 1987244382-A 1 Oct. 24, 1987 |
| E01377 | | Promoter and operator regions of | Matsui, K. et al. “Tryptophan operon, peptide and protein coded thereby, |
| | | tryptophan operon | utilization of tryptophan operon gene expression and production of |
| | | | tryptophan,” Patent: JP 1987244382-A 1 Oct. 24, 1987 |
| E03937 | | Biotin-synthase | Hatakeyama, K. et al. “DNA fragment containing gene capable of coding |
| | | | biotin synthetase and its utilization,” Patent: JP 1992278088-A 1 Oct. 02, 1992 |
| E04040 | | Diamino pelargonic acid aminotransferase | Kohama, K. et al. “Gene coding diaminopelargonic acid aminotransferase and |
| | | | desthiobiotin synthetase and its utilization,” Patent: JP 1992330284-A 1 |
| | | | Nov. 18, 1992 |
| E04041 | | Desthiobiotinsynthetase | Kohama, K. et al. “Gene coding diaminopelargonic acid aminotransferase and |
| | | | desthiobiotin synthetase and its utilization,” Patent: JP 1992330284-A 1 |
| | | | Nov. 18, 1992 |
| E04307 | | Flavum aspartase | Kurusu, Y. et al. “Gene DNA coding aspartase and utilization thereof,” Patent: |
| | | | JP 1993030977-A 1 Feb. 09, 1993 |
| E04376 | | Isocitric acid lyase | Katsumata, R. et al. “Gene manifestation controlling DNA,” Patent: JP |
| | | | 1993056782-A 3 Mar. 09, 1993 |
| E04377 | | Isocitric acid lyase N-terminal fragment | Katsumata, R. et al. “Gene manifestation controlling DNA,” Patent: JP |
| | | | 1993056782-A 3 Mar. 09, 1993 |
| E04484 | | Prephenate dehydratase | Sotouchi, N. et al. “Production of L-phenylalanine by fermentation,” Patent: JP |
| | | | 1993076352-A 2 Mar. 30, 1993 |
| E05108 | | Aspartokinase | Fugono, N. et al. “Gene DNA coding Aspartokinase and its use,” Patent: JP |
| | | | 1993184366-A 1 Jul. 27, 1993 |
| E05112 | | Dihydro-dipichorinate synthetase | Hatakeyama, K. et al. “Gene DNA coding dihydrodipicolinic acid synthetase |
| | | | and its use,” Patent: JP 1993184371-A 1 Jul. 27, 1993 |
| E05776 | | Diaminopimelic acid dehydrogenase | Kobayashi, M. et al. “Gene DNA coding Diaminopimelic acid dehydrogenase |
| | | | and its use,” Patent: JP 1993284970-A 1 Nov. 02, 1993 |
| E05779 | | Threonine synthase | Kohama, K. et al. “Gene DNA coding threanine synthase and its use,” Patent: |
| | | | JP 1993284972-A 1 Nov. 02, 1993 |
| E06110 | | Prephenate dehydratase | Kikuchi, T. et al. “Production of L-phenylalanine by fermentation method,” |
| | | | Patent: JP 1993344881-A 1 Dec. 27, 1993 |
| E06111 | | Mutated Prephenate dehydratase | Kikuchi, T. et al. “Production of L-phenylalanine by fermentation method,” |
| | | | Patent: JP 1993344881-A 1 Dec. 27, 1993 |
| E06146 | | Acetohydroxy acid synthetase | Inui, M. et al. “Gene capable of coding Acetohydroxy acid synthetase and its |
| | | | use,” Patent: JP 1993344893-A 1 Dec. 27, 1993 |
| E06825 | | Aspartokinase | Sugimoto, M. et al. “Mutant aspartokinase gene,” patent: JP 1994062866-A 1 |
| | | | Mar. 08, 1994 |
| E06826 | | Mutated aspartokinase alpha subunit | Sugimoto, M. et al. “Mutant aspartokinase gene,” patent: JP 1994062866-A 1 |
| | | | Mar. 08, 1994 |
| E06827 | | Mutated aspartokinase alpha subunit | Sugimoto, M. et al. “Mutant aspartokinase gene,” patent: JP 1994062866-A 1 |
| | | | Mar. 08, 1994 |
| E07701 | secY | | Honno, N. et al. “Gene DNA participating in integration of membraneous |
| | | | protein to membrane,” Patent: JP 1994169780-A 1 Jun. 21, 1994 |
| E08177 | | Aspartokinase | Sato, Y. et al. “Genetic DNA capable of coding Aspartokinase released from |
| | | | feedback inhibition and its utilization,” Patent: JP 1994261766-A 1 Sep. 20, 1994 |
| E08178, | | Feedback inhibition-released Aspartokinase | Sato, Y. et al. “Genetic DNA capable of coding Aspartokinase released from |
| E08179, | | | feedback inhibition and its utilization,” Patent: JP 1994261766-A 1 Sep. 20, 1994 |
| E08180, | | | |
| E08181, | | | |
| E08182 | | | |
| E08232 | | Acetohydroxy-acid isomeroreductase | Inui, M. et al. “Gene DNA coding acetohydroxy acid isomeroreductase,” |
| | | | Patent: JP 1994277067-A 1 Oct. 04, 1994 |
| E08234 | secE | | Asai, Y. et al. “Gene DNA coding for translocation machinery of protein,” |
| | | | Patent: JP 1994277073-A 1 Oct. 04, 1994 |
| E08643 | | FT aminotransferase and desthiobiotin | Hatakeyama, K. et al. “DNA fragment having promoter function in |
| | | synthetase promoter region | coryneform bacterium,” Patent: JP 1995031476-A 1 Feb. 03, 1995 |
| E08646 | | Biotin synthetase | Hatakeyama, K. et al. “DNA fragment having promoter function in |
| | | | coryneform bacterium,” Patent: JP 1995031476-A 1 Feb. 03, 1995 |
| E08649 | | Aspartase | Kohama, K. et al “DNA fragment having promoter function in coryneform |
| | | | bacterium,” Patent: JP 1995031478-A 1 Feb. 03, 1995 |
| E08900 | | Dihydrodipicolinate reductase | Madori, M. et al. “DNA fragment containing gene coding Dihydrodipicolinate |
| | | | acid reductase and utilization thereof,” Patent: JP 1995075578-A 1 Mar. 20, 1995 |
| E08901 | | Diaminopimelic acid decarboxylase | Madori, M. et al. “DNA fragment containing gene coding Diaminopimelic acid |
| | | | decarboxylase and utilization thereof,” Patent: JP 1995075579-A 1 Mar. 20, 1995 |
| E12594 | | Serine hydroxymethyltransferase | Hatakeyama, K. et al. “Production of L-trypophan,” Patent: JP 1997028391-A |
| | | | 1 Feb. 04, 1997 |
| E12760, | | transposase | Moriya, M. et al. “Amplification of gene using artificial transposon,” Patent: |
| E12759, | | | JP 1997070291-A Mar. 18, 1997 |
| E12758 | | | |
| E12764 | | Arginyl-tRNA synthetase; diaminopimelic | Moriya, M. et al. “Amplification of gene using artificial transposon,” Patent: |
| | | acid decarboxylase | JP 1997070291-A Mar. 18, 1997 |
| E12767 | | Dihydrodipicolinic acid synthetase | Moriya, M. et al. “Amplification of gene using artificial transposon,” Patent: |
| | | | JP 1997070291-A Mar. 18, 1997 |
| E12770 | | aspartokinase | Moriya, M. et al. “Amplification of gene using artificial transposon,” Patent: |
| | | | JP 1997070291-A Mar. 18, 1997 |
| E12773 | | Dihydrodipicolinic acid reductase | Moriya, M. et al. “Amplification of gene using artificial transposon,” Patent: |
| | | | JP 1997070291-A Mar. 18, 1997 |
| E13655 | | Glucose-6-phosphate dehydrogenase | Hatakeyama, K. et al. “Glucose-6-phosphate dehydrogenase and DNA capable |
| | | | of coding the same,” Patent: JP 1997224661-A 1 Sep. 02, 1997 |
| L01508 | IlvA | Threonine dehydratase | Moeckel, B. et al. “Functional and structural analysis of the threonine |
| | | | dehydratase of Corynebacterium glutamicum,” J. Bacteriol., 174: 8065-8072 (1992) |
| L07603 | EC 4.2.1.15 | 3-deoxy-D-arabinoheptulosonate-7- | Chen, C. et al. “The cloning and nucleotide sequence of Corynebacterium |
| | | phosphate synthase | glutamicum 3-deoxy-D-arabinoheptulosonate-7-phosphate synthase gene,” |
| | | | FEMS Microbiol. Lett., 107: 223-230 (1993) |
| L09232 | IlvB; ilvN; ilvC | Acetohydroxy acid synthase large subunit; | Keilhauer, C. et al. “Isoleucine synthesis in Corynebacterium glutamicum: |
| | | Acetohydroxy acid synthase small subunit; | molecular analysis of the ilvB-ilvN-ilvC operon,” J. Bacteriol., 175(17): 5595-5603 (1993) |
| | | Acetohydroxy acid isomeroreductase | |
| L18874 | PtsM | Phosphoenolpyruvate sugar | Fouet, A et al. “Bacillus subtilis sucrose-specific enzyme II of the |
| | | phosphotransferase | phosphotransferase system: expression in Escherichia coli and homology to |
| | | | enzymes II from enteric bacteria,” PNAS USA, 84(24): 8773-8777 (1987); Lee, J. K. |
| | | | et al. “Nucleotide sequence of the gene encoding the Corynebacterium |
| | | | glutamicum mannose enzyme II and analyses of the deduced protein |
| | | | sequence,” FEMS Microbiol. Lett., 119(1-2): 137-145 (1994) |
| L27123 | aceB | Malate synthase | Lee, H-S. et al. “Molecular characterization of aceB, a gene encoding malate |
| | | | synthase in Corynebacterium glutamicum,” J. Microbiol. Biotechnol., |
| | | | 4(4): 256-263 (1994) |
| L27126 | | Pyruvate kinase | Jetten, M. S. et al. “Structural and functional analysis of pyruvate kinase from |
| | | | Corynebacterium glutamicum,” Appl. Environ. Microbiol., 60(7): 2501-2507 (1994) |
| L28760 | aceA | Isocitrate lyase | |
| L35906 | dtxr | Diphtheria toxin repressor | Oguiza, J. A. et al. “Molecular cloning, DNA sequence analysis, and |
| | | | characterization of the Corynebacterium diphtheriae dtxR from Brevibacterium |
| | | | lactofermentum,” J. Bacteriol., 177(2): 465-467 (1995) |
| M13774 | | Prephenate dehydratase | Follettie, M. T. et al. “Molecular cloning and nucleotide sequence of the |
| | | | Corynebacterium glutamicum pheA gene,” J. Bacteriol., 167: 695-702 (1986) |
| M16175 | 5S rRNA | | Park, Y-H. et al. “Phylogenetic analysis of the coryneform bacteria by 56 |
| | | | rRNA sequences,” J. Bacteriol., 169: 1801-1806 (1987) |
| M16663 | trpE | Anthranilate synthase, 5′ end | Sano, K. et al. “Structure and function of the trp operon control regions of |
| | | | Brevibacterium lactofermentum, a glutamic-acid-producing bacterium,” Gene, |
| | | | 52: 191-200 (1987) |
| M16664 | trpA | Tryptophan synthase, 3′end | Sano, K. et al. “Structure and function of the trp operon control regions of |
| | | | Brevibacterium lactofermentum, a glutamic-acid-producing bacterium,” Gene, |
| | | | 52: 191-200 (1987) |
| M25819 | | Phosphoenolpyruvate carboxylase | O'Regan, M. et al. “Cloning and nucleotide sequence of the |
| | | | Phosphoenolpyruvate carboxylase-coding gene of Corynebacterium |
| | | | glutamicum ATCC13032,” Gene, 77(2): 237-251 (1989) |
| M85106 | | 23S rRNA gene insertion sequence | Roller, C. et al. “Gram-positive bacteria with a high DNA G + C content are |
| | | | characterized by a common insertion within their 23S rRNA genes,” J. Gen. |
| | | | Microbiol., 138: 1167-1175 (1992) |
| M85107, | | 23S rRNA gene insertion sequence | Roller, C. et al. “Gram-positive bacteria with a high DNA G + C content are |
| M85108 | | | characterized by a common insertion within their 23S rRNA genes,” J. Gen. |
| | | | Microbiol., 138: 1167-1175 (1992) |
| M89931 | aecD; brnQ; yhbw | Beta C-S lyase; branched-chain amino acid | Rossol, I. et al. “The Corynebacterium glutamicum aecD gene encodes a C-S |
| | | uptake carrier; hypothetical protein yhbw | lyase with alpha, beta-elimination activity that degrades aminoethylcysteine,” |
| | | | J. Bacteriol., 174(9): 2968-2977 (1992); Tauch, A. et al. “Isoleucine uptake in |
| | | | Corynebacterium glutamicum ATCC 13032 is directed by the brnQ gene |
| | | | product,” Arch. Microbiol., 169(4): 303-312 (1998) |
| S59299 | trp | Leader gene (promoter) | Herry, D. M. et al. “Cloning of the trp gene cluster from a tryptophan- |
| | | | hyperproducing strain of Corynebacterium glutamicum: identification of a |
| | | | mutation in the trp leader sequence,” Appl. Environ. Microbiol., 59(3): 791-799 (1993) |
| U11545 | trpD | Anthranilate phosphoribosyltransferase | O'Gara, J. P. and Dunican, L. K. (1994) Complete nucleotide sequence of the |
| | | | Corynebacterium glutamicum ATCC 21850 tpD gene.” Thesis, Microbiology |
| | | | Department, University College Galway, Ireland. |
| U13922 | cglIM; cglIR; clgIIR | Putative type II 5-cytosoine | Schafer, A. et al. “Cloning and characterization of a DNA region encoding a |
| | | methyltransferase; putative type II | stress-sensitive restriction system from Corynebacterium glutamicum ATCC |
| | | restriction endonuclease; putative type I or | 13032 and analysis of its role in intergeneric conjugation with Escherichia |
| | | type III restriction endonuclease | coli,” J. Bacteriol., 176(23): 7309-7319 (1994); Schafer, A. et al. “The |
| | | | Corynebacterium glutamicum cglIM gene encoding a 5-cytosine in an McrBC- |
| | | | deficient Escherichia coli strain,” Gene, 203(2): 95-101 (1997) |
| U14965 | recA | | |
| U31224 | ppx | | Ankri, S. et al. “Mutations in the Corynebacterium glutamicumproline |
| | | | biosynthetic pathway: A natural bypass of the proA step,” J. Bacteriol., |
| | | | 178(15): 4412-4419 (1996) |
| U31225 | proC | L-proline: NADP+ 5-oxidoreductase | Ankri, S. et al. “Mutations in the Corynebacterium glutamicumproline |
| | | | biosynthetic pathway: A natural bypass of the proA step,” J. Bacteriol., |
| | | | 178(15): 4412-4419 (1996) |
| U31230 | obg; proB; unkdh | ?; gamma glutamyl kinase; similar to D- | Ankri, S. et al. “Mutations in the Corynebacterium glutamicumproline |
| | | isomer specific 2-hydroxyacid | biosynthetic pathway: A natural bypass of the proA step,” J. Bacteriol., |
| | | dehydrogenases | 178(15): 4412-4419 (1996) |
| U31281 | bioB | Biotin synthase | Serebriiskii, I. G., “Two new members of the bio B superfamily: Cloning, |
| | | | sequencing and expression of bio B genes of Methylobacillus flagellatum and |
| | | | Corynebacterium glutamicum,” Gene, 175: 15-22 (1996) |
| U35023 | thtR; accBC | Thiosulfate sulfurtransferase; acyl CoA | Jager, W. et al. “A Corynebacterium glutamicum gene encoding a two-domain |
| | | carboxylase | protein similar to biotin carboxylases and biotin-carboxyl-carrier proteins,” |
| | | | Arch. Microbiol., 166(2); 76-82 (1996) |
| U43535 | cmr | Multidrug resistance protein | Jager, W. et al. “A Corynebacterium glutamicum gene conferring multidrug |
| | | | resistance in the heterologous host Escherichia coli,” J. Bacteriol., |
| | | | 179(7): 2449-2451 (1997) |
| U43536 | clpB | Heat shock ATP-binding protein | |
| U53587 | aphA-3 | 3′5″-aminoglycoside phosphotransferase | |
| U89648 | | Corynebacterium glutamicum unidentified | |
| | | sequence involved in histidine biosynthesis, partial sequence | |
| X04960 | trpA; trpB; trpC; trpD; | Tryptophan operon | Matsui, K. et al. “Complete nucleotide and deduced amino acid sequences of |
| | trpE; trpG; trpL | | the Brevibacterium lactofermentum tryptophan operon,” Nucleic Acids Res., |
| | | | 14(24): 10113-10114 (1986) |
| X07563 | lys A | DAP decarboxylase (meso-diaminopimelate | Yeh, P. et al. “Nucleic sequence of the lysA gene of Corynebacterium |
| | | decarboxylase, EC 4.1.1.20) | glutamicum and possible mechanisms for modulation of its expression,” Mol. |
| | | | Gen. Genet., 212(1): 112-119 (1988) |
| X14234 | EC 4.1.1.31 | Phosphoenolpyruvate carboxylase | Eikmanns, B. J. et al. “The Phosphoenolpyruvate carboxylase gene of |
| | | | Corynebacterium glutamicum: Molecular cloning, nucleotide sequence, and |
| | | | expression,” Mol. Gen. Genet., 218(2): 330-339 (1989); Lepiniec, L. et al. |
| | | | “Sorghum Phosphoenolpyruvate carboxylase gene family: structure, function |
| | | | and molecular evolution,” Plant. Mol. Biol., 21 (3): 487-502 (1993) |
| X17313 | fda | Fructose-bisphosphate aldolase | Von der Osten, C. H. et al. “Molecular cloning, nucleotide sequence and fine- |
| | | | structural analysis of the Corynebacterium glutamicum fda gene: structural |
| | | | comparison of C. glutamicum fructose-1,6-biphosphate aldolase to class I and |
| | | | class II aldolases,” Mol. Microbiol., |
| X53993 | dapA | L-2,3-dihydrodipicolinate synthetase (EC | Bonnassie, S. et al. “Nucleic sequence of the dapA gene from |
| | | 4.2.1.52) | Corynebacterium glutamicum,” Nucleic Acids Res., 18(21): 6421 (1990) |
| X54223 | | AttB-related site | Cianciotto, N. et al. “DNA sequence homology between att B-related sites of |
| | | | Corynebacterium diphtheriae, Corynebacterium ulcerans, Corynebacterium |
| | | | glutamicum, and the attP site of lambdacorynephage,” FEMS. Microbiol, |
| | | | Lett., 66: 299-302 (1990) |
| X54740 | argS; lysA | Arginyl-tRNA synthetase; Diaminopimelate | Marcel, T. et al. “Nucleotide sequence and organization of the upstream region |
| | | decarboxylase | of the Corynebacterium glutamicum lysA gene,” Mol. Microbiol., 4(11): 1819-1830 |
| | | | (1990) |
| X55994 | trpL; trpE | Putative leader peptide; anthranilate | Heery, D. M. et al. “Nucleotide sequence of the Corynebacterium glutamicum |
| | | synthase component 1 | trpE gene,” Nucleic Acids Res., 18(23): 7138 (1990) |
| X56037 | thrC | Threonine synthase | Han, K. S. et al. “The molecular structure of the Corynebacterium glutamicum |
| | | | threonine synthase gene,” Mol. Microbiol., 4(10): 1693-1702 (1990) |
| X56075 | attB-related site | Attachment site | Cianciotto, N. et al. “DNA sequence homology between att B-related sites of |
| | | | Corynebacterium diphtheriae, Corynebacterium ulcerans, Corynebacterium |
| | | | glutamicum, and the attP site of lambdacorynephage,” FEMS. Microbiol, |
| | | | Lett., 66: 299-302 (1990) |
| X57226 | lysC-alpha; lysC-beta; asd | Aspartokinase-alpha subunit; | Kalinowski, J. et al. “Genetic and biochemical analysis of the Aspartokinase |
| | | Aspartokinase-beta subunit; aspartate beta | from Corynebacterium glutamicum,” Mol. Microbiol., 5(5): 1197-1204 (1991); |
| | | semialdehyde dehydrogenase | Kalinowski, J. et al. “Aspartokinase genes lysC alpha and lysC beta overlap |
| | | | and are adjacent to the aspertate beta-semialdehyde dehydrogenase gene asd in |
| | | | Corynebacterium glutamicum,” Mol. Gen. Genet., 224(3): 317-324 (1990) |
| X59403 | gap; pgk; tpi | Glyceraldehyde-3-phosphate; | Eikmanns, B. J. “Identification, sequence analysis, and expression of a |
| | | phosphoglycerate kinase; triosephosphate | Corynebacterium glutamicum gene cluster encoding the three glycolytic |
| | | isomerase | enzymes glyceraldehyde-3-phosphate dehydrogenase, 3-phosphoglycerate |
| | | | kinase, and triosephosphate isomeras,” J. Bacteriol., 174(19): 6076-6086 (1992) |
| X59404 | gdh | Glutamate dehydrogenase | Bormann, E. R. et al. “Molecular analysis of the Corynebacterium glutamicum |
| | | | gdh gene encoding glutamate dehydrogenase,” Mol. Microbiol., 6(3): 317-326 (1992) |
| X60312 | lysI | L-lysine permease | Seep-Feldhaus, A. H. et al. “Molecular analysis of the Corynebacterium |
| | | | glutamicum lysI gene involved in lysine uptake,” Mol. Microbiol., 5(12): 2995-3005 (1991) |
| X66078 | cop1 | Ps1 protein | Joliff, G. et al. “Cloning and nucleotide sequence of the csp1 gene encoding |
| | | | PS1, one of the two major secreted proteins of Corynebacterium glutamicum: |
| | | | The deduced N-terminal region of PS1 is similar to the Mycobacterium antigen |
| | | | 85 complex,” Mol. Microbiol., 6(16): 2349-2362 (1992) |
| X66112 | glt | Citrate synthase | Eikmanns, B. J. et al. “Cloning sequence, expression and transcriptional |
| | | | analysis of the Corynebacterium glutamicum gltA gene encoding citrate |
| | | | synthase,” Microbiol., 140: 1817-1828 (1994) |
| X67737 | dapB | Dihydrodipicolinate reductase | |
| X69103 | csp2 | Surface layer protein PS2 | Peyret, J. L. et al. “Characterization of the cspB gene encoding PS2, an ordered |
| | | | surface-layer protein in Corynebacterium glutamicum,” Mol. Microbiol., |
| | | | 9(1): 97-109 (1993) |
| X69104 | | IS3 related insertion element | Bonamy, C. et al. “Identification of IS1206, a Corynebacterium glutamicum |
| | | | IS3-related insertion sequence and phylogenetic analysis,” Mol. Microbiol., |
| | | | 14(3): 571-581 (1994) |
| X70959 | leuA | Isopropylmalate synthase | Patek, M. et al. “Leucine synthesis in Corynebacterium glutamicum: enzyme |
| | | | activities, structure of leuA, and effect of leuA inactivation on lysine |
| | | | synthesis,” Appl. Environ. Microbiol., 60(1): 133-140 (1994) |
| X71489 | icd | Isocitrate dehydrogenase (NADP+) | Eikmanns, B. J. et al. “Cloning sequence analysis, expression, and inactivation |
| | | | of the Corynebacterium glutamicum icd gene encoding isocitrate |
| | | | dehydrogenase and biochemical characterization of the enzyme,” J. Bacteriol., |
| | | | 177(3): 774-782 (1995) |
| X72855 | GDHA | Glutamate dehydrogenase (NADP+) | |
| X75083, | mtrA | 5-methyltryptophan resistance | Heery, D. M. et al. “A sequence from a tryptophan-hyperproducing strain of |
| X70584 | | | Corynebacterium glutamicum encoding resistance to 5-methyltryptophan,” |
| | | | Biochem. Biophys. Res. Commun., 201(3): 1255-1262 (1994) |
| X75085 | recA | | Fitzpatrick, R. et al. “Construction and characterization of recA mutant strains |
| | | | of Corynebacterium glutamicum and Brevibacterium lactofermentum,” Appl. |
| | | | Microbiol. Biotechnol., 42(4): 575-580 (1994) |
| X75504 | aceA; thiX | Partial Isocitrate lyase; ? | Reinscheid, D. J. et al. “Characterization of the isocitrate lyase gene from |
| | | | Corynebacterium glutamicum and biochemical analysis of the enzyme,” J. Bacteriol., |
| | | | 176(12): 3474-3483 (1994) |
| X76875 | | ATPase beta-subunit | Ludwig, W. et al. “Phylogenetic relationships of bacteria based on comparative |
| | | | sequence analysis of elongation factor Tu and ATP-synthase beta-subunit |
| | | | genes,” Antonie Van Leeuwenhoek, 64: 285-305 (1993) |
| X77034 | tuf | Elongation factor Tu | Ludwig, W. et al. “Phylogenetic relationships of bacteria based on comparative |
| | | | sequence analysis of elongation factor Tu and ATP-synthase beta-subunit |
| | | | genes,” Antonie Van Leeuwenhoek, 64: 285-305 (1993) |
| X77384 | recA | | Billman-Jacobe, H. “Nucleotide sequence of a recA gene from |
| | | | Corynebacterium glutamicum,” DNA Seq., 4(6): 403-404 (1994) |
| X78491 | aceB | Malate synthase | Reinscheid, D. J. et al. “Malate synthase from Corynebacterium glutamicum |
| | | | pta-ack operon encoding phosphotransacetylase: sequence analysis,” |
| | | | Microbiology, 140: 3099-3108 (1994) |
| X80629 | 16S rDNA | 16S ribosomal RNA | Rainey, F. A. et al. “Phylogenetic analysis of the genera Rhodococcus and |
| | | | Norcardia and evidence for the evolutionary origin of the genus Norcardia |
| | | | from within the radiation of Rhodococcus species,” Microbiol., 141: 523-528 (1995) |
| X81191 | gluA; gluB; gluC; gluD | Glutamate uptake system | Kronemeyer, W. et al. “Structure of the gluABCD cluster encoding the |
| | | | glutamate uptake system of Corynebacterium glutamicum,” J. Bacteriol., |
| | | | 177(5): 1152-1158 (1995) |
| X81379 | dapE | Succinyldiaminopimelate desuccinylase | Wehrmann, A. et al. “Analysis of different DNA fragments of |
| | | | Corynebacterium glutamicum complementing dapE of Escherichia coli,” |
| | | | Microbiology, 40: 3349-56 (1994) |
| X82061 | 16S rDNA | 16S ribosomal RNA | Ruimy, R. et al. “Phylogeny of the genus Corynebacterium deduced from |
| | | | analyses of small-subunit ribosomal DNA sequences,” Int. J. Syst. Bacteriol., |
| | | | 45(4): 740-746 (1995) |
| X82928 | asd; lysC | Aspartate-semialdehyde dehydrogenase; ? | Serebrijski, I. et al. “Multicopy suppression by asd gene and osmotic stress- |
| | | | dependent complementation by heterologous proA in proA mutants,” J. Bacteriol., |
| | | | 177(24): 7255-7260 (1995) |
| X82929 | proA | Gamma-glutamyl phosphate reductase | Serebrijski, I. et al. “Multicopy suppression by asd gene and osmotic stress- |
| | | | dependent complementation by heterologous proA in proA mutants,” J. Bacteriol., |
| | | | 177(24): 7255-7260 (1995) |
| X84257 | 16S rDNA | 165 ribosomal RNA | Pascual, C. et al. “Phylogenetic analysis of the genus Corynebacterium based |
| | | | on 16S rRNA gene sequences,” Int. J. Syst. Bacteriol., 45(4): 724-728 (1995) |
| X85965 | aroP; dapE | Aromatic amino acid permease; ? | Wehrmann, A. et al. “Functional analysis of sequences adjacent to dapE of |
| | | | Corynebacterium glutamicumproline reveals the presence of aroP, which |
| | | | encodes the aromatic amino acid transporter,” J. Bacteriol., 177(20): 5991-5993 |
| | | | (1995) |
| X86157 | argB; argC; argD; | Acetylglutamate kinase; N-acetyl-gamma- | Sakanyan, V. et al. “Genes and enzymes of the acetyl cycle of arginine |
| | argF; argJ | glutamyl-phosphate reductase; | biosynthesis in Corynebacterium glutamicum: enzyme evolution in the early |
| | | acetylornithine aminotransferase; ornithine | steps of the arginine pathway,” Microbiology, 142: 99-108 (1996) |
| | | carbamoyltransferase; glutamate N- | |
| | | acetyltransferase | |
| X89084 | pta; ackA | Phosphate acetyltransferase; acetate kinase | Reinscheid, D. J. et al. “Cloning, sequence analysis, expression and inactivation |
| | | | of the Corynebacterium glutamicum pta-ack operon encoding |
| | | | phosphotransacetylase and acetate kinase,” Microbiology, 145: 503-513 (1999) |
| X89850 | attB | Attachment site | Le Marrec, C. et al. “Genetic characterization of site-specific integration |
| | | | functions of phi AAU2 infecting “Arthrobacter aureus C70,” J. Bacteriol., |
| | | | 178(7): 1996-2004 (1996) |
| X90356 | | Promoter fragment F1 | Patek, M. et al. “Promoters from Corynebacterium glutamicum: cloning, |
| | | | molecular analysis and search for a consensus motif,” Microbiology, |
| | | | 142: 1297-1309 (1996) |
| X90357 | | Promoter fragment F2 | Patek, M. et al. “Promoters from Corynebacterium glutamicum: cloning, |
| | | | molecular analysis and search for a consensus motif,” Microbiology, |
| | | | 142: 1297-1309 (1996) |
| X90358 | | Promoter fragment F10 | Patek, M. et al. “Promoters from Corynebacterium glutamicum: cloning, |
| | | | molecular analysis and search for a consensus motif,” Microbiology, |
| | | | 142: 1297-1309 (1996) |
| X90359 | | Promoter fragment F13 | Patek, M. et al. “Promoters from Corynebacterium glutamicum: cloning, |
| | | | molecular analysis and search for a consensus motif,” Microbiology, |
| | | | 142: 1297-1309 (1996) |
| X90360 | | Promoter fragment F22 | Patek, M. et al. “Promoters from Corynebacterium glutamicum: cloning, |
| | | | molecular analysis and search for a consensus motif,” Microbiology, |
| | | | 142: 1297-1309 (1996) |
| X90361 | | Promoter fragment F34 | Patek, M. et al. “Promoters from Corynebacterium glutamicum: cloning, |
| | | | molecular analysis and search for a consensus motif,” Microbiology, |
| | | | 142: 1297-1309 (1996) |
| X90362 | | Promoter fragment F37 | Patek, M. et al. “Promoters from Corynebacterium glutamicum: cloning, |
| | | | molecular analysis and search for a consensus motif,” Microbiology, |
| | | | 142: 1297-1309 (1996) |
| X90363 | | Promoter fragment F45 | Patek, M. et al. “Promoters from Corynebacterium glutamicum: cloning, |
| | | | molecular analysis and search for a consensus motif,” Microbiology, |
| | | | 142: 1297-1309 (1996) |
| X90364 | | Promoter fragment F64 | Patek, M. et al. “Promoters from Corynebacterium glutamicum: cloning, |
| | | | molecular analysis and search for a consensus motif,” Microbiology, |
| | | | 142: 1297-1309 (1996) |
| X90365 | | Promoter fragment F75 | Patek, M. et al. “Promoters from Corynebacterium glutamicum: cloning, |
| | | | molecular analysis and search for a consensus motif,” Microbiology, |
| | | | 142: 1297-1309 (1996) |
| X90366 | | Promoter fragment PF101 | Patek, M. et al. “Promoters from Corynebacterium glutamicum: cloning, |
| | | | molecular analysis and search for a consensus motif,” Microbiology, |
| | | | 142: 1297-1309 (1996) |
| X90367 | | Promoter fragment PF104 | Patek, M. et al. “Promoters from Corynebacterium glutamicum: cloning, |
| | | | molecular analysis and search for a consensus motif,” Microbiology, |
| | | | 142: 1297-1309 (1996) |
| X90368 | | Promoter fragment PF109 | Patek, M. et al. “Promoters from Corynebacterium glutamicum: cloning, |
| | | | molecular analysis and search for a consensus motif,” Microbiology, |
| | | | 142: 1297-1309 (1996) |
| X93513 | amt | Ammonium transport system | Siewe, R. M. et al. “Functional and genetic characterization of the (methyl) |
| | | | ammonium uptake carrier of Corynebacterium glutamicum,” J. Biol. Chem., |
| | | | 271(10): 5398-5403 (1996) |
| X93514 | betP | Glycine betaine transport system | Peter, H. et al. “Isolation, characterization, and expression of the |
| | | | Corynebacterium glutamicum betP gene, encoding the transport system for the |
| | | | compatible solute glycine betaine,” J. Bacteriol., 178(17): 5229-5234 (1996) |
| X95649 | orf4 | | Patek, M. et al. “Identification and transcriptional analysis of the dapB-ORF2- |
| | | | dapA-ORF4 operon of Corynebacterium glutamicum, encoding two enzymes |
| | | | involved in L-lysine synthesis,” Biotechnol. Lett., 19: 1113-1117 (1997) |
| X96471 | lysE; lysG | Lysine exporter protein; Lysine export | Vrljic, M. et al. “A new type of transporter with a new type of cellular |
| | | regulator protein | function: L-lysine export from Corynebacterium glutamicum,” Mol. |
| | | | Microbiol., 22(5): 815-826 (1996) |
| X96580 | panB; panC; xylB | 3-methyl-2-oxobutanoate | Sahm, H. et al. “D-pantothenate synthesis in Corynebacterium glutamicum and |
| | | hydroxymethyltransferase; pantoate-beta- | use of panBC and genes encoding L-valine synthesis for D-pantothenate |
| | | alanine ligase; xylulokinase | overproduction,” Appl. Environ. Microbiol., 65(5): 1973-1979 (1999) |
| X96962 | | Insertion sequence IS1207 and transposase | |
| X99289 | | Elongation factor P | Ramos, A. et al. “Cloning, sequencing and expression of the gene encoding |
| | | | elongation factor P in the amino-acid producer Brevibacterium lactofermentum |
| | | | (Corynebacterium glutamicum ATCC 13869),” Gene, 198: 217-222 (1997) |
| Y00140 | thrB | Homoserine kinase | Mateos, L. M. et al. “Nucleotide sequence of the homoserine kinase (thrB) gene |
| | | | of the Brevibacterium lactofermentum,” Nucleic Acids Res., 15(9): 3922 (1987) |
| Y00151 | ddh | Meso-diaminopimelate D-dehydrogenase | Ishino, S. et al. “Nucleotide sequence of the meso-diaminopimelate D- |
| | | (EC 1.4.1.16) | dehydrogenase gene from Corynebacterium glutamicum,” Nucleic Acids Res., |
| | | | 15(9): 3917 (1987) |
| Y00476 | thrA | Homoserine dehydrogenase | Mateos, L. M. et al. “Nucleotide sequence of the homoserine dehydrogenase |
| | | | (thrA) gene of the Brevibacterium lactofermentum,” Nucleic Acids Res., |
| | | | 15(24): 10598 (1987) |
| Y00546 | hom; thrB | Homoserine dehydrogenase; homoserine | Peoples, O. P. et al. “Nucleotide sequence and fine structural analysis of the |
| | | kinase | Corynebacterium glutamicum hom-thrB operon,” Mol. Microbiol., 2(1): 63-72 (1988) |
| Y08964 | murC; ftsQ/divD; ftsZ | UPD-N-acetylmuramate-alanine ligase; | Honrubia, M. P. et al. “Identification, characterization, and chromosomal |
| | | division initiation protein or cell division | organization of the ftsZ gene from Brevibacterium lactofermentum,” Mol. Gen. |
| | | protein; cell division protein | Genet., 259(1): 97-104 (1998) |
| Y09163 | putP | High affinity proline transport system | Peter, H. et al. “Isolation of the putP gene of Corynebacterium |
| | | | glutamicumproline and characterization of a low-affinity uptake system for |
| | | | compatible solutes,” Arch. Microbiol., 168(2): 143-151 (1997) |
| Y09548 | pyc | Pyruvate carboxylase | Peters-Wendisch, P. G. et al. “Pyruvate carboxylase from Corynebacterium |
| | | | glutamicum: characterization, expression and inactivation of the pyc gene,” |
| | | | Microbiology, 144: 915-927 (1998) |
| Y09578 | leuB | 3-isopropylmalate dehydrogenase | Patek, M. et al. “Analysis of the leuB gene from Corynebacterium |
| | | | glutamicum,” Appl. Microbiol. Biotechnol., 50(1): 42-47 (1998) |
| Y12472 | | Attachment site bacteriophage Phi-16 | Moreau, S. et al. “Site-specific integration of corynephage Phi-16: The |
| | | | construction of an integration vector,” Microbiol., 145: 539-548 (1999) |
| Y12537 | proP | Proline/ectoine uptake system protein | Peter, H. et al. “Corynebacterium glutamicum is equipped with four secondary |
| | | | carriers for compatible solutes: Identification, sequencing, and characterization |
| | | | of the proline/ectoine uptake system, ProP, and the ectoine/proline/glycine |
| | | | betaine carrier, EctP,” J. Bacteriol., 180(22): 6005-6012 (1998) |
| Y13221 | glnA | Glutamine synthetase I | Jakoby, M. et al. “Isolation of Corynebacterium glutamicum glnA gene |
| | | | encoding glutamine synthetase I,” FEMS Microbiol. Lett., 154(1): 81-88 (1997) |
| Y16642 | lpd | Dihydrolipoamide dehydrogenase | |
| Y18059 | | Attachment site Corynephage 304L | Moreau, S. et al. “Analysis of the integration functions of &phi; 304L: An |
| | | | integrase module among corynephages,” Virology, 255(1): 150-159 (1999) |
| Z21501 | argS; lysA | Arginyl-tRNA synthetase; diaminopimelate | Oguiza, J. A. et al. “A gene encoding arginyl-tRNA synthetase is located in the |
| | | decarboxylase (partial) | upstream region of the lysA gene in Brevibacterium lactofermentum: |
| | | | Regulation of argS-lysA cluster expression by arginine,” J. Bacteriol., |
| | | | 175(22): 7356-7362 (1993) |
| Z21502 | dapA; dapB | Dihydrodipicolinate synthase; | Pisabarro, A. et al. “A cluster of three genes (dapA, orf2, and dapB) of |
| | | dihydrodipicolinate reductase | Brevibacterium lactofermentum encodes dihydrodipicolinate reductase, and a |
| | | | third polypeptide of unknown function,” J. Bacteriol., 175(9): 2743-2749 (1993) |
| Z29563 | thrC | Threonine synthase | Malumbres, M. et al. “Analysis and expression of the thrC gene of the encoded |
| | | | threonine synthase,” Appl. Environ. Microbiol., 60(7)2209-2219 (1994) |
| Z46753 | 16S rDNA | Gene for 16S ribosomal RNA | |
| Z49822 | sigA | SigA sigma factor | Oguiza, J. A. et al “Multiple sigma factor genes in Brevibacterium |
| | | | lactofermentum: Characterization of sigA and sigB,” J. Bacteriol., 178(2): 550-553 (1996) |
| Z49823 | galE; dtxR | Catalytic activity UDP-galactose 4- | Oguiza, J. A. et al “The galE gene encoding the UDP-galactose 4-epimerase of |
| | | epimerase; diphtheria toxin regulatory | Brevibacterium lactofermentum is coupled transcriptionally to the dmdR |
| | | protein | gene,” Gene, 177: 103-107 (1996) |
| Z49824 | orf1; sigB | ?; SigB sigma factor | Oguiza, J. A. et al “Multiple sigma factor genes in Brevibacterium |
| | | | lactofermentum: Characterization of sigA and sigB,” J. Bacteriol., 178(2): 550-553 (1996) |
| Z66534 | | Transposase | Correia, A. et al. “Cloning and characterization of an IS-like element present in |
| | | | the genome of Brevibacterium lactofermentum ATCC 13869,” Gene, |
| | | | 170(1): 91-94 (1996) |

ⁱA sequence for this gene was published in the indicated reference. However, the sequence obtained by the inventors of the present application is significantly longer than the published version. It is believed that the published version relied on an incorrect start codon, and thus represents only a fragment of the actual coding region.

TABLE 3
Corynebacterium and Brevibacterium Strains Which May be Used in the Practice of the Invention

| Genus | species | ATCC | FERM | NRRL | CECT | NCIMB | CBS | NCTC | DSMZ |
|---|---|---|---|---|---|---|---|---|---|
| Brevibacterium | ammoniagenes | 21054 | | | | | | | |
| Brevibacterium | ammoniagenes | 19350 | | | | | | | |
| Brevibacterium | ammoniagenes | 19351 | | | | | | | |
| Brevibacterium | ammoniagenes | 19352 | | | | | | | |
| Brevibacterium | ammoniagenes | 19353 | | | | | | | |
| Brevibacterium | ammoniagenes | 19354 | | | | | | | |
| Brevibacterium | ammoniagenes | 19355 | | | | | | | |
| Brevibacterium | ammoniagenes | 19356 | | | | | | | |
| Brevibacterium | ammoniagenes | 21055 | | | | | | | |
| Brevibacterium | ammoniagenes | 21077 | | | | | | | |
| Brevibacterium | ammoniagenes | 21553 | | | | | | | |
| Brevibacterium | ammoniagenes | 21580 | | | | | | | |
| Brevibacterium | ammoniagenes | 39101 | | | | | | | |
| Brevibacterium | butanicum | 21196 | | | | | | | |
| Brevibacterium | divaricatum | 21792 | P928 | | | | | | |
| Brevibacterium | flavum | 21474 | | | | | | | |
| Brevibacterium | flavum | 21129 | | | | | | | |
| Brevibacterium | flavum | 21518 | | | | | | | |
| Brevibacterium | flavum | | | B11474 | | | | | |
| Brevibacterium | flavum | | | B11472 | | | | | |
| Brevibacterium | flavum | 21127 | | | | | | | |
| Brevibacterium | flavum | 21128 | | | | | | | |
| Brevibacterium | flavum | 21427 | | | | | | | |
| Brevibacterium | flavum | 21475 | | | | | | | |
| Brevibacterium | flavum | 21517 | | | | | | | |
| Brevibacterium | flavum | 21528 | | | | | | | |
| Brevibacterium | flavum | 21529 | | | | | | | |
| Brevibacterium | flavum | | | B11477 | | | | | |
| Brevibacterium | flavum | | | B11478 | | | | | |
| Brevibacterium | flavum | 21127 | | | | | | | |
| Brevibacterium | flavum | | | B11474 | | | | | |
| Brevibacterium | healii | 15527 | | | | | | | |
| Brevibacterium | ketoglutamicum | 21004 | | | | | | | |
| Brevibacterium | ketoglutamicum | 21089 | | | | | | | |
| Brevibacterium | ketosoreductum | 21914 | | | | | | | |
| Brevibacterium | lactofermentum | | | | 70 | | | | |
| Brevibacterium | lactofermentum | | | | 74 | | | | |
| Brevibacterium | lactofermentum | | | | 77 | | | | |
| Brevibacterium | lactofermentum | 21798 | | | | | | | |
| Brevibacterium | lactofermentum | 21799 | | | | | | | |
| Brevibacterium | lactofermentum | 21800 | | | | | | | |
| Brevibacterium | lactofermentum | 21801 | | | | | | | |
| Brevibacterium | lactofermentum | | | B11470 | | | | | |
| Brevibacterium | lactofermentum | | | B11471 | | | | | |
| Brevibacterium | lactofermentum | 21086 | | | | | | | |
| Brevibacterium | lactofermentum | 21420 | | | | | | | |
| Brevibacterium | lactofermentum | 21086 | | | | | | | |
| Brevibacterium | lactofermentum | 31269 | | | | | | | |
| Brevibacterium | linens | 9174 | | | | | | | |
| Brevibacterium | linens | 19391 | | | | | | | |
| Brevibacterium | linens | 8377 | | | | | | | |
| Brevibacterium | paraffinolyticum | | | | | 11160 | | | |
| Brevibacterium | spec. | | | | | | 717.73 | | |
| Brevibacterium | spec. | | | | | | 717.73 | | |
| Brevibacterium | spec. | 14604 | | | | | | | |
| Brevibacterium | spec. | 21860 | | | | | | | |
| Brevibacterium | spec. | 21864 | | | | | | | |
| Brevibacterium | spec. | 21865 | | | | | | | |
| Brevibacterium | spec. | 21866 | | | | | | | |
| Brevibacterium | spec. | 19240 | | | | | | | |
| Corynebacterium | acetoacidophilum | 21476 | | | | | | | |
| Corynebacterium | acetoacidophilum | 13870 | | | | | | | |
| Corynebacterium | acetoglutamicum | | | B11473 | | | | | |
| Corynebacterium | acetoglutamicum | | | B11475 | | | | | |
| Corynebacterium | acetoglutamicum | 15806 | | | | | | | |
| Corynebacterium | acetoglutamicum | 21491 | | | | | | | |
| Corynebacterium | acetoglutamicum | 31270 | | | | | | | |
| Corynebacterium | acetophilum | | | B3671 | | | | | |
| Corynebacterium | ammoniagenes | 6872 | | | | | | 2399 | |
| Corynebacterium | ammoniagenes | 15511 | | | | | | | |
| Corynebacterium | fujiokense | 21496 | | | | | | | |
| Corynebacterium | glutamicum | 14067 | | | | | | | |
| Corynebacterium | glutamicum | 39137 | | | | | | | |
| Corynebacterium | glutamicum | 21254 | | | | | | | |
| Corynebacterium | glutamicum | 21255 | | | | | | | |
| Corynebacterium | glutamicum | 31830 | | | | | | | |
| Corynebacterium | glutamicum | 13032 | | | | | | | |
| Corynebacterium | glutamicum | 14305 | | | | | | | |
| Corynebacterium | glutamicum | 15455 | | | | | | | |
| Corynebacterium | glutamicum | 13058 | | | | | | | |
| Corynebacterium | glutamicum | 13059 | | | | | | | |
| Corynebacterium | glutamicum | 13060 | | | | | | | |
| Corynebacterium | glutamicum | 21492 | | | | | | | |
| Corynebacterium | glutamicum | 21513 | | | | | | | |
| Corynebacterium | glutamicum | 21526 | | | | | | | |
| Corynebacterium | glutamicum | 21543 | | | | | | | |
| Corynebacterium | glutamicum | 13287 | | | | | | | |
| Corynebacterium | glutamicum | 21851 | | | | | | | |
| Corynebacterium | glutamicum | 21253 | | | | | | | |
| Corynebacterium | glutamicum | 21514 | | | | | | | |
| Corynebacterium | glutamicum | 21516 | | | | | | | |
| Corynebacterium | glutamicum | 21299 | | | | | | | |
| Corynebacterium | glutamicum | 21300 | | | | | | | |
| Corynebacterium | glutamicum | 39684 | | | | | | | |
| Corynebacterium | glutamicum | 21488 | | | | | | | |
| Corynebacterium | glutamicum | 21649 | | | | | | | |
| Corynebacterium | glutamicum | 21650 | | | | | | | |
| Corynebacterium | glutamicum | 19223 | | | | | | | |
| Corynebacterium | glutamicum | 13869 | | | | | | | |
| Corynebacterium | glutamicum | 21157 | | | | | | | |
| Corynebacterium | glutamicum | 21158 | | | | | | | |
| Corynebacterium | glutamicum | 21159 | | | | | | | |
| Corynebacterium | glutamicum | 21355 | | | | | | | |
| Corynebacterium | glutamicum | 31808 | | | | | | | |
| Corynebacterium | glutamicum | 21674 | | | | | | | |
| Corynebacterium | glutamicum | 21562 | | | | | | | |
| Corynebacterium | glutamicum | 21563 | | | | | | | |
| Corynebacterium | glutamicum | 21564 | | | | | | | |
| Corynebacterium | glutamicum | 21565 | | | | | | | |
| Corynebacterium | glutamicum | 21566 | | | | | | | |
| Corynebacterium | glutamicum | 21567 | | | | | | | |
| Corynebacterium | glutamicum | 21568 | | | | | | | |
| Corynebacterium | glutamicum | 21569 | | | | | | | |
| Corynebacterium | glutamicum | 21570 | | | | | | | |
| Corynebacterium | glutamicum | 21571 | | | | | | | |
| Corynebacterium | glutamicum | 21572 | | | | | | | |
| Corynebacterium | glutamicum | 21573 | | | | | | | |
| Corynebacterium | glutamicum | 21579 | | | | | | | |
| Corynebacterium | glutamicum | 19049 | | | | | | | |
| Corynebacterium | glutamicum | 19050 | | | | | | | |
| Corynebacterium | glutamicum | 19051 | | | | | | | |
| Corynebacterium | glutamicum | 19052 | | | | | | | |
| Corynebacterium | glutamicum | 19053 | | | | | | | |
| Corynebacterium | glutamicum | 19054 | | | | | | | |
| Corynebacterium | glutamicum | 19055 | | | | | | | |
| Corynebacterium | glutamicum | 19056 | | | | | | | |
| Corynebacterium | glutamicum | 19057 | | | | | | | |
| Corynebacterium | glutamicum | 19058 | | | | | | | |
| Corynebacterium | glutamicum | 19059 | | | | | | | |
| Corynebacterium | glutamicum | 19060 | | | | | | | |
| Corynebacterium | glutamicum | 19185 | | | | | | | |
| Corynebacterium | glutamicum | 13286 | | | | | | | |
| Corynebacterium | glutamicum | 21515 | | | | | | | |
| Corynebacterium | glutamicum | 21527 | | | | | | | |
| Corynebacterium | glutamicum | 21544 | | | | | | | |
| Corynebacterium | glutamicum | 21492 | | | | | | | |
| Corynebacterium | glutamicum | | | B8183 | | | | | |
| Corynebacterium | glutamicum | | | B8182 | | | | | |
| Corynebacterium | glutamicum | | | B12416 | | | | | |
| Corynebacterium | glutamicum | | | B12417 | | | | | |
| Corynebacterium | glutamicum | | | B12418 | | | | | |
| Corynebacterium | glutamicum | | | B11476 | | | | | |
| Corynebacterium | glutamicum | 21608 | | | | | | | |
| Corynebacterium | lilium | | P973 | | | | | | |
| Corynebacterium | nitrilophilus | 21419 | | | | 11594 | | | |
| Corynebacterium | spec. | | P4445 | | | | | | |
| Corynebacterium | spec. | | P4446 | | | | | | |
| Corynebacterium | spec. | 31088 | | | | | | | |
| Corynebacterium | spec. | 31089 | | | | | | | |
| Corynebacterium | spec. | 31090 | | | | | | | |
| Corynebacterium | spec. | 31090 | | | | | | | |
| Corynebacterium | spec. | 31090 | | | | | | | |
| Corynebacterium | spec. | 15954 | | | | | | | 20145 |
| Corynebacterium | spec. | 21857 | | | | | | | |
| Corynebacterium | spec. | 21862 | | | | | | | |
| Corynebacterium | spec. | 21863 | | | | | | | |

ATCC: American Type Culture Collection, Rockville, MD, USA
FERM: Fermentation Research Institute, Chiba, Japan
NRRL: ARS Culture Collection, Northern Regional Research Laboratory, Peoria, IL, USA
CECT: Coleccion Espanola de Cultivos Tipo, Valencia, Spain
NCIMB: National Collection of Industrial and Marine Bacteria Ltd., Aberdeen, UK
CBS: Centraalbureau voor Schimmelcultures, Baarn, NL
NCTC: National Collection of Type Cultures, London, UK
DSMZ: Deutsche Sammlung von Mikroorganismen und Zellkulturen, Braunschweig, Germany
For reference see Sugawara, H. et al. (1993) World directory of collections of cultures of microorganisms: Bacteria, fungi and yeasts (4^{th} edn), World federation for culture collections world data center on microorganisms, Saimata, Japen.

TABLE 4
ALIGNMENT RESULTS

| | | | | | | | % | |
|---|---|---|---|---|---|---|---|---|
| | | | | | | | Homo- | |
| | length | | | | | | logy | Date of |
| ID # | (NT) | Genbank Hit | Length | Accession | Name of Genbank Hit | Source of Genbank Hit | (GAP) | Deposit |
| rxa00050 | 2319 | GB_BA1:MTCY50 | 36030 | Z77137 | Mycobacterium tuberculosis H37Rv complete genome; segment 55/162. | Mycobacterium tuberculosis | 56,005 | 17-Jun-98 |
| | | GB_BA2:ECOUW67_0 | 110000 | U18997 | Escherichia coli K-12 chromosomal region from 67.4 to 76.0 minutes. | Escherichia coli | 37,165 | 23-Jan-98 |
| | | GB_BA2:AE000397 | 14820 | AE000397 | Escherichia coli K-12 MG1655 section 287 of 400 of the complete genome. | Escherichia coli | 37,165 | 12-Nov-98 |
| rxa00061 | 2790 | GB_BA1:SC7H2 | 42655 | AL109732 | Streptomyces coelicolor cosmid 7H2. | Streptomyces coelicolor A3(2) | 38,650 | 2-Aug-99 |
| | | GB_BA1:MTCY01B2 | 35938 | Z95554 | Mycobacterium tuberculosis H37Rv complete genome; segment 72/162. | Mycobacterium tuberculosis | 57,626 | 17-Jun-98 |
| | | GB_BA1:MSGPOLA | 3157 | L11920 | Mycobacterium tuberculosis DNA polymerase I (polA) gene, complete cds. | Mycobacterium tuberculosis | 57,626 | 16-MAY-1996 |
| rxa00066 | 913 | GB_BA1:MLCB2407 | 35615 | AL023596 | Mycobacterium leprae cosmid B2407. | Mycobacterium leprae | 64,841 | 27-Aug-99 |
| | | GB_BA1:MTV025 | 121125 | AL022121 | Mycobacterium tuberculosis H37Rv complete genome; segment 155/162. | Mycobacterium tuberculosis | 38,785 | 24-Jun-99 |
| | | GB_BA2:SCD25 | 41622 | AL118514 | Streptomyces coelicolor cosmid D25. | Streptomyces coelicolor A3(2) | 60,241 | 21-Sep-99 |
| rxa00095 | 2412 | GB_BA1:MTCY10D7 | 39800 | Z79700 | Mycobacterium tuberculosis H37Rv complete genome; segment 44/162. | Mycobacterium tuberculosis | 60,260 | 17-Jun-98 |
| | | GB_BA1:ECOUW85 | 91414 | M87049 | E. coli genomic sequence of the region from 84.5 to 86.5 minutes. | Escherichia coli | 50,438 | 29-MAY-1995 |
| | | GB_BA2:AF028736 | 4113 | AF028736 | Serratia marcescens site specific recombinase (xerC) and DNA helicase II | Serratia marcescens | 52,153 | 20-Apr-98 |
| | | | | | (UvrD) genes, complete cds. | | | |
| rxa00103 | 4683 | GB_BA1:SC7C7 | 31636 | AL031031 | Streptomyces coelicolor cosmid 7C7. | Streptomyces coelicolor | 38,447 | 6-Jul-98 |
| | | GB_BA1:D90809 | 18451 | D90809 | E. coli genomic DNA, Kohara clone #318(37.2-37.6 min.). | Escherichia coli | 52,004 | 29-MAY-1997 |
| | | GB_BA2:AE000260 | 10677 | AE000260 | Escherichia coli K-12 MG1655 section 150 of 400 of the complete genome. | Escherichia coli | 52,004 | 12-Nov-98 |
| rxa00157 | 1286 | GB_IN2:AC004295 | 84551 | AC004295 | Drosophila melanogaster DNA sequence (P1 DS08374 (D180)), complete | Drosophila melanogaster | 33,359 | 29-Jul-98 |
| | | | | | sequence. | | | |
| | | GB_IN2:AC004295 | 84551 | AC004295 | Drosophila melanogaster DNA sequence (P1 DS08374 (D180)), complete | Drosophila melanogaster | 36,150 | 29-Jul-98 |
| | | | | | sequence. | | | |
| | | GB_BA1:MTCY71 | 42729 | Z92771 | Mycobacterium tuberculosis H37Rv complete genome; segment 141/162. | Mycobacterium tuberculosis | 42,051 | 10-Feb-99 |
| rxa00163 | 741 | GB_HTG2:AC008199 | 124050 | AC008199 | Drosophila melanogaster chromosome 3 clone BACR01K08 (D756) RPCI-98 | Drosophila melanogaster | 35,510 | 2-Aug-99 |
| | | | | | 01.K.8 map 94D-94D strain y; cn bw sp, ***SEQUENCING IN PROGRESS | | | |
| | | | | | ***, 83 unordered pieces. | | | |
| | | GB_HTG2:AC008199 | 124050 | AC008199 | Drosophila melanogaster chromosome 3 clone BACR01K08 (D756) RPCI-98 | Drosophila melanogaster | 35,510 | 2-Aug-99 |
| | | | | | 01.K.8 map 94D-94D strain y; cn bw sp, ***SEQUENCING IN PROGRESS | | | |
| | | | | | ***, 83 unordered pieces. | | | |
| | | GB_BA1:BLNARK | 1421 | Y16671 | Bacillus licheniformis narK, fnr and ywiC genes. | Bacillus licheniformis | 41,975 | 30-Jun-98 |
| rxa00208 | 972 | GB_BA1:CGICD | 3595 | X71489 | C. glutamicum icd gene for monomeric isocitrate dehydrogenase. | Corynebacterium glutamicum | 47,097 | 17-Feb-95 |
| | | GB_PR4:AC005234 | 190753 | AC005234 | Homo sapiens BAC clone NH0436H22 from 2, complete sequence. | Homo sapiens | 39,343 | 4-Feb-99 |
| | | GB_OV:AF116539 | 1063 | AF116539 | Danio rerio invariant chain-like protein 2 (lclp-2) mRNA, complete cds. | Danio rerio | 38,437 | 08-OCT-1999 |
| rxa00212 | 1683 | GB_BA1:MTV012 | 70287 | AL021287 | Mycobacterium tuberculosis H37Rv complete genome; segment 132/162. | Mycobacterium tuberculosis | 39,743 | 23-Jun-99 |
| | | GB_BA1:MLCB637 | 44882 | Z99263 | Mycobacterium leprae cosmid B637. | Mycobacterium leprae | 60,464 | 17-Sep-97 |
| | | GB_BA1:SC8D9 | 38681 | AL035569 | Streptomyces coelicolor cosmid 8D9. | Streptomyces coelicolor | 60,902 | 26-Feb-99 |
| rxa00213 | 696 | GB_GSS15:AQ655024 | 397 | AQ655024 | Sheared DNA-21O17.TR Sheared DNA Trypanosoma brucei genomic clone | Trypanosoma brucei | 40,500 | 22-Jun-99 |
| | | | | | Sheared DNA-21O17, genomic survey sequence. | | | |
| | | GB_HTG1:HSAC000380164296 | | AC000380 | Homo sapiens chromosome 3 clone pDJ70i11, ***SEQUENCING IN | Homo sapiens | 39,466 | 26-MAR-1997 |
| | | | | | PROGRESS***, 2 unordered pieces. | | | |
| | | GB_HTG1:HSAC000380164296 | | AC000380 | Homo sapiens chromosome 3 clone pDJ70i11, ***SEQUENCING IN | Homo sapiens | 39,466 | 26-MAR-1997 |
| | | | | | PROGRESS***, 2 unordered pieces. | | | |
| rxa00214 | 834 | GB_HTG3:AC008391 | 40119 | AC008391 | Homo sapiens chromosomes clone CIT-HSPC_236F12, ***SEQUENCING | Homo sapiens | 34,836 | 3-Aug-99 |
| | | | | | IN PROGRESS***, 64 unordered pieces. | | | |
| | | GB_HTG3:AC008391 | 40119 | AC008391 | Homo sapiens chromosome 5 clone CIT-HSPC_236F12, ***SEQUENCING | Homo sapiens | 34,836 | 3-Aug-99 |
| | | | | | IN PROGRESS***, 64 unordered pieces. | | | |
| | | GB_EST17:AA645151 | 427 | AA645151 | vs72f12.r1 Stratagene mouse skin (#937313) Mus musculus cDNA clone | Mus musculus | 40,376 | 28-OCT-1997 |
| | | | | | IMAGE: 1151855 5′ similar to gb: X67688 TRANSKETOLASE (HUMAN); | | | |
| | | | | | gb: U05809 Mus musculus LAF1 transketolase mRNA, complete cds | | | |
| | | | | | (MOUSE);, mRNA sequence. | | | |
| rxa00313 | 1062 | GB_HTG1:HSMX1_4 | 110000 | AJ011929 | Homo sapiens chromosome 21 clone Cosmids | Homo sapiens | 38,462 | 15-Sep-99 |
| | | | | | 44C5, Q16H18, 14C10, 25D2, 87D5 map 21q22.2, D21S349-MX1, *** | | | |
| | | | | | SEQUENCING IN PROGRESS***, in ordered pieces. | | | |
| | | GB_HTG1:HSMX1_4 | 110000 | AJ011929 | Homo sapiens chromosome 21 clone Cosmids | Homo sapiens | 38,462 | 15-Sep-99 |
| | | | | | 44C5, Q16H18, 14C10, 25D2, 87D5 map 21q22.2, D21S349-MX1, *** | | | |
| | | | | | SEQUENCING IN PROGRESS***, in ordered pieces. | | | |
| | | GB_BA1:MTCY369 | 36850 | Z80226 | Mycobacterium tuberculosis H37Rv complete genome; segment 36/162. | Mycobacterium tuberculosis | 40,401 | 17-Jun-98 |
| rxa00341 | 1530 | GB_PL2:ATAC005560 | 95137 | AC005560 | Arabidopsis thaliana chromosome II BAC F2I9 genomic sequence, complete | Arabidopsis thaliana | 36,045 | 23-OCT-1998 |
| | | | | | sequence. | | | |
| | | GB_PL1:AB010077 | 77380 | AB010077 | Arabidopsis thaliana genomic DNA, chromosome 5, P1 clone: MYH19, | Arabidopsis thaliana | 36,061 | 20-Nov-99 |
| | | | | | complete sequence. | | | |
| | | GB_PL2:ATAC006593 | 79663 | AC006593 | Arabidopsis thaliana chromosome II BAC F16D14 genomic sequence, | Arabidopsis thaliana | 36,936 | 17-MAR-1999 |
| | | | | | complete sequence. | | | |
| rxa00407 | 2787 | GB_BA1:MTV004 | 69350 | AL009198 | Mycobacterium tuberculosis H37Rv complete genome; segment 144/162. | Mycobacterium tuberculosis | 39,401 | 18-Jun-98 |
| | | GB_STS:AF040123 | 328 | AF040123 | Bos taurus microsatellite DVEPC127, sequence tagged site. | Bos taurus | 35,168 | 1-Feb-98 |
| | | GB_STS:AF040123 | 328 | AF040123 | Bos taurus microsatellite DVEPC127, sequence tagged site. | Bos taurus | 35,168 | 1-Feb-98 |
| rxa00414 | 477 | GB_VI:AB024414 | 110637 | AB024414 | Gallid herpesvirus 1 (serotype 2) DNA, UL region complete sequence. | Gallid herpesvirus 1 (serotype | 40,135 | 4-Aug-99 |
| | | | | 2) | | | | |
| | | GB_VI:AB012572 | 16770 | AB012572 | Gallid herpesvirus 1 (serotype 2) UL41 to UL51 genes, complete cds. | Gallid herpesvirus 1 (serotype | 40,135 | 2-Sep-98 |
| | | | | 2) | | | | |
| | | GB_VI:AB024414 | 110637 | AB024414 | Gallid herpesvirus 1 (serotype 2) DNA, UL region complete sequence. | Gallid herpesvirus 1 (serotype | 37,107 | 4-Aug-99 |
| | | | | | | 2) | | |
| rxa00460 | 594 | GB_HTG3:AC008952 | 71200 | AC008952 | Homo sapiens chromosome 5 clone CITB-H1_2337D22, ***SEQUENCING | Homo sapiens | 34,342 | 3-Aug-99 |
| | | | | | IN PROGRESS***, 69 unordered pieces. | | | |
| | | GB_HTG3:AC008952 | 71200 | AC008952 | Homo sapiens chromosome 5 clone CITB-H1_2337D22, ***SEQUENCING | Homo sapiens | 34,342 | 3-Aug-99 |
| | | | | | IN PROGRESS***, 69 unordered pieces. | | | |
| | | GB_HTG2:AC006872 | 145614 | AC006872 | Caenorhabditis elegans clone Y53G8Y, ***SEQUENCING IN PROGRESS | Caenorhabditis elegans | 35,112 | 26-Feb-99 |
| | | | | | ***, 5 unordered pieces. | | | |
| rxa00542 | 798 | GB_BA1:MSGY219 | 38721 | AD000013 | Mycobacterium tuberculosis sequence from clone y219. | Mycobacterium tuberculosis | 38,668 | 10-DEC-1996 |
| | | GB_BA1:MTCY21D4 | 20760 | Z80775 | Mycobacterium tuberculosis H37Rv complete genome; segment 3/262. | Mycobacterium tuberculosis | 53,137 | 24-Jun-99 |
| | | GB_BA1:MSGDNAB | 40571 | L39923 | Mycobacterium leprae cosmid L222 DNA sequence, 27 CDS features. | Mycobacterium leprae | 38,403 | 29-Apr-97 |
| rxa00543 | 549 | GB_PL2:ATAC006922 | 114950 | AC006922 | Arabidopsis thaliana chromosome II BAC T1J8 genomic sequence, complete | Arabidopsis thaliana | 38,787 | 21-MAY-1999 |
| | | | | | sequence. | | | |
| | | GB_PR4:AC005011 | 166069 | AC005011 | Homo sapiens BAC clone GS111G14 from 7q11, complete sequence. | Homo sapiens | 38,246 | 28-Jul-99 |
| | | GB_HTG2:AC007602 | 95333 | AC007602 | Homo sapiens chromosome 16 clone 2D4, ***SEQUENCING IN PROGRESS | Homo sapiens | 33,457 | 20-MAY-1999 |
| | | | | | ***, 76 unordered pieces. | | | |
| rxa00544 | 1653 | GB_BA1:MLCB1913 | 37750 | AL022118 | Mycobacterium leprae cosmid B1913. | Mycobacterium leprae | 37,339 | 27-Aug-99 |
| | | GB_BA1:MSGDNAB | 40571 | L39923 | Mycobacterium leprae cosmid L222 DNA sequence, 27 CDS features. | Mycobacterium leprae | 40,364 | 29-Apr-97 |
| | | GB_BA2:MSM238027 | 17973 | AJ238027 | Mycobacterium smegmatis mps gene. | Mycobacterium smegmatis | 37,141 | 08-OCT-1999 |
| rxa00545 | 390 | GB_HTG3:AC009314 | 204881 | AC009314 | Homo sapiens clone NH0465K04, ***SEQUENCING IN PROGRESS***, 12 | Homo sapiens | 35,128 | 24-Aug-99 |
| | | | | | unordered pieces. | | | |
| | | GB_HTG3:AC009314 | 204881 | AC009314 | Homo sapiens clone NH0465K04, ***SEQUENCING IN PROGRESS***, 12 | Homo sapiens | 35,128 | 24-Aug-99 |
| | | | | | unordered pieces. | | | |
| | | GB_EST36:AI920042 | 696 | AI920042 | 1572 Pine Lambda Zap Xylem library Pinus taeda cDNA clone | Pinus taeda | 33,947 | 29-Jul-99 |
| | | | | | b12_PL3CSUH, mRNA sequence. | | | |
| rxa00562 | 846 | GB_PR3:HS470K1 | 103613 | AL031780 | Human DNA sequence from clone 470K1 on chromosome 6p22.1-23. | Homo sapiens | 34,251 | 23-Nov-99 |
| | | | | | Contains ESTs, STSs, GSSs, genomic markers D6S263 and D6S277, and | | | |
| | | | | | two ca repeat polymorphisms, complete sequence. | | | |
| | | GB_HTG1:HSJ976O13 | 102370 | AL117354 | Homo sapiens chromosome 1 clone RP5-976O13 map p21.2-22.2,*** | Homo sapiens | 34,928 | 25-Nov-99 |
| | | | | | SEQUENCING IN PROGRESS***, in unordered pieces. | | | |
| | | GB_HTG1:HSJ976O13 | 102370 | AL117354 | Homo sapiens chromosome 1 clone RP5-976O13 map p21.2-22.2, | Homo sapiens | 34,928 | 25-Nov-99 |
| | | | | | ***SEQUENCING IN PROGRESS***, in unordered pieces. | | | |
| rxa00625 | 965 | GB_PR4:AC005049 | 106928 | AC005049 | Homo sapiens clone RG023I15, complete sequence. | Homo sapiens | 37,446 | 21-Aug-99 |
| | | GB_HTG2:HSDJ319M7 | 128208 | AL079341 | Homo sapiens chromosome 6 clone RP1-319M7 map p21.1-21.3, *** | Homo sapiens | 35,759 | 30-Nov-99 |
| | | | | | SEQUENCING IN PROGRESS***, in unordered pieces. | | | |
| | | GB_HTG2:HSDJ319M7 | 128208 | AL079341 | Homo sapiens chromosome 6 clone RP1-319M7 map p21.1-21.3, *** | Homo sapiens | 35,759 | 30-Nov-99 |
| | | | | | SEQUENCING IN PROGRESS***, in unordered pieces. | | | |
| rxa00670 | 612 | GB_BA1:MTY13E12 | 43401 | Z95390 | Mycobacterium tuberculosis H37Rv complete genome; segment 147/162. | Mycobacterium tuberculosis | 41,206 | 17-Jun-98 |
| | | GB_BA1:SC6G4 | 41055 | AL031317 | Streptomyces coelicolor cosmid 6G4. | Streptomyces coelicolor | 55,410 | 20-Aug-98 |
| | | GB_HTG3:AC009422 | 140233 | AC009422 | Homo sapiens clone 44_N_8, ***SEQUENCING IN PROGRESS***, 17 | Homo sapiens | 36,394 | 22-Aug-99 |
| | | | | | unordered pieces. | | | |
| rxa00671 | 1137 | GB_BA1:MTY13E12 | 43401 | Z95390 | Mycobacterium tuberculosis H37Rv complete genome; segment 147/162. | Mycobacterium tuberculosis | 38,172 | 17-Jun-98 |
| | | GB_BA1:SC6G4 | 41055 | AL031317 | Streptomyces coelicolor cosmid 6G4. | Streptomyces coelicolor | 67,810 | 20-Aug-98 |
| | | GB_BA1:MLCB1222 | 34714 | AL049491 | Mycobacterium leprae cosmid B1222. | Mycobacterium leprae | 40,541 | 27-Aug-99 |
| rxa00672 | 726 | GB_BA1:MTY13E12 | 43401 | Z95390 | Mycobacterium tuberculosis H37Rv complete genome; segment 147/162. | Mycobacterium tuberculosis | 38,187 | 17-Jun-98 |
| | | GB_BA1:MLCB1222 | 34714 | AL049491 | Mycobacterium leprae cosmid B1222. | Mycobacterium leprae | 37,027 | 27-Aug-99 |
| | | GB_BA1:MBU15140 | 2136 | U15140 | Mycobacterium bovis ribosomal proteins IF-1 (infA), L36 (rpmJ), S13 (rpsM) | Mycobacterium bovis | 71,933 | 28-OCT-1996 |
| | | | | | and S11 (rpsK) genes, complete cds, and S4 (rpsD) gene, partial cds. | | | |
| rxa00673 | 525 | GB_BA1:SC6G4 | 41055 | AL031317 | Streptomyces coelicolor cosmid 6G4. | Streptomyces coelicolor | 63,238 | 20-Aug-98 |
| | | GB_BA1:MTY13E12 | 43401 | Z95390 | Mycobacterium tuberculosis H37Rv complete genome; segment 147/162. | Mycobacterium tuberculosis | 33,461 | 17-Jun-98 |
| | | GB_BA1:MLCB1222 | 34714 | AL049491 | Mycobacterium leprae cosmid B1222. | Mycobacterium leprae | 37,965 | 27-Aug-99 |
| rxa00694 | 519 | GB_BA1:MLSPCOPER | 6858 | X17524 | M. luteus DNA for spectinomycin (spc) operon. | Micrococcus luteus | 68,093 | 07-DEC-1992 |
| | | GB_BA1:SCSECYDNA | 6154 | X83011 | S. coelicolor secY locus DNA. | Streptomyces coelicolor | 74,611 | 02-MAR-1998 |
| | | GB_BA1:MLCB2492 | 37144 | Z98756 | Mycobacterium leprae cosmid B2492. | Mycobacterium leprae | 66,408 | 28-Aug-97 |
| rxa00695 | 657 | GB_BA1:MTCY210 | 36804 | Z84395 | Mycobacterium tuberculosis H37Rv complete genome; segment 34/162. | Mycobacterium tuberculosis | 69,863 | 17-Jun-98 |
| | | GB_BA1:MSGY42 | 36526 | AD000005 | Mycobacterium tuberculosis sequence from clone y42. | Mycobacterium tuberculosis | 38,880 | 03-DEC-1996 |
| | | GB_BA1:SCSECYDNA | 6154 | X83011 | S. coelicolor secY locus DNA. | Streptomyces coelicolor | 67,580 | 02-MAR-1998 |
| rxa00696 | 525 | GB_BA1:SCSECYDNA | 6154 | X83011 | S. coelicolor secY locus DNA. | Streptomyces coelicolor | 65,873 | 02-MAR-1998 |
| | | GB_BA1:MLSPCOPER | 6858 | X17524 | M. luteus DNA for spectinomycin (spc) operon. | Micrococcus luteus | 65,145 | 07-DEC-1992 |
| | | GB_BA1:MTCY210 | 36804 | Z84395 | Mycobacterium tuberculosis H37Rv complete genome; segment 34/162. | Mycobacterium tuberculosis | 65,779 | 17-Jun-98 |
| rxa00697 | 756 | GB_BA1:MTCY210 | 36804 | Z84395 | Mycobacterium tuberculosis H37Rv complete genome; segment 34/162. | Mycobacterium tuberculosis | 67,353 | 17-Jun-98 |
| | | GB_BA1:MSGY42 | 36526 | AD000005 | Mycobacterium tuberculosis sequence from clone y42. | Mycobacterium tuberculosis | 39,516 | 03-DEC-1996 |
| | | GB_BA1:SCSECYDNA | 6154 | X83011 | S. coelicolor secY locus DNA. | Streptomyces coelicolor | 67,764 | 02-MAR-1998 |
| rxa00698 | 306 | GB_BA1:BSUB0019 | 212610 | Z99122 | Bacillus subtilis complete genome (section 19 of 21): from 3597091 to | Bacillus subtilis | 39,465 | 24-Jun-99 |
| | | | | | 3809700 | | | |
| | | GB_IN2:AC008370 | 132171 | AC008370 | Drosophila melanogaster, chromosome 2R, region 44B-44C, BAC clones | Drosophila melanogaster | 32,558 | 3-Aug-99 |
| | | | | | BACR09N11 and BACR40A15, complete sequence. | | | |
| | | GB_HTG2:AC006878 | 159941 | AC006878 | Caenorhabditis elegans clone Y54H5, ***SEQUENCING IN PROGRESS***, | Caenorhabditis elegans | 36,066 | 26-Feb-99 |
| | | | | | 8 unordered pieces. | | | |
| rxa00699 | 567 | GB_BA1:SCSECYDNA | 6154 | X83011 | S. coelicolor secY locus DNA. | Streptomyces coelicolor | 65,009 | 02-MAR-1998 |
| | | GB_BA1:MLSPCOPER | 6858 | X17524 | M. luteus DNA for spectinomycin (spc) operon. | Micrococcus luteus | 61,538 | 07-DEC-1992 |
| | | GB_BA1:MTCY210 | 36804 | Z84395 | Mycobacterium tuberculosis H37Rv complete genome; segment 34/162. | Mycobacterium tuberculosis | 61,989 | 17-Jun-98 |
| rxa00706 | 696 | GB_BA1:MTCY210 | 36804 | Z84395 | Mycobacterium tuberculosis H37Rv complete genome; segment 34/162. | Mycobacterium tuberculosis | 67,960 | 17-Jun-98 |
| | | GB_BA1:MSGY42 | 36526 | AD000005 | Mycobacterium tuberculosis sequence from clone y42. | Mycobacterium tuberculosis | 39,757 | 03-DEC-1996 |
| | | GB_BA1:MLSPCOPER | 6858 | X17524 | M. luteus DNA for spectinomycin (spc) operon. | Micrococcus luteus | 67,686 | 07-DEC-1992 |
| rxa00709 | 489 | GB_BA1:MTCY210 | 36804 | Z84395 | Mycobacterium tuberculosis H37Rv complete genome; segment 34/162. | Mycobacterium tuberculosis | 69,897 | 17-Jun-98 |
| | | GB_BA1:MSGY42 | 36526 | AD000005 | Mycobacterium tuberculosis sequence from clone y42. | Mycobacterium tuberculosis | 41,401 | 03-DEC-1996 |
| | | GB_BA1:MLCB2492 | 37144 | Z98756 | Mycobacterium leprae cosmid B2492. | Mycobacterium leprae | 68,660 | 28-Aug-97 |
| rxa00710 | 435 | GB_BA1:MTCY210 | 36804 | Z84395 | Mycobacterium tuberculosis H37Rv complete genome; segment 34/162. | Mycobacterium tuberculosis | 69,746 | 17-Jun-98 |
| | | GB_BA1:MSGY42 | 36526 | AD000005 | Mycobacterium tuberculosis sequence from clone y42. | Mycobacterium tuberculosis | 37,709 | 03-DEC-1996 |
| | | GB_BA1:MLCB2492 | 37144 | Z98756 | Mycobacterium leprae cosmid B2492. | Mycobacterium leprae | 68,129 | 28-Aug-97 |
| rxa00717 | 1083 | GB_PAT:I78753 | 1187 | I78753 | Sequence 9 from patent US 5693781. | Unknown. | 37,814 | 3-Apr-98 |
| | | GB_PAT:I92042 | 1187 | I92042 | Sequence 9 from patent US 5726299. | Unknown. | 37,814 | 01-DEC-1998 |
| | | GB_BA1:MTCI125 | 37432 | Z98268 | Mycobacterium tuberculosis H37Rv complete genome; segment 76/162. | Mycobacterium tuberculosis | 50,647 | 17-Jun-98 |
| rxa00789 | 366 | GB_PR4:AC007463 | 166892 | AC007463 | Homo sapiens BAC clone NH0244L12 from 2, complete sequence. | Homo sapiens | 41,047 | 22-OCT-1999 |
| | | GB_IN2:AC004420 | 79987 | AC004420 | Drosophila melanogaster DNA sequence (P1 DS02649 (D132)), complete | Drosophila melanogaster | 34,670 | 20-Jun-98 |
| | | | | | sequence. | | | |
| | | GB_BA1:MTV012 | 70287 | AL021287 | Mycobacterium tuberculosis H37Rv complete genome; segment 132/162. | Mycobacterium tuberculosis | 40,331 | 23-Jun-99 |
| rxa00790 | 1185 | GB_EST23:AI085588 | 492 | AI085588 | oy68d10.x1 NCI_CGAP_CLL1 Homo sapiens cDNA clone IMAGE: 1670995 | Homo sapiens | 34,167 | 24-Sep-98 |
| | | | | | 3′, mRNA sequence. | | | |
| | | GB_HTG4:AC010097 | 198863 | AC010097 | Homo sapiens chromosome unknown clone NH0378I16, WORKING DRAFT | Homo sapiens | 33,907 | 29-OCT-1999 |
| | | | | | SEQUENCE, in unordered pieces. | | | |
| | | GB_HTG4:AC010097 | 198863 | AC010097 | Homo sapiens chromosome unknown clone NH0378I16, WORKING DRAFT | Homo sapiens | 33,907 | 29-OCT-1999 |
| | | | | | SEQUENCE, in unordered pieces. | | | |
| rxa00798 | 1278 | GB_BA1:SCH5 | 40544 | AL035636 | Streptomyces coelicolor cosmid H5. | Streptomyces coelicolor | 39,294 | 25-MAR-1999 |
| | | GB_BA1:MTY15C10 | 33050 | Z95436 | Mycobacterium tuberculosis H37Rv complete genome; segment 154/162. | Mycobacterium tuberculosis | 37,619 | 17-Jun-98 |
| | | GB_PAT:AR053877 | 3107 | AR053877 | Sequence 3 from patent US 5834279. | Unknown. | 62,090 | 29-Sep-99 |
| rxa00807 | 1365 | GB_IN1:CELK07E12 | 58949 | U00054 | Caenorhabditis elegans cosmid K07E12. | Caenorhabditis elegans | 38,743 | 11-MAY-1994 |
| | | GB_PL1:GYCPKR | 1179 | D83718 | Glycyrrhiza echinata mRNA for polyketide reductase, complete cds. | Glycyrrhiza echinata | 41,725 | 20-Feb-99 |
| | | GB_EST31:AU062517 | 350 | AU062517 | AU062517 Rice callus Oryza sativa cDNA clone C11644_1A, mRNA | Oryza sativa | 43,386 | 20-MAY-1999 |
| | | | | | sequence. | | | |
| rxa00817 | 2517 | GB_BA1:MTY15C10 | 33050 | Z95436 | Mycobacterium tuberculosis H37Rv complete genome; segment 154/162. | Mycobacterium tuberculosis | 56,778 | 17-Jun-98 |
| | | GB_GSS11:AQ289275 | 682 | AQ289275 | nbxb0034B24r CUGI Rice BAC Library Oryza sativa genomic clone | Oryza sativa | 38,301 | 03-DEC-1998 |
| | | | | | nbxb0034B24r, genomic survey sequence. | | | |
| | | GB_IN1:CEL012469 | 11025 | AJ012469 | Caenorhabditis elegans mRNA for DYS-1 protein, partial. | Caenorhabditis elegans | 37,981 | 16-Nov-98 |
| rxa00823 | 903 | GB_BA1:MTV025 | 121125 | AL022121 | Mycobacterium tuberculosis H37Rv complete genome; segment 155/162. | Mycobacterium tuberculosis | 37,753 | 24-Jun-99 |
| | | GB_PL2:ATFCA0 | 200576 | Z97335 | Arabidopsis thaliana DNA chromosome 4, ESSA I FCA contig fragment | Arabidopsis thaliana | 37,178 | 28-Jun-99 |
| | | | | | No. 0. | | | |
| | | GB_PL2:ATFCA0 | 200576 | Z97335 | Arabidopsis thaliana DNA chromosome 4, ESSA I FCA contig fragment | Arabidopsis thaliana | 37,016 | 28-Jun-99 |
| | | | | | No. 0. | | | |
| rxa00890 | 1422 | GB_BA1:MTCY27 | 27548 | Z95208 | Mycobacterium tuberculosis H37Rv complete genome; segment 104/162. | Mycobacterium tuberculosis | 39,813 | 17-Jun-98 |
| | | GB_BA2:U32716 | 11618 | U32716 | Haemophilus influenzae Rd section 31 of 163 of the complete genome. | Haemophilus influenzae Rd | 39,588 | 29-MAY-1998 |
| | | GB_HTG2:AC006842 | 299015 | AC006842 | Caenorhabditis elegans clone Y104H12X, ***SEQUENCING IN PROGRESS | Caenorhabditis elegans | 36,003 | 24-Feb-99 |
| | | | | | ***, 13 unordered pieces. | | | |
| rxa00898 | 912 | GB_BA1:MSGY423 | 42741 | AD000014 | Mycobacterium tuberculosis sequence from clone y423. | Mycobacterium tuberculosis | 54,945 | 10-DEC-1996 |
| | | GB_BA1:MTCY22G10 | 35420 | Z84724 | Mycobacterium tuberculosis H37Rv complete genome; segment 21/162. | Mycobacterium tuberculosis | 39,532 | 17-Jun-98 |
| | | GB_IN1:CEC12C8 | 23674 | Z81467 | Caenorhabditis elegans cosmid C12C8, complete sequence. | Caenorhabditis elegans | 36,170 | 23-Nov-98 |
| rxa00967 | 411 | GB_HTG2:AC006244 | 92079 | AC006244 | Drosophila melanogaster chromosome 2 clone DS00212 (D463) | Drosophila melanogaster | 38,765 | 30-Jul-99 |
| | | | | | map 60F1-60F2 strain y; cn bw sp, ***SEQUENCING IN PROGRESS***, | | | |
| | | | | | 3 unordered pieces. | | | |
| | | GB_GSS1:CNS00BE0 | 1101 | AL056856 | Drosophila melanogaster genome survey sequence T7 end of BAC # | Drosophila melanogaster | 34,772 | 4-Jun-99 |
| | | | | | BACR23K12 of RPCI-98 library from Drosophila melanogaster (fruit fly), | | | |
| | | | | | genomic survey sequence. | | | |
| | | GB_HTG2:AC006244 | 92079 | AC006244 | Drosophila melanogaster chromosome 2 clone DS00212 (D463) map | Drosophila melanogaster | 38,765 | 30-Jul-99 |
| | | | | | 60F1-60F2 strain y; cn bw sp, ***SEQUENCING IN PROGRESS***, | | | |
| | | | | | 3 unordered pieces. | | | |
| rxa00990 | 488 | GB_GSS8:AQ078675 | 565 | AQ078675 | CIT-HSP-2368L22.TF CIT-HSP Homo sapiens genomic clone 2368L22, | Homo sapiens | 39,200 | 20-Aug-98 |
| | | | | | genomic survey sequence. | | | |
| | | GB_GSS8:AQ040280 | 323 | AQ040280 | CIT-HSP-2328E18.TR CIT-HSP Homo sapiens genomic clone 2328E18, | Homo sapiens | 38,434 | 11-Jul-98 |
| | | | | | genomic survey sequence. | | | |
| | | GB_PL1:ATF16J13 | 107600 | AL049638 | Arabidopsis thaliana DNA chromosome 4, BAC clone F16J13 (ESSA project). | Arabidopsis thaliana | 43,220 | 14-Apr-99 |
| rxa00994 | 451 | GB_EST17:C74578 | 301 | C74578 | C74578 Rice panicle shorter than 3 cm Oryza sativa cDNA clone E31890_1A, | Oryza sativa | 51,174 | 29-Sep-97 |
| | | | | | mRNA sequence. | | | |
| | | GB_EST36:AF155027 | 827 | AF155027 | AF155027 Zebrafish Kidney cDNA random primed, RZPD library no: 576 | Danio rerio | 35,268 | 22-Jul-99 |
| | | | | | Danio rerio cDNA clone CHBOp576D06232Q3 T7 primer, mRNA sequence. | | | |
| | | GB_EST8:AA023112 | 457 | AA023112 | mh66c12.r1 Soares mouse placenta 4NbMP13.5 14.5 Mus musculus cDNA | Mus musculus | 41,869 | 21-Jan-97 |
| | | | | | clone IMAGE: 455926 5′ similar to PIR: S10960 S10960 hypothetical | | | |
| | | | | | protein - bovine;, mRNA sequence. | | | |
| rxa01030 | 1299 | GB_HTG3:AC011344 | 127964 | AC011344 | Homo sapiens chromosome 5 clone CIT-HSPC_287O14, ***SEQUENCING | Homo sapiens | 37,718 | 06-OCT-1999 |
| | | | | | IN PROGRESS***, 36 unordered pieces. | | | |
| | | GB_HTG3:AC011344 | 127964 | AC011344 | Homo sapiens chromosome 5 clone CIT-HSPC_287O14, ***SEQUENCING | Homo sapiens | 37,718 | 06-OCT-1999 |
| | | | | | IN PROGRESS***, 36 unordered pieces. | | | |
| | | GB_BA1:RPXX04 | 237523 | AJ235273 | Rickettsia prowazekii strain Madrid E, complete genome; segment 4/4. | Rickettsia prowazekii | 34,752 | 11-Nov-98 |
| rxa01064 | 759 | GB_EST16:AA584614 | 489 | AA584614 | no08g11.s1 NCI_CGAP_Phe1 Homo sapiens cDNA clone IMAGE: 1100132 | Homo sapiens | 39,059 | 8-Sep-97 |
| | | | | | 3′, mRNA sequence. | | | |
| | | GB_HTG2:AC007720 | 150070 | AC007720 | Homo sapiens clone 31_B_4, ***SEQUENCING IN PROGRESS***, 7 | Homo sapiens | 38,859 | 3-Jun-99 |
| | | | | | unordered pieces. | | | |
| | | GB_HTG2:AC007720 | 150070 | AC007720 | Homo sapiens clone 31_B_4, ***SEQUENCING IN PROGRESS***, 7 | Homo sapiens | 38,859 | 3-Jun-99 |
| | | | | | unordered pieces. | | | |
| rxa01149 | 381 | GB_PR4:AC006971 | 115861 | AC006971 | Homo sapiens PAC clone DJ0791C19 from 7p11.2-q11.21, complete | Homo sapiens | 37,401 | 08-MAY-1999 |
| | | | | | sequence. | | | |
| | | GB_HTG4:AC007347 | 167025 | AC007347 | Homo sapiens chromosome 16 clone RPCI-11_488J11, ***SEQUENCING IN | Homo sapiens | 36,364 | 31-OCT-1999 |
| | | | | | PROGRESS***, 2 ordered pieces. | | | |
| | | GB_HTG4:AC007347 | 167025 | AC007347 | Homo sapiens chromosome 16 clone RPCI-11_488J11, ***SEQUENCING IN | Homo sapiens | 36,364 | 31-OCT-1999 |
| | | | | | PROGRESS***, 2 ordered pieces. | | | |
| rxa01157 | 1705 | GB_BA1:MTCY49 | 39430 | Z73966 | Mycobacterium tuberculosis H37Rv complete genome; segment 93/162. | Mycobacterium tuberculosis | 39,879 | 24-Jun-99 |
| | | GB_BA2:SAU77894 | 2437 | U77894 | Streptomyces avermitilis helicase-like protein gene, complete cds. | Streptomyces avermitilis | 57,648 | 5-Jan-99 |
| | | GB_BA1:SAAJ3310 | 2437 | AJ223310 | Streptomyces avermitilis sab3 gene, complete CDS. | Streptomyces avermitilis | 57,648 | 8-Apr-98 |
| rxa01238 | 1524 | GB_PR4:AC007367 | 197278 | AC007367 | Homo sapiens BAC clone NH0518G12 from 2, complete sequence. | Homo sapiens | 35,130 | 22-OCT-1999 |
| | | GB_PR4:AC007367 | 197278 | AC007367 | Homo sapiens BAC clone NH0518G12 from 2, complete sequence. | Homo sapiens | 38,153 | 22-OCT-1999 |
| | | GB_HTG2:AC008157 | 171758 | AC008157 | Homo sapiens clone 45_P_22, ***SEQUENCING IN PROGRESS***, 9 | Homo sapiens | 35,762 | 28-Jul-99 |
| | | | | | unordered pieces. | | | |
| rxa01255 | 1203 | GB_EST37:AI944834 | 388 | AI944834 | bs06a03.y1 Drosophila melanogaster adult testis library Drosophila | Drosophila melanogaster | 40,310 | 17-Aug-99 |
| | | | | | melanogaster cDNA clone bs06a03 5′, mRNA sequence. | | | |
| | | GB_EST1:T16608 | 235 | T16608 | NIB1546 Normalized infant brain, Bento Soares Homo sapiens cDNA 3′end, | Homo sapiens | 41,277 | 25-Jul-96 |
| | | | | | mRNA sequence. | | | |
| | | GB_EST10:AA141530 | 515 | AA141530 | CK01913.5prime CK Drosophila melanogaster embryo BlueScript Drosophila | Drosophila melanogaster | 38,477 | 29-Nov-98 |
| | | | | | melanogaster cDNA clone CK01913 5prime, mRNA sequence. | | | |
| rxa01279 | 588 | GB_BA1:MLB1790G | 37617 | Z14314 | M. leprae genes rplL, rpoB, rpoC, end, rpsL, rpsG, efg, tuf, rpsJ, rplC for | Mycobacterium leprae | 71,088 | 11-Feb-93 |
| | | | | | ribosomal protein L7, RNA polymerase beta subunit, RNA polymerase beta′ | | | |
| | | | | | subunit, endonuclease, ribosomal protein S7, ribosomal protein S12, | | | |
| | | | | | elongation factor G, elongation factor Tu, ribosomal protein S10, ribosomal | | | |
| | | | | | protein L3 and mkl gene. | | | |
| | | GB_BA1:MSGRPSLG | 1199 | L34681 | Mycobacterium smegmatis ribosomal protein S12 (rpsL) gene, complete cds; | Mycobacterium smegmatis | 76,408 | 23-Feb-95 |
| | | | | | ribosomal protein S7 (rpsG) gene, complete cds. | | | |
| | | GB_BA1:MLUSTROA | 5291 | M17788 | M. luteus str operon encoding ribosomal protein S12 (str or rpsL) ribosomal | Micrococcus luteus | 71,599 | 26-Apr-93 |
| | | | | | protein S7 (rpsG) EF-G (fus) and EF-Tu (tuf). | | | |
| rxa01280 | 489 | GB_BA1:MLB1790G | 37617 | Z14314 | M. leprae genes rplL, rpoB, rpoC, end, rpsL, rpsG, efg, tuf, rpsJ, rplC for | Mycobacterium leprae | 65,644 | 11-Feb-93 |
| | | | | | ribosomal protein L7, RNA polymerase beta subunit, RNA polymerase beta′ | | | |
| | | | | | subunit, endonuclease, ribosomal protein S7, ribosomal protein S12, | | | |
| | | | | | elongation factor G, elongation factor Tu, ribosomal protein S10, ribosomal | | | |
| | | | | | protein L3 and mkl gene. | | | |
| | | GB_BA2:ECOUW67_2 | 110000 | U18997 | Escherichia coli K-12 chromosomal region from 67.4 to 76.0 minutes. | Escherichia coli | 37,037 | 23-Jan-98 |
| | | GB_BA1:MTV040 | 15100 | AL021943 | Mycobacterium tuberculosis H37Rv complete genome; segment 33/162. | Mycobacterium tuberculosis | 65,849 | 17-Jun-98 |
| rxa01286 | 777 | GB_BA1:PRFUSTUF | 2742 | X98830 | P. rosea fus, tuf, rpsJ and rplC genes. | Planobispora rosea | 67,525 | 19-Nov-96 |
| | | GB_BA1:MLCB2492 | 37144 | Z98756 | Mycobacterium leprae cosmid B2492. | Mycobacterium leprae | 67,111 | 28-Aug-97 |
| | | GB_BA1:MTCY210 | 36804 | Z84395 | Mycobacterium tuberculosis H37Rv complete genome; segment 34/162. | Mycobacterium tuberculosis | 67,021 | 17-Jun-98 |
| rxa01287 | 426 | GB_BA1:MTCY210 | 36804 | Z84395 | Mycobacterium tuberculosis H37Rv complete genome; segment 34/162. | Mycobacterium tuberculosis | 70,423 | 17-Jun-98 |
| | | GB_BA1:MSGY42 | 36526 | AD000005 | Mycobacterium tuberculosis sequence from clone y42. | Mycobacterium tuberculosis | 35,749 | 03-DEC-1996 |
| | | GB_BA1:MLB1790G | 37617 | Z14314 | M. leprae genes rplL, rpoB, rpoC, end, rpsL, rpsG, efg, tuf, rpsJ, rplC for | Mycobacterium leprae | 69,104 | 11-Feb-93 |
| | | | | | ribosomal protein L7, RNA polymerase beta subunit, RNA polymerase beta′ | | | |
| | | | | | subunit, endonuclease, ribosomal protein S7, ribosomal protein S12, | | | |
| | | | | | elongation factor G, elongation factor Tu, ribosomal protein S10, ribosomal | | | |
| | | | | | protein L3 and mkl gene. | | | |
| rxa01305 | 1989 | GB_GSS10:AQ242866 | 511 | AQ242866 | HS_2041_A2_E10_T7 CIT Approved Human Genomic Sperm Library D | Homo sapiens | 38,735 | 03-OCT-1998 |
| | | | | | Homo sapiens genomic clone Plate = 2041 Col = 20 Row = I, genomic survey | | | |
| | | | | | sequence. | | | |
| | | GB_PR2:HSU29874 | 6155 | U29874 | Human Flt3 ligand gene and Flt3 ligand alternatively spliced isoform gene, | Homo sapiens | 38,791 | 29-Feb-96 |
| | | | | | complete cds. | | | |
| | | GB_PR2:HSU29874 | 6155 | U29874 | Human Flt3 ligand gene and Flt3 ligand alternatively spliced isoform gene, | Homo sapiens | 36,655 | 29-Feb-96 |
| | | | | | complete cds. | | | |
| rxa01334 | 507 | GB_BA1:MTY20H10 | 35980 | Z92772 | Mycobacterium tuberculosis H37Rv complete genome; segment 31/162. | Mycobacterium tuberculosis | 66,075 | 17-Jun-98 |
| | | GB_BA1:MLB1790G | 37617 | Z14314 | M. leprae genes rplL, rpoB, rpoC, end, rpsL, rpsG, efg, tuf, rpsJ, rplC for | Mycobacterium leprae | 64,969 | 11-Feb-93 |
| | | | | | ribosomal protein L7, RNA polymerase beta subunit, RNA polymerase beta′ | | | |
| | | | | | subunit, endonuclease, ribosomal protein S7, ribosomal protein S12, | | | |
| | | | | | elongation factor G, elongation factor Tu, ribosomal protein S10, ribosomal | | | |
| | | | | | protein L3 and mkl gene. | | | |
| | | GB_BA1:MSGRPLL | 631 | D16310 | M. bovis rplL gene for ribosomal protein L7/L12. | Mycobacterium bovis | 66,598 | 4-Feb-99 |
| rxa01335 | 636 | GB_BA1:SC5F2A | 40105 | AL049587 | Streptomyces coelicolor cosmid 5F2A. | Streptomyces coelicolor | 40,362 | 24-MAY-1999 |
| | | GB_BA1:SC6A5 | 43632 | AL049485 | Streptomyces coelicolor cosmid 6A5. | Streptomyces coelicolor | 40,362 | 24-MAR-1999 |
| | | GB_EST30:AV002771 | 289 | AV002771 | AV002771 Mus musculus C57BL/6J kidney Mus musculus cDNA clone | Mus musculus | 41,667 | 24-Aug-99 |
| | | | | | 0610020F04, mRNA sequence. | | | |
| rxa01343 | 831 | GB_BA1:MTY20H10 | 35980 | Z92772 | Mycobacterium tuberculosis H37Rv complete genome; segment 31/162. | Mycobacterium tuberculosis | 66,908 | 17-Jun-98 |
| | | GB_BA1:SGNUSG | 7235 | X72787 | S. griseus nusG, rplKAJL gene cluster. | Streptomyces griseus | 63,177 | 06-MAY-1998 |
| | | GB_BA1:STMVBRA1 | 7409 | D50624 | Streptomyces virginiae VbrA gene for NusG like protein, SecE like protein and | Streptomyces virginiae | 63,329 | 10-Feb-99 |
| | | | | | ribosomal protein, aspartate aminotransferase and adenosine deaminase, | | | |
| | | | | | complete cds. | | | |
| rxa01353 | 462 | GB_BA1:SC2E1 | 38962 | AL023797 | Streptomyces coelicolor cosmid 2E1. | Streptomyces coelicolor | 65,368 | 4-Jun-98 |
| | | GB_BA2:SKZ86111 | 7860 | Z86111 | Streptomyces lividans rpsP, trmD, rplS, sipW, sipX, sipY, sipZ, mutT genes | Streptomyces lividans | 65,368 | 27-OCT-1999 |
| | | | | | and 4 open reading frames. | | | |
| | | GB_BA1:MTCY274 | 39991 | Z74024 | Mycobacterium tuberculosis H37Rv complete genome; segment 126/162. | Mycobacterium tuberculosis | 37,013 | 19-Jun-98 |
| rxa01356 | 750 | GB_BA1:BSUB0009 | 208780 | Z99112 | Bacillus subtilis complete genome (section 9 of 21): from 1598421 to | Bacillus subtilis | 44,851 | 26-Nov-97 |
| | | | | | 1807200 | | | |
| | | GB_PL2:CRE132478 | 16445 | AJ132478 | Chlamydomonas reinhardtii STF1 gene, partial. | Chlamydomonas reinhardtii | 37,200 | 29-Sep-99 |
| | | GB_GSS4:AQ701168 | 552 | AQ701168 | HS_2129_A2_B06_T7C CIT Approved Human Genomic Sperm Library D | Homo sapiens | 40,541 | 6-Jul-99 |
| | | | | | Homo sapiens genomic clone Plate = 2129 Col = 12 Rowp = C, | | | |
| | | | | | genomic survey sequence. | | | |
| rxa01374 | 1365 | GB_OV:FR24G11 | 34807 | Z93780 | Fugu rubripes genes encoding carbamoyl phosphate synthetase III, myosin | Fugu rubripes | 37,936 | 22-Nov-99 |
| | | | | | light chain, MAP2. | | | |
| | | GB_EST36:AI881479 | 601 | AI881479 | 606069F03.y1 606 —Ear tissue cDNA library from Schmidt lab Zea mays | Zea mays | 38,963 | 21-Jul-99 |
| | | | | | cDNA, mRNA sequence. | | | |
| | | GB_BA2:AE000733 | 15569 | AE000733 | Aquifex aeolicus section 65 of 109 of the complete genome. | Aquifex aeolicus | 35,421 | 25-MAR-1998 |
| rxa01423 | 264 | GB_BA1:SCH24 | 41625 | AL049826 | Streptomyces coelicolor cosmid H24. | Streptomyces coelicolor | 57,854 | 11-MAY-1999 |
| | | GB_BA1:MSGDNAB | 40571 | L39923 | Mycobacterium leprae cosmid L222 DNA sequence, 27 CDS features. | Mycobacterium leprae | 41,634 | 29-Apr-97 |
| | | GB_BA1:MSORIREP | 10430 | X92503 | M. smegmatis origin of replication and genes rpmH, dnaA, dnaN, gnd, recF, | Mycobacterium smegmatis | 39,535 | 26-Aug-97 |
| | | | | | gyrB, gyrA. | | | |
| rxa01424 | 432 | GB_OV:CCU31864 | 2517 | U31864 | Cyprinus carpio stearyl-CoA desaturase mRNA, complete cds. | Cyprinus carpio | 36,946 | 13-Sep-99 |
| | | GB_IN1:DME010641 | 3733 | AJ010641 | Drosophila melanogaster mRNA for Dof protein, transcript II. | Drosophila melanogaster | 36,768 | 9-Feb-99 |
| | | GB_IN1:DME010642 | 4044 | AJ010642 | Drosophila melanogaster mRNA for Dof protein, transcript I, partial. | Drosophila melanogaster | 36,768 | 6-Sep-99 |
| rxa01453 | 420 | GB_BA1:PDENQOURF | 10425 | L02354 | Paracoccus denitrificans NADH dehydrogenase (URF4), (NQO8), (NQO9), | Paracoccus denitrificans | 41,304 | 20-MAY-1993 |
| | | | | | (URF5), (URF6), (NQO10), (NQO11), (NQO12), (NQO13), and (NQO14) | | | |
| | | | | | genes, complete cds's; biotin [acetyl-CoA carboxyl] ligase (birA) gene, | | | |
| | | | | | complete cds. | | | |
| | | GB_BA2:AF108766 | 14548 | AF108766 | Rhodobacter sphaeroides AsmA (asmA) gene, partial cds; YbaU (ybaU), | Rhodobacter sphaeroides | 41,388 | 9-Nov-99 |
| | | | | | anthranilate synthase component I (trpE), YibQ (yibQ), anthranilate synthase | | | |
| | | | | | component II (trpG), anthranilate phosphoribosyltransferase (trpD), | | | |
| | | | | | indole-3-glycerol phosphate synthase (trpC), molybdenum cofactor | | | |
| | | | | | biosynthesis protein C (moaC), molybdenum cofactor biosynthesis protein A | | | |
| | | | | | (moeA), LexA repressor (lexA), and glutamyl t-RNA synthetase (gluS) genes, | | | |
| | | | | | complete cds; and citrate synthase (cisY) gene, partial cds. | | | |
| | | GB_BA1:SCO001205 | 9589 | AJ001205 | Streptomyces coelicolor A3(2) glycogen metabolism clusterl. | Streptomyces coelicolor | 40,554 | 29-MAR-1999 |
| rxa01480 | 2016 | GB_BA2:AF027507 | 5168 | AF027507 | Mycobacterium smegmatis dGTPase (dgt), and primase (dnaG) genes, | Mycobacterium smegmatis | 58,650 | 16-Jan-98 |
| | | | | | complete cds; tRNA-Asn gene, complete sequence. | | | |
| | | GB_BA1:MTCY98 | 31225 | Z83860 | Mycobacterium tuberculosis H37Rv complete genome; segment 103/162. | Mycobacterium tuberculosis | 36,959 | 17-Jun-98 |
| | | GB_PL1:AB009053 | 78145 | AB009053 | Arabidopsis thaliana genomic DNA, chromosome 5, P1 clone: MQB2. | Arabidopsis thaliana | 37,232 | 13-Feb-99 |
| rxa01481 | 615 | GB_HTG1:LMFL5628 | 32914 | AL049187 | Leishmania major chromosome 4 clone L5628 strain Freidlin, *** | Leishmania major | 38,399 | 29-Apr-99 |
| | | | | | SEQUENCING IN PROGRESS***, in unordered pieces. | | | |
| | | GB_HTG1:LMFL5628 | 32914 | AL049187 | Leishmania major chromosome 4 clone L5628 strain Freidlin, *** | Leishmania major | 38,399 | 29-Apr-99 |
| | | | | | SEQUENCING IN PROGRESS***, in unordered pieces. | | | |
| | | GB_PR3:HSJ182D15 | 79576 | AL049612 | Human DNA sequence ***SEQUENCING IN PROGRESS*** from clone | Homo sapiens | 37,438 | 23-Nov-99 |
| | | | | | dJ182D15, complete sequence. | | | |
| rxa01487 | 390 | GB_BA1:MTV002 | 56414 | AL008967 | Mycobacterium tuberculosis H37Rv complete genome; segment 122/162. | Mycobacterium tuberculosis | 38,182 | 17-Jun-98 |
| | | GB_BA1:SC3C3 | 31382 | AL031231 | Streptomyces coelicolor cosmid 3C3. | Streptomyces coelicolor | 56,923 | 10-Aug-98 |
| | | GB_BA1:MLCB22 | 40281 | Z98741 | Mycobacterium leprae cosmid B22. | Mycobacterium leprae | 38,021 | 22-Aug-97 |
| rxa01495 | 570 | GB_PL1:SCSETRP4 | 5020 | X73297 | S. cerevisiae spacer element. | Saccharomyces cerevisiae | 34,921 | 30-DEC-1993 |
| | | GB_PL2:YSCD9476 | 31184 | U28372 | Saccharomyces cerevisiae chromosome IV cosmid 9476. | Saccharomyces cerevisiae | 34,921 | 1-Aug-97 |
| | | GB_PL1:SCNUF1G | 3522 | Z11582 | S. cerevisiae nuf1 gene. | Saccharomyces cerevisiae | 34,921 | 27-MAR-1992 |
| rxa01563 | 1332 | GB_BA1:AB015023 | 2291 | AB015023 | Corynebacterium glutamicum genes for MurC and FtsQ, complete cds. | Corynebacterium glutamicum | 39,017 | 6-Feb-99 |
| | | GB_PR4:HUAC004381 | 213541 | AC004381 | Homo sapiens Chromosome 16 BAC clone CIT987SK-44M2, complete | Homo sapiens | 36,530 | 23-Nov-99 |
| | | | | | sequence. | | | |
| | | GB_BA1:AB015023 | 2291 | AB015023 | Corynebacterium glutamicum genes for MurC and FtsQ, complete cds. | Corynebacterium glutamicum | 40,463 | 6-Feb-99 |
| rxa01581 | 936 | GB_BA1:MTV016 | 53662 | AL021841 | Mycobacterium tuberculosis H37Rv complete genome; segment 143/162. | Mycobacterium tuberculosis | 41,424 | 23-Jun-99 |
| | | GB_EST1:D23343 | 381 | D23343 | RICC2654A Rice callus Oryza sativa cDNA clone C2654_1A, mRNA | Oryza sativa | 37,831 | 8-Jul-99 |
| | | | | | sequence. | | | |
| | | GB_EST17:C73734 | 455 | C73734 | C73734 Rice panicle (longer than 10 cm) Oryza sativa cDNA clone | Oryza sativa | 39,341 | 23-Sep-97 |
| | | | | | E20291_2A, mRNA sequence. | | | |
| rxa01594 | 942 | GB_BA1:MLCB1351 | 38936 | Z95117 | Mycobacterium leprae cosmid B1351. | Mycobacterium leprae | 36,237 | 24-Jun-97 |
| | | GB_BA1:U00021 | 39193 | U00021 | Mycobacterium leprae cosmid L247. | Mycobacterium leprae | 38,553 | 29-Sep-94 |
| | | GB_PL1:AB025606 | 74282 | AB025606 | Arabidopsis thaliana genomic DNA, chromosome 5, BAC clone: F6N7, | Arabidopsis thaliana | 35,699 | 20-Nov-99 |
| | | | | | complete sequence. | | | |
| rxa01637 | 751 | GB_PR3:AF064861 | 133965 | AF064861 | Homo sapiens PAC 128M19 derived from chromosome 21q22.3, containing | Homo sapiens | 37,600 | 2-Jun-98 |
| | | | | | the HMG-14 and CHD5 genes, complete cds, complete sequence. | | | |
| | | GB_HTG2:AC003656_4 | 110000 | AC003656 | Homo sapiens clone P1 C124G1, ***SEQUENCING IN PROGRESS***, 50 | Homo sapiens | 41,137 | 2-Dec-97 |
| | | | | | unordered pieces. | | | |
| | | GB_HTG2:AC003656_4 | 110000 | AC003656 | Homo sapiens clone P1 C124G1, ***SEQUENCING IN PROGRESS***, 50 | Homo sapiens | 41,137 | 2-Dec-97 |
| | | | | | unordered pieces. | | | |
| rxa01661 | 789 | GB_BA1:MTV036 | 24055 | AL021931 | Mycobacterium tuberculosis H37Rv complete genome; segment 19/162. | Mycobacterium tuberculosis | 40,962 | 17-Jun-98 |
| | | GB_BA1:MSGB1970CS | 39399 | L78815 | Mycobacterium leprae cosmid B1970 DNA sequence. | Mycobacterium leprae | 61,370 | 15-Jun-96 |
| | | GB_HTG2:AC006169 | 174288 | AC006169 | Drosophila melanogaster chromosome 3 clone BACR48E09 (D489) RPCI-98 | Drosophila melanogaster | 32,737 | 2-Aug-99 |
| | | | | | 48.E.9 map 61A1-61A4 strain y; cn bw sp, ***SEQUENCING IN | | | |
| | | | | | PROGRESS***, 9 unordered pieces. | | | |
| rxa01683 | 2691 | GB_BA1:MSGYRBA | 6000 | X94224 | M. smegmatis gyrB and gyrA genes. | Mycobacterium smegmatis | 67,476 | 12-Feb-97 |
| | | GB_BA1:MTCY10H4 | 39160 | Z80233 | Mycobacterium tuberculosis H37Rv complete genome; segment 2/162. | Mycobacterium tuberculosis | 66,828 | 17-Jun-98 |
| | | GB_BA1:MSGYRAB | 5119 | X84077 | M. smegmatis gyrB and gyrA genes. | Mycobacterium smegmatis | 67,090 | 13-MAR-1996 |
| rxa01688 | 953 | GB_BA1:MTCY10H4 | 39160 | Z80233 | Mycobacterium tuberculosis H37Rv complete genome; segment 2/162. | Mycobacterium tuberculosis | 74,397 | 17-Jun-98 |
| | | GB_BA1:MSORIREP | 10430 | X92503 | M. smegmatis origin of replication and genes rpmH, dnaA, dnaN, gnd, recF, | Mycobacterium smegmatis | 74,711 | 26-Aug-97 |
| | | | | | gyrB, gyrA. | | | |
| | | GB_BA1:MSGYRAB | 5119 | X84077 | M. smegmatis gyrB and gyrA genes. | Mycobacterium smegmatis | 74,711 | 13-MAR-1996 |
| rxa01689 | 1239 | GB_BA1:MSORIREP | 10430 | X92503 | M. smegmatis origin of replication and genes rpmH, dnaA, dnaN, gnd, recF, | Mycobacterium smegmatis | 63,470 | 26-Aug-97 |
| | | | | | gyrB, gyrA. | | | |
| | | GB_BA1:MSGYRBA | 6000 | X94224 | M. smegmatis gyrB and gyrA genes. | Mycobacterium smegmatis | 62,969 | 12-Feb-97 |
| | | GB_BA1:MSGYRAB | 5119 | X84077 | M. smegmatis gyrB and gyrA genes. | Mycobacterium smegmatis | 62,886 | 13-MAR-1996 |
| rxa01718 | 609 | GB_GSS13:AQ473371 | 688 | AQ473371 | CITBI-E1-2585J18.TR CITBI-E1 Homo sapiens genomic clone 2585J18, | Homo sapiens | 36,976 | 23-Apr-99 |
| | | | | | genomic survey sequence. | | | |
| | | GB_EST33:AV068888 | 264 | AV068888 | AV068888 Mus musculus small intestine C57BL/6J adult Mus musculus cDNA | Mus musculus | 40,000 | 24-Jun-99 |
| | | | | | clone 2010307A09, mRNA sequence. | | | |
| | | GB_HTG3:AC008277 | 204008 | AC008277 | Homo sapiens clone NH0311B14, ***SEQUENCING IN PROGRESS***, 4 | Homo sapiens | 39,130 | 04-OCT-1999 |
| | | | | | unordered pieces. | | | |
| rxa01736 | 2891 | GB_BA1:MTV014 | 58280 | AL021646 | Mycobacterium tuberculosis H37Rv complete genome; segment 137/162. | Mycobacterium tuberculosis | 38,918 | 18-Jun-98 |
| | | GB_PL2:AF156928 | 2290 | AF156928 | Candida albicans folylpolyglutamate synthetase (fpgs) gene, complete cds. | Candida albicans | 34,894 | 22-Jun-99 |
| | | GB_GSS12:AQ421204 | 483 | AQ421204 | RPCI-11-167B4.TJ RPCI-11 Homo sapiens genomic clone RPCI-11-167B4, | Homo sapiens | 39,085 | 23-MAR-1999 |
| | | | | | genomic survey sequence. | | | |
| rxa01739 | 720 | GB_PR3:HS503F6 | 51476 | AL021153 | Homo sapiens DNA sequence from BAC 503F6 on chromosome 22q11.2-12.1. | Homo sapiens | 35,484 | 23-Nov-99 |
| | | | | | Contains EST and STS. | | | |
| | | GB_OM:CFU73207 | 1845 | U73207 | Canis familiaris beta 1 adrenergic receptor (dogbeta1) gene, complete cds. | Canis familiaris | 39,818 | 31-DEC-1997 |
| | | GB_PR3:HS503F6 | 51476 | AL021153 | Homo sapiens DNA sequence from BAC 503F6 on chromosome 22q11.2-12.1. | Homo sapiens | 36,376 | 23-Nov-99 |
| | | | | | Contains EST and STS. | | | |
| rxa01740 | 1545 | GB_BA1:U00016 | 42931 | U00016 | Mycobacterium leprae cosmid B1937. | Mycobacterium leprae | 57,820 | 01-MAR-1994 |
| | | GB_BA2:PAU73505 | 1332 | U73505 | Pseudomonas aeruginosa dihydroorotase (pyrC) gene, complete cds. | Pseudomonas aeruginosa | 39,322 | 13-Nov-98 |
| | | GB_IN1:CEC52G5 | 42842 | Z67881 | Caenorhabditis elegans cosmid C52G5, complete sequence. | Caenorhabditis elegans | 35,267 | 2-Sep-99 |
| rxa01772 | 5061 | GB_BA2:AE001493 | 10792 | AE001493 | Helicobacter pylori, strain J99 section 54 of 132 of the complete genome. | Helicobacter pylori J99 | 46,571 | 20-Jan-99 |
| | | GB_EST5:N28852 | 555 | N28852 | yx59f11.r1 Soares melanocyte 2NbHM Homo sapiens cDNA clone | Homo sapiens | 38,561 | 4-Jan-96 |
| | | | | | IMAGE: 266061 5′, mRNA sequence. | | | |
| | | GB_EST5:N28844 | 628 | N28844 | yx59d11.r1 Soares melanocyte 2NbHM Homo sapiens cDNA clone | Homo sapiens | 38,118 | 4-Jan-96 |
| | | | | | IMAGE: 266037 5′, mRNA sequence. | | | |
| rxa01786 | 807 | GB_PL1:AB006707 | 82315 | AB006707 | Arabidopsis thaliana genomic DNA, chromosome 5, P1 clone: MYC6, | Arabidopsis thaliana | 37,641 | 20-Nov-99 |
| | | | | | complete sequence. | | | |
| | | GB_PR4:AC006324 | 157310 | AC006324 | Homo sapiens clone DJ1164F05, complete sequence. | Homo sapiens | 36,802 | 11-Nov-99 |
| | | GB_PL2:ATU29168 | 2692 | U29168 | Arabidopsis thaliana DNA repair protein Homolog (XPBara) mRNA, complete | Arabidopsis thaliana | 38,808 | 6-Apr-98 |
| | | | | | cds. | | | |
| rxa01824 | 945 | GB_EST19:AA760333 | 371 | AA760333 | vv75b10.r1 Stratagene mouse skin (#937313) Mus musculus cDNA clone | Mus musculus | 44,211 | 23-Jan-98 |
| | | | | | IMAGE: 1228219 5′, mRNA sequence. | | | |
| | | GB_PR2:CNS00YVE | 34105 | AL096807 | Homo sapiens genomic region containing hypervariable minisatellites | Homo sapiens | 36,374 | 11-OCT-1999 |
| | | | | | chromosome 8[8q24.3] of Homo sapiens. | | | |
| | | GB_HTG3:AC008129_0 | 110000 | AC008129 | Homo sapiens clone RPCI11-473H3, ***SEQUENCING IN PROGRESS***, | Homo sapiens | 38,877 | 24-Jul-99 |
| | | | | | 136 unordered pieces. | | | |
| rxa01832 | 1017 | GB_GSS1:CNS006LB | 929 | AL065711 | Drosophila melanogaster genome survey sequence T7 end of BAC # | Drosophila melanogaster | 35,589 | 3-Jun-99 |
| | | | | | BACR13L01 of RPCI-98 library from Drosophila melanogaster (fruit fly), | | | |
| | | | | | genomic survey sequence. | | | |
| | | GB_RO:MUSIGVCA | 153 | M60955 | Mouse Ig germline H-chain D region, 5′ flank. | Mus musculus | 43,421 | 27-Apr-93 |
| | | GB_BA2:AE001014 | 10636 | AE001014 | Archaeoglobus fulgidus section 93 of 172 of the complete genome. | Archaeoglobus fulgidus | 38,377 | 15-DEC-1997 |
| rxa01866 | 421 | GB_IN2:AC008370 | 132171 | AC008370 | Drosophila melanogaster, chromosome 2R, region 44B-44C, BAC clones | Drosophila melanogaster | 34,845 | 3-Aug-99 |
| | | | | | BACR09N11 and BACR40A15, complete sequence. | | | |
| | | GB_IN2:AC008370 | 132171 | AC008370 | Drosophila melanogaster, chromosome 2R, region 44B-44C, BAC clones | Drosophila melanogaster | 37,897 | 3-Aug-99 |
| | | | | | BACR09N11 and BACR40A15, complete sequence. | | | |
| | | GB_HTG1:HSAJ9613 | 45302 | AJ009613 | Homo sapiens chromosome 17 clone cosmid 5L5 map p11, *** | Homo sapiens | 38,717 | 11-Nov-98 |
| | | | | | SEQUENCING IN PROGRESS***, in unordered pieces. | | | |
| rxa01867 | 531 | GB_EST15:AA520493 | 439 | AA520493 | TgESTzz61f08.r1 TgME49 invivo Bradyzoite cDNA size selected Toxoplasma | Toxoplasma gondil | 38,413 | 16-Jul-97 |
| | | | | | gondii cDNA clone tgzz61f08.r1 5′, mRNA sequence. | | | |
| | | GB_GSS10:AQ225693 | 448 | AQ225693 | HS_2009_B2_B08_T7 CIT Approved Human Genomic Sperm Library D | Homo sapiens | 37,374 | 26-Sep-98 |
| | | | | | Homo sapiens genomic clone Plate = 2009 Col = 16 Row = D, genomic survey | | | |
| | | | | | sequence. | | | |
| | | GB_PR3:AC005262 | 44235 | AC005262 | Homo sapiens chromosome 19, cosmid F23990, complete sequence. | Homo sapiens | 34,345 | 6-Jul-98 |
| rxa01876 | 1974 | GB_OV:CHKTNTC | 1185 | M10013 | Chicken cardiac troponin T form I mRNA, complete cds. | Gallus gallus | 41,674 | 28-Apr-93 |
| | | GB_OV:CHKTNT | 927 | K02263 | Chicken troponin T mRNA. | Gallus gallus | 40,065 | 28-Apr-93 |
| | | GB_OV:CHKTNTC | 1185 | M10013 | Chicken cardiac troponin T form I mRNA, complete cds. | Gallus gallus | 42,097 | 28-Apr-93 |
| rxa01893 | 678 | GB_BA1:AB016498 | 596 | AB016498 | TherMus thermophilus frr gene for ribosome recycling factor gene (RRF), | TherMus thermophilus | 53,691 | 9-Apr-99 |
| | | | | | complete cds. | | | |
| | | GB_PR4:AC002531 | 197900 | AC002531 | Homo sapiens chromosome Y, clone_486_O_8, complete sequence. | Homo sapiens | 33,628 | 13-OCT-1999 |
| | | GB_HTG5:AC008019 | 190459 | AC008019 | Mus musculus, ***SEQUENCING IN PROGRESS***, 16 unordered pieces. | Mus musculus | 35,022 | 16-Nov-99 |
| rxa01912 | 861 | GB_BA1:SC2E1 | 38962 | AL023797 | Streptomyces coelicolor cosmid 2E1. | Streptomyces coelicolor | 66,047 | 4-Jun-98 |
| | | GB_BA1:MTCY274 | 39991 | Z74024 | Mycobacterium tuberculosis H37Rv complete genome; segment 126/162. | Mycobacterium tuberculosis | 38,225 | 19-Jun-98 |
| | | GB_BA2:AF034101 | 2162 | AF034101 | Streptomyces coelicolor ribosomal protein S2 (rpsB) and elongation factor Ts | Streptomyces coelicolor | 65,814 | 15-OCT-1999 |
| | | | | | (tsf) genes, complete cds. | | | |
| rxa01948 | 626 | GB_BA1:MSRPLD | 648 | Y13226 | Mycobacterium smegmatis rpID gene. | Mycobacterium smegmatis | 68,833 | 04-DEC-1997 |
| | | GB_BA1:MSGY42 | 36526 | AD000005 | Mycobacterium tuberculosis sequence from clone y42. | Mycobacterium tuberculosis | 35,313 | 03-DEC-1998 |
| | | GB_BA1:MTCY210 | 36804 | Z84395 | Mycobacterium tuberculosis H37Rv complete genome; segment 34/162. | Mycobacterium tuberculosis | 68,530 | 17-Jun-98 |
| rxa01949 | 426 | GB_BA1:MBS10OPER | 5962 | Y13228 | Mycobacterium bovis BCG DNA for ribosomal S10 operon. | Mycobacterium bovis BCG | 66,197 | 04-DEC-1997 |
| | | GB_BA1:MTCY210 | 36804 | Z84395 | Mycobacterium tuberculosis H37Rv complete genome; segment 34/162. | Mycobacterium tuberculosis | 65,962 | 17-Jun-98 |
| | | GB_BA1:MSGY42 | 36526 | AD000005 | Mycobacterium tuberculosis sequence from clone y42. | Mycobacterium tuberculosis | 38,765 | 03-DEC-1996 |
| rxa01951 | 684 | GB_BA1:MTCY210 | 36804 | Z84395 | Mycobacterium tuberculosis H37Rv complete genome; segment 34/162. | Mycobacterium tuberculosis | 71,157 | 17-Jun-98 |
| | | GB_BA1:MSGY42 | 36526 | AD000005 | Mycobacterium tuberculosis sequence from clone y42. | Mycobacterium tuberculosis | 38,179 | 03-DEC-1996 |
| | | GB_BA1:MLCB2492 | 37144 | Z98756 | Mycobacterium leprae cosmid B2492. | Mycobacterium leprae | 70,425 | 28-Aug-97 |
| rxa02037 | 353 | GB_PR4:AC004921 | 150332 | AC004921 | Homo sapiens PAC clone DJ0899E09 from 7q11.23-q21.1, complete | Homo sapiens | 37,822 | 14-Jan-99 |
| | | | | | sequence. | | | |
| | | GB_HTG6:AC008076 | 200000 | AC008076 | Homo sapiens chromosome 4, ***SEQUENCING IN PROGRESS***, 18 | Homo sapiens | 41,860 | 02-DEC-1999 |
| | | | | | unordered pieces. | | | |
| | | GB_PR4:AC004921 | 150332 | AC004921 | Homo sapiens PAC clone DJ0899E09 from 7q11.23-q21.1, complete | Homo sapiens | 37,685 | 14-Jan-99 |
| | | | | | sequence. | | | |
| rxa02038 | 492 | GB_BA2:SCU43429 | 1740 | U43429 | Streptomyces coelicolor ribosomal protein L13 (rplM) and S9 (rpsl) genes, | Streptomyces coelicolor | 55,876 | 13-Jan-98 |
| | | | | | complete cds. | | | |
| | | GB_BA1:SC6G4 | 41055 | AL031317 | Streptomyces coelicolor cosmid 6G4. | Streptomyces coelicolor | 55,876 | 20-Aug-98 |
| | | GB_BA1:MTCY77 | 22255 | Z95389 | Mycobacterium tuberculosis H37Rv complete genome; segment 146/162. | Mycobacterium tuberculosis | 38,382 | 18-Jun-98 |
| rxa02041 | 737 | GB_BA1:MTCY210 | 36804 | Z84395 | Mycobacterium tuberculosis H37Rv complete genome; segment 34/162. | Mycobacterium tuberculosis | 70,041 | 17-Jun-98 |
| | | GB_BA1:MBS10OPER | 5962 | Y13228 | Mycobacterium bovis BCG DNA for ribosomal S10 operon. | Mycobacterium bovis BCG | 70,041 | 04-DEC-1997 |
| | | GB_BA1:MSGY42 | 36526 | AD000005 | Mycobacterium tuberculosis sequence from clone y42. | Mycobacterium tuberculosis | 39,945 | 03-DEC-1996 |
| rxa02042 | 537 | GB_BA1:MSGY42 | 36526 | AD000005 | Mycobacterium tuberculosis sequence from clone y42. | Mycobacterium tuberculosis | 42,015 | 03-DEC-1996 |
| | | GB_BA1:MTCY210 | 36804 | Z84395 | Mycobacterium tuberculosis H37Rv complete genome; segment 34/162. | Mycobacterium tuberculosis | 66,541 | 17-Jun-98 |
| | | GB_BA1:MLCB2492 | 37144 | Z98756 | Mycobacterium leprae cosmid B2492. | Mycobacterium leprae | 66,604 | 28-Aug-97 |
| rxa02043 | 241 | GB_BA1:AB017508 | 32050 | AB017508 | Bacillus halodurans C-125 genomic DNA, 32 kb fragment, complete cds. | Bacillus halodurans | 48,305 | 14-Apr-99 |
| | | GB_EST1:D41045 | 356 | D41045 | RICS3304A Rice shoot Oryza sativa cDNA, mRNA sequence. | Oryza sativa | 43,697 | 11-Nov-94 |
| | | GB_BA1:CZA382 | 42369 | AL078635 | Amycolatopsis orientalis cosmid pCZA382. | Amycolatopsis orientalis | 42,152 | 17-Aug-99 |
| rxa02077 | 519 | GB_RO:RNPLECT | 15231 | X59601 | Rat mRNA for plectin. | Rattus norvegicus | 41,468 | 19-DEC-1996 |
| | | GB_BA2:AF148219 | 1989 | AF148219 | Nostoc PCC8009 fibrillin and photosystem I protein E (psaE) genes, complete | Nostoc PCC8009 | 36,346 | 9-Jun-99 |
| | | | | | cds; and formamidopyrimidine-DNA glycosylase MutM (mutM) gene, partial | | | |
| | | | | | cds. | | | |
| | | GB_RO:RNPLECT | 15231 | X59601 | Rat mRNA for plectin. | Rattus norvegicus | 36,505 | 19-DEC-1996 |
| rxa02145 | 1740 | GB_BA1:MTCY190 | 34150 | Z70283 | Mycobacterium tuberculosis H37Rv complete genome; segment 98/162. | Mycobacterium tuberculosis | 64,033 | 17-Jun-98 |
| | | GB_BA1:MSGB1554CS | 36548 | L78814 | Mycobacterium leprae cosmid B1554 DNA sequence. | Mycobacterium leprae | 37,609 | 15-Jun-96 |
| | | GB_BA1:MSGB1551CS | 36548 | L78813 | Mycobacterium leprae cosmid B1551 DNA sequence. | Mycobacterium leprae | 37,609 | 15-Jun-96 |
| rxa02179 | 951 | GB_EST5:X93280 | 344 | X93280 | SSVIAET9 S. scrofa ovary Sus scrofa cDNA clone VIAet9 5′, | Sus scrofa | 39,649 | 14-MAY-1997 |
| | | GB_GSS11:AQ324492 | 859 | AQ324492 | mRNA sequence. mgxb0018H12r CUGI Rice Blast BAC Library Magnaporthe | Magnaporthe grisea | 36,151 | 8-Jan-99 |
| | | | | | grisea genomic clone mgxb0018H12r, genomic survey sequence. | | | |
| | | GB_HTG3:AC011395 | 95036 | AC011395 | Homo sapiens chromosome 5 clone CIT978SKB_18712, | Homo sapiens | 35,263 | 06-OCT-1999 |
| | | | | | ***SEQUENCING IN PROGRESS***, 6 unordered pieces. | | | |
| rxa02190 | 1581 | GB_BA1:MTCY01B2 | 35938 | Z95554 | Mycobacterium tuberculosis H37Rv complete genome; segment 72/162. | Mycobacterium tuberculosis | 76,141 | 17-Jun-98 |
| | | GB_BA1:MLACEA | 37049 | Z46257 | M. leprae aceA gene for isocitrate lyase. | Mycobacterium leprae | 72,172 | 22-MAY-1996 |
| | | GB_BA1:SC7H2 | 42655 | AL109732 | Streptomyces coelicolor cosmid 7H2. | Streptomyces coelicolor A3(2) | 40,627 | 2-Aug-99 |
| rxa02241 | 584 | GB_HTG3:AC010579 | 157658 | AC010579 | Drosophila melanogaster chromosome 3 clone BACR09D08 (D1101) RPCI- | Drosophila melanogaster | 40,402 | 24-Sep-99 |
| | | | | | 98 09.D.8 map 96F-96F strain y; cn bw sp, ***SEQUENCING IN PROGRESS | | | |
| | | | | | ***, 121 unordered pieces. | | | |
| | | GB_HTG3:AC010579 | 157658 | AC010579 | Drosophila melanogaster chromosome 3 clone BACR09D08 (D1101) RPCI- | Drosophila melanogaster | 40,402 | 24-Sep-99 |
| | | | | | 98 09.D.8 map 96F-96F strain y; cn bw sp, ***SEQUENCING IN PROGRESS | | | |
| | | | | | ***, 121 unordered pieces. | | | |
| | | GB_HTG2:AC007946 | 97610 | AC007946 | Drosophila melanogaster chromosome 3 clone BACR03O03 (D769) RPCI-98 | Drosophila melanogaster | 40,248 | 2-Aug-99 |
| | | | | | 03.O.3 map 96E-96F strain y; cn bw sp, ***SEQUENCING IN PROGRESS | | | |
| | | | | | ***, 70 unordered pieces. | | | |
| rxa02293 | 2511 | GB_BA1:SC7H2 | 42655 | AL109732 | Streptomyces coelicolor cosmid 7H2. | Streptomyces coelicolor A3(2) | 40,557 | 2-Aug-99 |
| | | GB_BA2:U49051 | 2859 | U49051 | Sinorhizobium meliloti putative DEAH family helicase HelO gene, complete | Sinorhizobium meliloti | 48,002 | 7-Aug-97 |
| | | | | | cds. | | | |
| | | GB_BA2:U32787 | 12361 | U32787 | Haemophilus influenzae Rd section 102 of 163 of the complete genome. | Haemophilus influenzae Rd | 36,309 | 29-MAY-1998 |
| rxa02357 | 6423 | GB_EST1:T47370 | 307 | T47370 | yb13b07.r1 Stratagene placenta (#937225) Homo sapiens cDNA clone | Homo sapiens | 40,850 | 1-Feb-95 |
| | | | | | IMAGE: 71029 5′ similar to contains L1 repetitive element, mRNA sequence. | | | |
| | | GB_PR1:HSVMYCLC2 | 11997 | Z15030 | H. sapiens gene for ventricular myosin light chain 2. | Homo sapiens | 40,012 | 9-Feb-99 |
| | | GB_PR1:HUMVMLC | 11997 | L01652 | Human ventricular myosin light chain 2 gene, seven exons. | Homo sapiens | 40,012 | 3-Aug-93 |
| rxa02359 | 1992 | GB_PR3:HS523G1 | 109885 | AL034375 | Human DNA sequence from clone 523G1 on chromosome 6p22.3-24.1 | Homo sapiens | 37,589 | 23-Nov-99 |
| | | | | | Contains part of the mRNA for SCA1 (spinocerebellar ataxia 1 | | | |
| | | | | | (olivopontocerebellar ataxia 1, autosomal dominant, ataxin 1)) ESTs, | | | |
| | | | | | STSs and GSSs, complete sequence. | | | |
| | | GB_PR3:HS523G1 | 109885 | AL034375 | Human DNA sequence from clone 523G1 on chromosome 6p22.3-24.1 | Homo sapiens | 35,888 | 23-Nov-99 |
| | | | | | Contains part of the mRNA for SCA1 (spinocerebellar ataxia 1 | | | |
| | | | | | (olivopontocerebellar ataxia 1, autosomal dominant, ataxin 1)) ESTs, | | | |
| | | | | | STSs and GSSs, complete sequence. | | | |
| rxa02363 | 4923 | GB_RO:MMU9842 | 168 | AJ009842 | Mus musculus cathepsin E gene, exon 3, partial. | Mus musculus | 44,643 | 19-Aug-99 |
| | | GB_RO:MMPROCNE | 1819 | X97399 | M. musculus mRNA for procathepsin E. | Mus musculus | 38,793 | 3-Jun-97 |
| | | GB_OM:PIGPEP | 1398 | M20920 | Swine pepsinogen A mRNA, complete cds. | Sus scrofa | 37,066 | 27-Apr-93 |
| rxa02369 | 2409 | GB_BA1:MTCY428 | 26914 | Z81451 | Mycobacterium tuberculosis H37Rv complete genome; segment 107/162, | Mycobacterium tuberculosis | 38,568 | 17-Jun-98 |
| | | GB_HTG3:AC010371 | 86848 | AC010371 | Homo sapiens chromosome 5 clone CITB-H1_2049O13, ***SEQUENCING | Homo sapiens | 41,055 | 15-Sep-99 |
| | | | | | IN PROGRESS***, 29 unordered pieces. | | | |
| | | GB_HTG3:AC010371 | 86848 | AC010371 | Homo sapiens chromosome 5 clone CITB-H1_2049O13, ***SEQUENCING | Homo sapiens | 41,055 | 15-Sep-99 |
| | | | | | IN PROGRESS***, 29 unordered pieces. | | | |
| rxa02370 | 915 | GB_IN2:AC005930 | 41284 | AC005930 | Leishmania major chromosome 3 clone L712 strain Friedlin, complete | Leishmania major | 39,686 | 13-Nov-99 |
| | | | | | sequence. | | | |
| | | GB_GSS3:B43984 | 403 | B43984 | HS-1058-B1-F09-MF.abi CIT Human Genomic Sperm Library C Homo | Homo sapiens | 39,918 | 21-OCT-1997 |
| | | | | | sapiens genomic clone Plate = CT 780 Col = 17 Row = L, genomic survey | | | |
| | | | | | sequence. | | | |
| | | GB_GSS9:AQ108989 | 192 | AQ108989 | CIT-HSP-2360J15.TF CIT-HSP Homo sapiens genomic clone 2360J15, | Homo sapiens | 39,474 | 29-Aug-98 |
| | | | | | genomic survey sequence. | | | |
| rxa02371 | 415 | GB_BA1:MTCY428 | 26914 | Z81451 | Mycobacterium tuberculosis H37Rv complete genome; segment 107/162. | Mycobacterium tuberculosis | 35,351 | 17-Jun-98 |
| | | GB_BA2:AE000749 | 16033 | AE000749 | Aquifex aeolicus section 81 of 109 of the complete genome. | Aquifex aeolicus | 38,677 | 25-MAR-1998 |
| | | GB_IN1:DMC63B12 | 36401 | AL021106 | Drosophila melanogaster cosmid clone 63B12. | Drosophila melanogaster | 38,592 | 29-Apr-99 |
| rxa02389 | 384 | GB_PR4:AC004617 | 176552 | AC004617 | Homo sapiens chromosome Y, clone 264, M, 20, complete sequence. | Homo sapiens | 40,212 | 13-OCT-1999 |
| | | GB_HTG1:HS27O5 | 149932 | AL033529 | Homo sapiens chromosome 1 clone RP1-27O5, ***SEQUENCING IN | Homo sapiens | 40,682 | 23-Nov-99 |
| | | | | | PROGRESS***, in unordered pieces. | | | |
| | | GB_HTG1:HS27O5 | 149932 | AL033529 | Homo sapiens chromosome 1 clone RP1-27O5, ***SEQUENCING IN | Homo sapiens | 40,682 | 23-Nov-99 |
| | | | | | PROGRESS***, in unordered pieces. | | | |
| rxa02419 | 315 | GB_BA1:MTCY06H11 | 38000 | Z85982 | Mycobacterium tuberculosis H37Rv complete genome; segment 73/162. | Mycobacterium tuberculosis | 54,341 | 17-Jun-98 |
| | | GB_GSS12:AQ410661 | 433 | AQ410661 | HS_5045_B2_E05_SP6E RPCI-11 Human Male BAC Library Homo sapiens | Homo sapiens | 35,505 | 17-MAR-1999 |
| | | | | | genomic clone Plate = 621 Col = 10 Row = J, genomic survey sequence. | | | |
| | | GB_BA2:SCAHBAGC2 | 5367 | AF131879 | Streptomyces collinus ansatrienin AHBA biosynthetic gene cluster region 2, | Streptomyces collinus | 35,593 | 24-MAY-1999 |
| | | | | | complete sequence. | | | |
| rxa02420 | 504 | GB_BA1:MTCY06H11 | 38000 | Z85982 | Mycobacterium tuberculosis H37Rv complete genome; segment 73/162. | Mycobacterium tuberculosis | 64,542 | 17-Jun-98 |
| | | GB_BA1:SCI35 | 40909 | AL031541 | Streptomyces coelicolor cosmid I35. | Streptomyces coelicolor | 36,439 | 9-Sep-98 |
| | | GB_EST37:AI995529 | 634 | AI995529 | 701675731 A. thaliana, Columbia Col-0, inflorescence-1 Arabidopsis thaliana | Arabidopsis thaliana | 38,200 | 8-Sep-99 |
| | | | | | cDNA clone 701675731, mRNA sequence. | | | |
| rxa02468 | 1347 | GB_BA1:MTCY7D11 | 22070 | Z95120 | Mycobacterium tuberculosis H37Rv complete genome; segment 138/162. | Mycobacterium tuberculosis | 60,654 | 17-Jun-98 |
| | | GB_HTG4:AC008754 | 86446 | AC008754 | Homo sapiens chromosome 19 clone CITB-E1_3023J11, ***SEQUENCING | Homo sapiens | 35,634 | 31-OCT-1999 |
| | | | | | IN PROGRESS***, 73 unordered pieces. | | | |
| | | GB_HTG4:AC008754 | 86446 | AC008754 | Homo sapiens chromosome 19 clone CITB-E1_3023J11, ***SEQUENCING | Homo sapiens | 35,634 | 31-OCT-1999 |
| | | | | | IN PROGRESS***, 73 unordered pieces. | | | |
| rxa02522 | 666 | GB_PL2:AC004135 | 73805 | AC004135 | Genomic sequence for Arabidopsis thaliana BAC T17H7 from Chromosome 1, | Arabidopsis thaliana | 38,052 | 29-MAY-1999 |
| | | | | | complete sequence. | | | |
| | | GB_PL2:AC004135 | 73805 | AC004135 | Genomic sequence for Arabidopsis thaliana BAC T17H7 from Chromosome 1, | Arabidopsis thaliana | 36,157 | 29-MAY-1999 |
| | | | | | complete sequence. | | | |
| | | GB_RO:MUSTRAA | 6149 | M36386 | Mouse tumor rejection antigen P815A gene, complete cds. | Mus musculus | 35,769 | 27-Apr-93 |
| rxa02533 | | | | | | | | |
| rxa02615 | 759 | GB_BA1:MSKATG | 2307 | X98718 | M. smegmatis katG gene. | Mycobacterium smegmatis | 37,349 | 16-Jan-97 |
| | | GB_BA2:MSU46844 | 16951 | U46844 | Mycobacterium smegmatis catalase-peroxidase (katG), putative arabinosyl | Mycobacterium smegmatis | 39,783 | 12-MAY-1997 |
| | | | | | transferase (embC, embA, embB), genes complete cds and putative propionyl- | | | |
| | | | | | coA carboxylase beta chain (pccB) genes, partial cds. | | | |
| | | GB_BA2:AF124600 | 4115 | AF124600 | Corynebacterium glutamicum chorismate synthase (aroC), shikimate kinase | Corynebacterium glutamicum | 39,893 | 04-MAY-1999 |
| | | | | | (aroK), and 3-dehydroquinate synthase (aroB) genes, complete cds; and | | | |
| | | | | | putative cytoplasmic peptidase (pepQ) gene, partial cds. | | | |
| rxa02633 | 387 | GB_RO:RNY09164 | 6556 | Y09164 | R. norvegicus mRNA for sodium channel. | Rattus norvegicus | 34,204 | 8-Jan-97 |
| | | GB_RO:RNY09164 | 6556 | Y09164 | R. norvegicus mRNA for sodium channel. | Rattus norvegicus | 38,298 | 8-Jan-97 |
| rxa02635 | 357 | GB_BA1:MTV018 | 53450 | AL021899 | Mycobacterium tuberculosis H37Rv complete genome; segment 90/162. | Mycobacterium tuberculosis | 37,464 | 18-Jun-98 |
| | | GB_GSS1:MTAF001381 | 3045 | AF001381 | Mycobacterium tuberculosis strain KIT10218 cosmid 10R, partial sequence, | Mycobacterium tuberculosis | 36,667 | 9-Aug-97 |
| | | | | | genomic survey sequence. | | | |
| | | GB_BA1:SC6C5 | 18160 | AL034492 | Streptomyces coelicolor cosmid 6C5. | Streptomyces coelicolor | 42,938 | 14-DEC-1998 |
| rxa02636 | 285 | GB_BA1:MTV018 | 53450 | AL021899 | Mycobacterium tuberculosis H37Rv complete genome; segment 90/162. | Mycobacterium tuberculosis | 41,036 | 18-Jun-98 |
| | | GB_GSS1:MTAF001381 | 3045 | AF001381 | Mycobacterium tuberculosis strain KIT10218 cosmid 10R, partial sequence, | Mycobacterium tuberculosis | 40,206 | 9-Aug-97 |
| | | | | | genomic survey sequence. | | | |
| | | GB_GSS1:MTAF001381 | 3045 | AF001381 | Mycobacterium tuberculosis strain KIT10218 cosmid 10R, partial sequence, | Mycobacterium tuberculosis | 45,091 | 9-Aug-97 |
| | | | | | genomic survey sequence. | | | |
| rxa02637 | 426 | GB_PR4:AC006602 | 93610 | AC006602 | Homo sapiens Chromosome 15q11-q13 PAC clone pDJ476i9 from the Prader- | Homo sapiens | 37,441 | 23-Feb-99 |
| | | | | | Willi/Angelman Syndrome region, complete sequence. | | | |
| | | GB_PR4:AC007275 | 169904 | AC007275 | Homo sapiens clone NH0109F19, complete sequence. | Homo sapiens | 36,170 | 29-Jul-99 |
| | | GB_PR4:AC007275 | 169904 | AC007275 | Homo sapiens clone NH0109F19, complete sequence. | Homo sapiens | 34,783 | 29-Jul-99 |
| rxa02657 | 3705 | GB_BA1:MTCY48 | 35377 | Z74020 | Mycobacterium tuberculosis H37Rv complete genome; segment 69/162. | Mycobacterium tuberculosis | 63,678 | 17-Jun-98 |
| | | GB_BA1:MLCL458 | 43839 | AL049478 | Mycobacterium leprae cosmid L458. | Mycobacterium leprae | 63,716 | 27-Aug-99 |
| | | GB_BA1:MSGB13GS | 42923 | L78823 | Mycobacterium leprae cosmid B13 DNA sequence. | Mycobacterium leprae | 37,939 | 15-Jun-96 |
| rxa02682 | 450 | GB_PR2:AC002379 | 118595 | AC002379 | Human BAC clone GS165I04 from 7q21, complete sequence. | Homo sapiens | 36,552 | 23-Jul-97 |
| | | GB_EST18:T44994 | 452 | T44994 | 8257 Lambda-PRL2 Arabidopsis thaliana cDNA clone 127P23T7, mRNA | Arabidopsis thaliana | 34,247 | 7-Jan-98 |
| | | | | | sequence. | | | |
| | | GB_EST14:M395030 | 444 | AA395030 | 26827 Lambda-PRL2 Arabidopsis thaliana cDNA clone 111K20XP 3′, mRNA | Arabidopsis thaliana | 31,806 | 30-OCT-1997 |
| | | | | | sequence. | | | |
| rxa02752 | 618 | GB_BA1:MTCY274 | 39991 | Z74024 | Mycobacterium tuberculosis H37Rv complete genome; segment 126/162. | Mycobacterium tuberculosis | 38,715 | 19-Jun-98 |
| | | GB_BA1:SC2E1 | 38962 | AL023797 | Streptomyces coelicolor cosmid 2E1. | Streptomyces coelicolor | 53,300 | 4-Jun-98 |
| | | GB_BA2:SKZ86111 | 7860 | Z86111 | Streptomyces lividans rpsP, trmD, rplS, sipW, sipX, sipY, sipZ, mutT genes | Streptomyces lividans | 52,970 | 27-OCT-1999 |
| | | | | | and 4 open reading frames. | | | |
| rxa02755 | 2118 | GB_BA1:MSGY151 | 37036 | AD000018 | Mycobacterium tuberculosis sequence from clone y151. | Mycobacterium tuberculosis | 56,905 | 10-DEC-1996 |
| | | GB_BA1:MTCY130 | 32514 | Z73902 | Mycobacterium tuberculosis H37Rv complete genome; segment 59/162. | Mycobacterium tuberculosis | 39,419 | 17-Jun-98 |
| | | GB_BA1:SC4H2 | 38400 | AL022268 | Streptomyces coelicolor cosmid 4H2. | Streptomyces coelicolor | 56,729 | 6-Apr-98 |
| rxa02764 | | | | | | | | |
| rxa02819 | 1070 | GB_GSS14:AQ549674 | 479 | AQ549674 | RPCI-11-413C1.TV RPCI-11 Homo sapiens genomic clone RPCI-11-413C1, | Homo sapiens | 40,705 | 28-MAY-1999 |
| | | | | | genomic survey sequence. | | | |
| | | GB_GSS14:AQ549674 | 479 | AQ549674 | RPCI-11-413C1.TV RPCI-11 Homo sapiens genomic clone RPCI-11-413C1, | Homo sapiens | 38,696 | 28-MAY-1999 |
| | | | | | genomic survey sequence. | | | |
| rxa02826 | 558 | GB_BA1:MTY20H10 | 35980 | Z92772 | Mycobacterium tuberculosis H37Rv complete genome; segment 31/162. | Mycobacterium tuberculosis | 67,387 | 17-Jun-98 |
| | | GB_BA1:SGNUSG | 7235 | X72787 | S. griseus nusG, rpIKAJL gene cluster. | Streptomyces griseus | 65,946 | 06-MAY-1998 |
| | | GB_BA1:STMVBRA1 | 7409 | D50624 | Streptomyces virginiae VbrA gene for NusG like protein, SecE like protein and | Streptomyces virginiae | 65,766 | 10-Feb-99 |
| | | | | | ribosomal protein, aspartate aminotransferase and adenosine deaminase, | | | |
| | | | | | complete cds. | | | |
| rxa02833 | 906 | EM_PAT:E11161 | 3521 | E11161 | Genomic DNA including an autonomous replication sequences (ars) | Corynebacterium glutamicum | 98,343 | 08-OCT-1997 |
| | | | | | | | | (Rel. 52, |
| | | | | | | | | Created) |
| | | GB_BA2:MAU19185 | 3952 | U19185 | Mycobacterium avium RpmH (rpmH) and DnaA (dnaA) genes, complete cds; | Mycobacterium avium | 46,333 | 08-DEC-1998 |
| | | | | | and DnaN (dnaN) gene, partial cds. | | | |
| | | GB_BA1:MLUDNAA | 4171 | M34006 | M. luteus ribonuclease P (mpA), 50S ribosomal subunit protein L34 (rpmH), | Micrococcus luteus | 46,540 | 16-Feb-94 |
| | | | | | DNA biosynthesis initiation protein (dnaA), and DNA polymerase III beta | | | |
| | | | | | subunit (dnaN) genes, complete cds. | | | |

What is claimed:

1. An isolated nucleic acid molecule selected from the group consisting of
a) an isolated nucleic acid molecule comprising the nucleotide sequence set forth in SEQ ID NO:383, or a complement thereof;
b) an isolated nucleic acid molecule which encodes a polypeptide comprising the amino acid sequence set forth in SEQ ID NO:384, or a complement thereof;
c) an isolated nucleic acid molecule which encodes a naturally occurring allelic variant of a polypeptide comprising the amino acid sequence set forth in SEQ ID NO:384, or a complement thereof,
d) an isolated nucleic acid molecule comprising a nucleotide sequence which is at least 50% identical to the entire nucleotide sequence set forth in SEQ ID NO:383, or a complement thereof; and
e) an isolated nucleic acid molecule comprising a fragment of at least 15 contiguous nucleotides of the nucleotide sequence set forth in SEQ ID NO:383, or a complement thereof.

2. An isolated nucleic acid molecule comprising the nucleic acid molecule of claim 1 and a nucleotide sequence encoding a heterologous polypeptide.

3. A vector comprising the nucleic acid molecule of claim 1.

4. The vector of claim 3, which is an expression vector.

5. A host cell transfected with the expression vector of claim 4.

6. The host cell of claim 5, wherein said cell is a microorganism.

7. The host cell of claim 6, wherein said cell belongs to the genus Corynebacterium or Brevibacterium.

8. A method of producing a polypeptide comprising culturing the host cell of claim 5 in an appropriate culture medium to, thereby, produce the polypeptide.

9. A method for producing a fine chemical, comprising culturing the cell of claim 5 such that the fine chemical is produced.

10. The method of claim 9, wherein said method further comprises the step of recovering the fine chemical from said culture.

11. The method of claim 9, wherein said cell belongs to the genus Corynebacterium or Brevibacterium.

12. The method of claim 9, wherein said cell is selected from the group consisting of Corynebacterium glutamicum, Corynebacterium herculis, Corynebacterium, lilium, Corynebacterium acetoacidophilum, Corynebacterium acetoglutamicum, Corynebacterium acetophilum, Corynebacterium ammoniagenes, Corynebacterium fujiokense, Corynebacterium nitrilophilus, Brevibacterium ammoniagenes, Brevibacterium butanicum, Brevibacterium divaricatum, Brevibacterium flavum, Brevibacterium healii, Brevibacterium ketoglutamicum, Brevibacterium ketosoreductum, Brevibacterium lactofermentum, Brevibacterium linens, Brevibacterium paraffinolyticum, and those strains set forth in Table 3.

13. The method of claim 9, wherein expression of the nucleic acid molecule from said vector results in modulation of production of said fine chemical.

14. The method of claim 9, wherein said fine chemical is selected from the group consisting of organic acids, proteinogenic and nonproteinogenic amino acids, purine and pyrimidine bases, nucleosides, nucleotides, lipids, saturated and unsaturated fatty acids, diols, carbohydrates, aromatic compounds, vitamins, cofactors, polyketides, and enzymes.

15. The method of claim 9, wherein said fine chemical is an amino acid selected from the group consisting of lysine, glutamate, glutamine, alanine, aspartate, glycine, serine, threonine, methionine, cysteine, valine, leucine, isoleucine, arginine, proline, histidine, tyrosine, phenylalanine, and tryptophan.

16. An isolated polypeptide selected from the group consisting of
a) an isolated polypeptide comprising the amino acid sequence set forth in SEQ ID NO:384;
b) an isolated polypeptide comprising a naturally occurring allelic variant of a polypeptide comprising the amino acid sequence set forth in SEQ ID NO:384;
c) an isolated polypeptide which is encoded by the nucleotide sequence set forth in SEQ ID NO:383;
d) an isolated polypeptide which is encoded by a nucleic acid molecule comprising a nucleotide sequence which is at least 50% identical to the entire nucleotide sequence set forth in SEQ ID NO:383;
e) an isolated polypeptide comprising an amino acid sequence which is at least 50% identical to the entire amino acid sequence set forth in SEQ ID NO:384; and
f) an isolated polypeptide comprising a fragment of a polypeptide comprising the amino acid sequence set forth in SEQ ID NO:384, wherein said polypeptide fragment maintains a biological activity of the polypeptide comprising the amino sequence.

17. The isolated polypeptide of claim 16, wherein said polypeptide is involved in the production of a fine chemical.

18. The isolated polypeptide of claim 16, further comprising heterologous amino acid sequences.

19. A method for diagnosing the presence or activity of Corynebacterium diphtheriae in a subject, comprising detecting the presence of at least one of the nucleic acid molecules of claim 1, thereby diagnosing the presence or activity of Corynebacterium diphtheriae in the subject.

20. A method for diagnosing the presence or activity of Corynebacterium diphtheriae in a subject, comprising detecting the presence of at least one of the polypeptide molecules of claim 16, thereby diagnosing the presence or activity of Corynebacterium diphtheriae in the subject.

21. A host cell comprising a nucleic acid molecule selected from the group consisting of
a) a nucleic acid molecule comprising the nucleotide sequence set forth in SEQ ID NO:383, wherein the nucleic acid molecule is disrupted by at least one technique selected from the group consisting of a point mutation, a truncation, an inversion, a deletion, an addition, a substitution and homologous recombination;
b) a nucleic acid molecule comprising the nucleotide sequence set forth in SEQ ID NO:383, wherein the nucleic acid molecule comprises one or more nucleic acid modifications as compared to the sequence set forth in SEQ ID NO:383, wherein the modification is selected from the group consisting of a point mutation, a truncation, an inversion, a deletion, an addition and a substitution; and
c) a nucleic acid molecule comprising the nucleotide sequence set forth in SEQ ID NO:383, wherein the regulatory region of the nucleic acid molecule is modified relative to the wild-type regulatory region of the molecule by at least one technique selected from the group consisting of a point mutation, a truncation, an inversion, a deletion, an addition, a substitution and homologous recombination.
